(19) United States
(12) Patent Application Publication
(10) Pub. No.: US 2026/0325639 A1
(43) Pub. Date: Oct. 22, 2026

(54) CHIMERIC ANTIGEN RECEPTOR COMPOSITIONS, CAR-MAST CELLS FORMED THEREFROM, AND METHODS OF USE THEREOF

(71) Applicant: YALE UNIVERSITY, New Haven, CT (US)

(72) Inventors: Xiaolei SU, New Haven, CT (US); Jianjian GUO, New Haven, CT (US); Yiwei XIONG, New Haven, CT (US)

(21) Appl. No.: 19/484,878

(22) PCT Filed: May 15, 2024
(86) PCT No.: PCT/US2024/029466

§ 371 Date: Nov. 17, 2025

Related U.S. Application Data
(60) Provisional application No. 63/502,349, filed on May 15, 2023.

(51) Int. Cl. A61K 40/42; A61K 40/31; A61P 35/00; C07K 14/705; C07K 16/28; C07K 16/30; C07K 16/32

(52) U.S. Cl. CPC A61K 40/4211; A61K 40/31; A61K 40/4205; A61K 40/4258; A61P 35/00; C07K 14/705; C07K 14/70503; C07K 14/7051; C07K 14/70535; C07K 16/2803; C07K 16/3084; C07K 16/32; C07K 2319/02; C07K 2319/03

(57) ABSTRACT

Fusion proteins including (a) a mast cell intracellular signaling domain; and (b) an amino acid sequence that is heterologous to the mast cell intracellular signaling domain are provided. In some embodiments, (a) includes the intracellular domain of IgE receptor (FcεRI), a cytokine receptor (c-KIT), a G-protein-coupled receptor, or a toll-like receptor expressed in mast cells, or functional fragment or variant thereof. In some embodiments, the amino acid sequence of (a) includes an ITAM sequence. In preferred embodiments, the fusion proteins are chimeric antigen receptors (referred to as “CAR-mast”), that include a transmembrane domain and an extracellular domain that targets an antigen. Nucleic acids encoding the fusion proteins, and mast cells including or expressing the nucleic acids and/or fusion proteins are also provided, as are methods of the using the same to treat diseases and disorders characterized by expression of the antigen.

### CROSS-REFERENCE TO RELATED APPLICATIONS

This application claims the benefit of and priority to U.S. Ser. No. 63/502,349, filed May 15, 2023, which is incorporated by reference herein in its entirety.

### STATEMENT REGARDING FEDERALLY SPONSORED RESEARCH

This invention was made with government support under GM138299 and CA121974 awarded by the National Institutes of Health. The Government has certain rights in the invention.

### REFERENCE TO THE SEQUENCE LISTING

The Sequence Listing XML submitted as a file named “YU_8680_PCT_ST26.xml” and having a size of 38,099 bytes is hereby incorporated by reference pursuant to 37 C.F.R. § 1.834(c)(1).

### FIELD OF THE INVENTION

The disclosed invention is generally in the field of immunology and specifically in the area of engineered therapeutic mast cells.

### BACKGROUND OF THE INVENTION

CAR (Chimeric antigen receptor)-T cell therapy represents a major advancement in treating refractory or relapsed blood cancers with remarkable efficacy; however, CARs targeting solid tumors showed limited effects¹⁻³. Major challenges include T cells' poor infiltration into the tumor tissue, exhaustion and low persistence under an immune-suppressive tumor microenvironment (TME)⁴⁻⁶. To tackle these problems, a plethora of studies focused on modifying the CAR design and genetically manipulating T cell genome to improve CAR-T's function. These approaches led to promising outcomes. Meanwhile, T cells compose one of the many players in the anti-tumor immune response. The modular property of CAR, of which multiple signaling domains (even from receptors out of T cells) can be incorporated to trigger a variety of different signaling responses^{7,8}, indicate CAR as a versatile tool to reprogram multiple cell types. Seeking alternative immune cells for CAR carriers could fundamentally solve some of the major challenges in CAR-T. Indeed, two other CAR effector cells, CAR-NK cells and CAR-macrophages have been recently developed and showed encouraging effects in reducing tumor burden⁹⁻¹¹. However, they are still at the early stage and limitations exist. For example, NK cells are short lived which might compromise the long-term anti-tumor effect whereas macrophages can sometimes promote tumor growth and suppress T cell responses¹². And the clinical outcomes of these new types of CAR cells remained to be fully evaluated. Therefore, a need remained for developing alternative CAR carriers that could provide new strategies to address the above issues.

Thus, it is an object of the invention to provide compositions and methods for improved therapeutic CAR cells.

### BRIEF SUMMARY OF THE INVENTION

It has been discovered that chimeric antigen receptors (CAR) can be constructed using a mast cell intracellular signaling domain in addition or alternative to a traditional CAR intracellular domain. As exemplified in the disclosed experiments, such “CAR-mast” constructs when introduced into mast cells can specifically kill cancer cells in vitro and release chemokines that recruit T cells and NK cells, e.g., to the tumor tissues and inhibit solid tumor growth and in mouse xenograft models.

Thus provided are fusion polypeptides (also referred to as fusion proteins) including (a) a mast cell intracellular signaling domain; and (b) an amino acid sequence that is heterologous to the mast cell intracellular signaling domain are provided. In some embodiments, (a) includes the intracellular domain of IgE receptor (FcεRI), a cytokine receptor (c-KIT), a G-protein-coupled receptor, or a toll-like receptor expressed in mast cells, or functional fragment or variant thereof. In some embodiments, the amino acid sequence of (a) includes an ITAM sequence such as DGVYTGLSTRNQETYETLKHE (SEQ ID NO:11) or a functional variant thereof, or variant thereof having an amino acid sequence at least 70%, 75%, 80%, 85%, 90%, 95%, or 99% identical to SEQ ID NO: 1l. In some embodiments, the amino acid sequence of (a) includes the amino acid sequence of any one of SEQ ID NOS:11-21, or a functional fragment thereof, or a variant thereof having an amino acid sequence at least 70%, 75%, 80%, 85%, 90%, 95%, or 99% identical to any one of SEQ ID NOS:11-21.

In some embodiments, the (b) includes one or more of an extracellular domain, transmembrane domain, and/or a further intracellular domain. In some embodiments, the fusion polypeptide is a chimeric antigen receptor (CAR) (also referred to herein as a CAR-mast). Thus, in particular embodiments, the CAR includes an intracellular cellular domain comprising (a), and (b) includes a transmembrane domain, and an extracellular domain. In some embodiments, the transmembrane domain and optionally stalk of CD8α, optionally of SEQ ID NO:4 or fragment or variant thereof with at least 70% sequence identity thereto, or CD28, optionally SEQ ID NO:10 or fragment or variant thereof with at least 70% sequence identity thereto.

In some embodiments, the CAR is specific for an antigen selected from the group consisting of a cancer antigen, an inflammatory disease antigen, a neuronal disorder antigen, HIV/AIDS, a diabetes antigen, a cardiovascular disease antigen, an infectious disease antigen (including a viral antigen, a protozoan antigen, a bacterial antigen, and an allergen), an autoimmune disease antigen and an autoimmune disease antigen, or combinations thereof. Exemplary target antigens include, for example, CD19, GD2, B7H3, AFP, AKAP 4, ALK, Androgen receptor, B7H3, BCMA, Bcr Abl, BORIS, Carbonic, CD123, CD138, CD174, CD20, CD22, CD30, CD33, CD38, CD80, CD86, CEA, CEACAM5, CEACAM6, Cyclin, CYP1B1, EBV, EGFR, EGFR806, EGFRvIII, EpCAM, EphA2, ERG, ETV6 AML, FAP, Fos related antigen1, Fucosyl, fusion, GD3, GloboH, GM3, gp100, GPC3, HER 2/neu, HER2, HMWMAA, HPV E6/E7, hTERT, Idiotype, IL12, IL13RA2, IM19, IX, LCK, Legumain, IgK, LMP2, MAD CT 1, MAD CT 2, MAGE, MelanA/MART1, Mesothelin, MET, ML IAP, MUC1, Mutant p53, MYCN, NA17, NKG2D L, NY BR 1, NY ESO 1, NY ESO 1, OY TES1, p53, Page4, PAP, PAX3, PAX5, PD L1, PDGFR β, PLAC1, Polysialic acid, Proteinase3 (PR1), PSA, PSCA, PSMA, Ras mutant, RGS5, RhoC, ROR1, SART3, sLe(a), Sperm protein 17, SSX2, STn, Survivin, Tie2, Tn, TRP 2, Tyrosinase, VEGFR2, WT1, XAGE, Claudin-6, Claudin-18.2 and CD70.

In some embodiments, the target antigen is a cancer antigen, such as CD19, GD2, B7H3, 41BB, 5T4, adenocarcinoma antigen, alpha fetoprotein, BAFF, B lymphoma cell, C242 antigen, CA 125, carbonic anhydrase 9 (CA IX), C MET, CCR4, CD152, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNT0888, CTLA 4, DR5, EGFR, EpCAM, CD3, FAP, fibronectin extra domain B, folate receptor 1, GD3 ganglioside, glycoprotein 75, GPNMB, HER2/neu, HER, HGF, human scatter factor receptor kinase, IGF 1 receptor, IGF I, IgG1, L1 CAM, IL 13, IL 6, insulin-like growth factor I receptor, integrin α5β1, integrin αvβ3, MORAb 009, MS4A1, MUC1, mucin CanAg, N glycolylneuraminic acid, NPC 1C, PDGF R a, PDL192, phosphatidylserine, prostatic carcinoma cells, RANKL, RON, ROR1, SCH 900105, SDC1, SLAMF7, TAG 72, tenascin C, TGF beta 2, TGF β, TRAIL R1, TRAIL R2, tumor antigen CTAA16.88, VEGF A, VEGFR 1, VEGFR2, or vimentin.

In some embodiments, the CAR includes an extracellular domain having an anti-CD19, anti-GD2, anti-B7H3, or anti-HER2 antigen binding domain, optionally of SEQ ID NOS:2, 8, 9, and SEQ ID NO:34, respectfully, or a fragment or variant thereof comprising the complementary determining regions (CDRs) thereof.

Nucleic acids encoding the fusion polypeptides, such as RNA, including, but not limited to mRNA, and DNA nucleic acids, are also provided. In some embodiments, the nucleic acid includes an expression control sequence(s) such as promoter. In some embodiments, the nucleic acid is a vector or a transposon. Exemplary vectors including expression vectors such plasmids, cosmids, and replicons, and viral vectors such as a lentiviral vector, an Adeno-associated virus (AAV) vector, an adenovirus vector, a Herpes Simplex virus (HSV) vector, a vesicular stomatitis (VSV) vector, or a human Bocavirus vector (hBoV), or a chimeric vector including a combination of any two or more of an Adeno-associated virus (AAV) vector, Herpes Simplex virus (HSV) vector, vesicular stomatitis (VSV) vector, or a human Bocavirus vector (hBoV).

Isolated cells including or expression the polypeptides and/or nucleic acids are also provided. In some embodiments, the cells are mast cells, mast progenitor cells, or hematopoietic stem cells (HSC). The cells can be isolated from a subject in need of adoptive cell therapy. Pharmaceutical compositions comprising a population of isolated cells and a pharmaceutically acceptable buffer, carrier, diluent, or excipient are also provided, as are methods of use thereof.

An exemplary method of treating a subject having a disease, disorder, or condition can include administering to the subject an effective amount of the pharmaceutical composition including isolated cells. In preferred methods, the subject has a disease, disorder, or condition associated with an elevated expression or specific expression of an antigen, and the method includes administering to the subject an effective amount of cells including a CAR-mast targeting the antigen. In some embodiments, the cells are isolated or derived from the subject having the disease, disorder, or condition prior to the introduction to the cell or from a healthy donor. In some embodiments, the cells are mast cells isolated as mast progenitor cells or hematopoietic stem cells and differentiated ex vivo into mast cells. In preferred embodiments, the subject is a human. In some embodiments, the subject has cancer, optionally a cancer one or more solid tumors associated therewith.

Additional advantages of the disclosed method and compositions will be set forth in part in the description which follows, and in part will be understood from the description, or can be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings illustrate several embodiments of the disclosed method and compositions and together with the description, serve to explain the principles of the disclosed method and compositions.

FIGS. 1A-1C illustrate the generation of exemplary CAR-mast cells. FIG. 1A is a schematic of the generation of mouse CAR-mast cells. FIG. 1B is a micrograph image of mouse mast cells stained with toluidine blue 49 days after in vitro culture. Granules were indicated by purple condensates around the nucleus. FIG. 1C is a density plot showing surface staining of c-kit and FcεR1α of Bulk bone marrow cells and spleen cells cultured at day 0 (left) and day 59 (right).

FIGS. 2A-2E illustrate exemplary CD19 CAR-mast cells. FIG. 2A is a schematic of an exemplary CD19 CAR construct. FIG. 2B is a graph showing CD19 CAR expression on mouse mast cells. FIG. 2C is a bar graph showing TNFα release determined by ELISA. Mouse CAR mast cells (CAR+) or wildtype mast cells (CAR−) were cocultured with hCD19+B16 or plain B16 cells for 1 day at 37° C., at E:T=1:1. FIG. 2D is a bar graph showing cytotoxicity of CAR-mast cells determined by luciferase assay. Mouse CAR mast cells (CAR+) or wildtype mast cells (CAR−) were cocultured with hCD19+B16 or plain B16 cells for 2 days at 37° C., at E:T=4:1. FIG. 2E is a heat map showing chemokine and cytokine profiling of CAR-mast cells. Mouse CAR mast cells (CAR+) or wildtype mast cells (CAR−) were cocultured with hCD19+B16 or plain B16 cells for 1 day at 37° C., at E:T=3:1.

FIG. 3A-3F show exemplary GD2 CAR-mast cells. FIG. 3A is a schematic of an exemplary GD2 CAR construct. FIG. 3B is a graph showing the GD2 CAR expression on mouse mast cells. FIG. 3C are bar graphs showing surface expression of CD107a release (left) and TNFα release (right). Mouse CAR mast cells (CAR+) or wildtype mast cells (CAR−) were cocultured with GD2+MC38 or plain MC38 cells for 1 day at 37° C., at E:T=2:1. FIG. 3D is a line graph showing the cytotoxicity of CAR-mast cells determined by luciferase assay. Mouse CAR mast cells (CAR+) or wildtype mast cells (CAR−) were cocultured with GD2+MC38 or plain MC38 cells for 2 Days at 37° C. FIG. 3E is a heat map of chemokine and cytokine profiling of CAR-mast cells. Mouse CAR mast cells (CAR+) or wildtype mast cells (CAR−) were cocultured with GD2+MC38 or plain M38 cells for 1 day at 37° C., at E:T=2:1. FIG. 3F shows killing by MC38-GD2-mCherry-Luciferase cells (GD2+) after 24 hr coculture at 37° C. at the E:T of 0.008:1, 0.04:1, 0.2:1, 1:1, 5:1 respectively.

FIGS. 4A-4F show exemplary B7H3 CAR-mast cells. FIG. 4A is a schematic of an exemplary B7H3 CAR construct. FIG. 4B is a graph showing B7H3 CAR expression on mouse mast cells. FIG. 4C are bar graphs showing surface expression of CD107a release (left) and TNFα release (right). Mouse CAR mast cells (CAR+) or wildtype mast cells (CAR−) were cocultured with GD2+MC38 or plain MC38 cells for 1 day at 37° C., at E:T=3:1. FIG. 4D is a line graph showing cytotoxicity of CAR-mast cells determined by luciferase assay. Mouse CAR mast cells (CAR+) or wildtype mast cells (CAR−) were cocultured with GD2+MC38 or plain MC38 cells for 2 days at 37° C. FIG. 4E is a heat map showing chemokine and cytokine profiling of CAR-mast cells. Mouse CAR mast cells (CAR+) or wildtype mast cells (CAR−) were cocultured with GD2+MC38 or plain M38 cells for 1 day at 37° C., at E:T=4:1. FIG. 4F is a graph showing CAR mast cells killing of a mixture of MC38-B7H3 cells (Antigen+) and MC38-mCherry-Luciferase cells (Antigen-) after 21 hr coculture at 37° C. at the E:T of 1:1, 3:1, 6:1 respectively.

FIGS. 5A-5C show the anti-tumor effect of exemplary B7H3 CAR-mast cells. FIG. 5A is a schematic of an experimental timeline of CAR-mast cell treatment in a mouse xenograft model. FIG. 5B is a line graph of tumor growth in individual mouse receiving 2 doses of CAR-mast cells, wildtype mast cells or PBS. FIG. 5C is a survival curve of mice monitored in FIG. 5B. Endpoint was determined by death of the animal or exceeding of tumor size over 2000 mm³.

FIG. 6A is a heat map showing the results of cytokine profiling of B7-H3 CAR mast cells (B7H3 CAR) or GD2 CAR mast cells (GD2 CAR) activated by either coculturing with MC38-B7H3-mCherry-Luciferase cells (B7H3-MC38) or MC38-GD2-mCherry-Luciferase cells (GD2-MC38) by coculture at E:T of 1:1 respectively, or by IgE, where the CAR mast cells are sensitized with 1 ug/mL anti-DNP IgE for 1 hr then cocultured with 20 ng/mL DNP for 20 hr at 37° C. FIG. 6B is a graph showing antigen-specific killing of MC38-B7H3-mCherry-Luciferase cells(B7H3+) treated with either B7H3 CAR mast cells (CAR), wildtype mast cells (WT) or IgE-activated wildtype mast cells (WT+IgE) at the E:T of 1:1, 3:1, 6:1 respectively with cancer cells total number at 0.02M. FIG. 6C is a diagram of mouse CAR-mast cell anaphylactic responses evaluation in mouse MC38-B7H3 tumor model. Timing of mast cell injections are indicated by the arrows. FIG. 6D is a graph showing rectal temperature at various timepoints after treatment with CAR-mast cells (CAR, N=5), wildtype mast cells (WT, N=5), PBS vehicle (PBS, N=5). FIG. 6E is a bar graph showing plasma histamine concentration of the treated mice.

FIG. 7A is a series of scatter plots showing HER2 expression on CAR-mast cells compared to Control CD19 CAR-mast cells. FIG. 7B is a scatter plot showing surface expressions of lineage markers (e.g., cKit, FceRa) on HER2 CAR-mast cells. FIG. 7C is a line graph showing the results of a Degranulation/Activation assay, as measured by CD107 staining for CAR-mast cells. FIG. 7D is a bar graph showing TNFα production in the supernatant of mouse CAR mast cells cocultured with HER2-EMT6 cells at the indicated effector-to-target (E:T) ratios after 48 hr. FIG. 7E is a heat map showing the results of cytokine and chemokine profiles of HER2 CAR-mast cells incubated with and without HER2-EMT6 cells after 48 hr.

FIGS. 8A and 8B is a line graph and a bar graph respectively, showing cytotoxicity of HER2 CAR-mast cells after 48 hr of coculture with HER2-EMT6 cells at the indicated ratios (FIG. 8A), and against HER2-EMT6 cells in the presence of varying concentrations of Soybean Trypsin Inhibitor (SBTI) (FIG. 8B).

FIG. 9A is a schematic of an in vivo assay during which a FFluc+-HER2-EMT6 engrafted mouse model received one dose (5×10⁶ cells in 100 ul Tyrode's buffer) of CAR-mast cells, WT bone marrow-derived mast cells, or Tyrode's buffer via i.t. injection (n=8 mice per group). FIG. 9B is a graph showing average tumor burden of mice through a 25-day period after FFluc+-HER2-EMT6 cells inoculation. FIG. 9C is a series of plots showing tumor burden of individual mice through a 61-day period post inoculation. Each line represents one mouse. FIG. 9D is a survival curve of mice through a 90-day period post tumor cells inoculation.

FIGS. 10A-10C are bar graphs showing cytotoxicity of HER2 CAR-mast cells determined after 48 hr of coculture with HER2-EMT6 cells (E:T=5:1) (FIG. 10A), and TNFα production (FIG. 10B) and Granzyme B production (FIG. 10C) in the coculture supernatant.

FIG. 11A is a schematic representation of wild-type (HER2 WT) and cytoplasmic domain-truncated HER2 (HER2 dCyto) constructs used for ectopic expression in EMT6 cells. FIGS. 11B and 11C are line graphs showing cytotoxicity (% lysis) and TNFα secretion, respectively, of HER2 CAR-mast cells after 48 hr of coculture with HER2 dCyto-EMT6 cells at the indicated E:T ratios in comparison to CD19 CAR control. FIG. 11D is a bar graph showing CD107 surface level on CAR-mast cells cocultured with HER2 dCyto-EMT6 cells after 48 hr (E:T=5:1). CAR-mast cells were generated from spleen-derived (SPL) mast cells or bone marrow-derived (BM) mast cells.

FIG. 12A is a bar graphs showing lysis rates against HER2-EMT6 cells of supernatant from CAR-mast cells cocultured with HER2-EMT6 cells (E:T=5:1) for 48 hr (“coculture supernatant”), and CAR-mast cells (E:T=5:1) added to a new batch of HER2-EMT6 cells (“cells”). From left to right in clusters for both “cells” and “coculture supernatant” bars correspond to medium (control), HER2 CAR, and CD19 CAR. FIG. 12B shows a schematic transwell setting (left) and results of the assay (right) in which HER2-EMT6 cells were seeded on the insert membrane (3 um pore) and lower bottom of the same transwell. HER 2 CAR-mast cells were added to the insert (E:T=5:1) and cultured for 48 hr. Lysis rates against HER2-EMT6 cells on the insert membrane and lower bottom were determined by luciferase assay. From left to right bars correspond to medium (control), HER2 CAR, and CD19 CAR.

### DETAILED DESCRIPTION OF THE INVENTION

Chimeric Antigen Receptor (CAR)-T cell therapies showed remarkable effects for hematological cancers that are resistant to traditional treatments. However, CARs targeting solid tumors showed limited efficacies. Major challenges include T cells' poor infiltration, low persistence, and exhaustion in a hostile tumor microenvironment. To solve this problem, CAR-mast cells are provided. Mast cells are long-lived and tissue-resident immune cells. When activated by allergens, they release a variety of effectors to induce broad immune reactions. Chimeric antigen receptors (CARs) got mast cells (CAR-mast) that activate mast cells in an antigen-dependent manner are provided. The CAR-mast typically include a mast intracellular signaling domain. The experiments provided herein show CAR-mast cells can specifically kill cancer cells in vitro and release chemokines that recruit T cells and NK cells, e.g., to the tumor tissues and in mouse xenograft models, and inhibit solid tumor growth. Together, these results illustrated CAR-mast cells' efficacy against solid tumors both in vitro and in vivo. As described in more detail below, these results lead to a positive impact on cancer immunology by providing a new strategy for applying CAR therapies to the treatment of cancers including those with solid tumors.

### I. Definitions

“Introduce” in the context of genome modification refers to bringing in to contact. For example, to introduce a gene editing composition to a cell is to provide contact between the cell and the composition. The term encompasses penetration of the contacted composition to the interior of the cell by any suitable means, e.g., via transfection, electroporation, transduction, gene gun, nanoparticle delivery, etc.

As used herein, “homologous” means derived from a common ancestor. For example, a homologous trait is any characteristic of organisms that is inherited by two or more species from a common ancestor species. Homologous sequences can be orthologous or paralogous. Homologous sequences are orthologous if they were separated by a speciation event: when a species diverges into two separate species, the divergent copies of a single gene in the resulting species are said to be orthologous. Orthologs, or orthologous genes, are genes in different species that are similar to each other because they originated from a common ancestor. Homologous sequences are paralogous if they were separated by a gene duplication event: if a gene in an organism is duplicated to occupy two different positions in the same genome, then the two copies are paralogous.

“Heterologous” means having a different relation, relative position, or structure. Thus, unless otherwise specified, heterologous includes joining or linking of two or more amino acid or nucleic acid sequences from that organism (e.g., species) that are not normally found joined or linked (e.g., together) as well as joining or linking of two or more amino acid or nucleic acid sequences from different species.

“Endogenous” refers to any material from or produced inside an organism, cell, tissue or system.

“Exogenous” refers to any material introduced from or produced outside an organism, cell, tissue or system.

The term “transmembrane domain” refers to any protein structure that is thermodynamically stable in a cell membrane, preferably a eukaryotic cell membrane. As used herein, the terms “extracellular domain” and “ectodomain” refer to any protein structure that is thermodynamically stable in outside of the cell membrane (i.e., in the extracellular space). As used herein, an “intracellular domain” refers to any protein structure that is thermodynamically stable in inside of the cell membrane (i.e., in the intracellular cytosol).

The term “Chimeric Antigen Receptor”, or alternatively “CAR”, refers to a set of polypeptides, typically two in the simplest embodiments, which when in an immune effector cell, provides the cell with specificity for a cancer cell, or other specific cell, and with intracellular signal generation. In exemplary embodiments, a CAR includes at least an antigen binding domain such as an extracellular binding domain, a transmembrane domain and a cytoplasmic signaling domain (also referred to as “an intracellular signaling domain”) including a functional signaling domain derived from a stimulatory molecule and/or costimulatory molecule. The term “antigen binding domain” is used in the context of a CAR to refer to the portion of a CAR that specifically recognizes and binds to an antigen of interest. The “antigen binding domain” of a CAR may be derived from a binding protein such as an antibody or fragment thereof. In some embodiments, the “binding domain” of a CAR is a single-chain variable fragment (scFv). In certain embodiments, the “binding domain” of a CAR includes the complementarity determining regions of a binding protein disclosed herein. In some embodiments, the cytoplasmic signaling domain of the CAR further includes one or more functional signaling domains derived from at least one costimulatory molecule. In some embodiments, the CAR includes a chimeric fusion protein including an extracellular antigen binding domain, a transmembrane domain and an intracellular signaling domain including a functional signaling domain derived from a stimulatory molecule. In various embodiments, CARs are fusion proteins of single-chain variable fragments (scFv) fused to a CD3-zeta transmembrane domain.

The term “antigen” as used herein is defined as a molecule capable of being bound by an antibody or T-cell receptor. An antigen can additionally be capable of provoking an immune response. This immune response can involve either antibody production, or the activation of specific immunologically competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which includes a nucleotide sequence or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the disclosed compositions and methods includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid. In the context of cancer, “antigen” refers to an antigenic substance that is produced in a tumor cell, which can therefore trigger an immune response in the host. These cancer antigens can be useful as markers for identifying a tumor cell, which could be a potential candidate/target during treatment or therapy. There are several types of cancer or tumor antigens. There are tumor specific antigens (TSA) which are present only on tumor cells and not on healthy cells, as well as tumor associated antigens (TAA) which are present in tumor cells and on some normal cells. In some forms, the chimeric antigen receptors are specific for tumor specific antigens. In some forms, the chimeric antigen receptors are specific for tumor associated antigens. In some forms, the chimeric antigen receptors are specific both for one or more tumor specific antigens and one or more tumor associated antigens.

The term “immune effector cell,” is used herein to refer to a cell that is involved in an immune response (e.g. promotion of an immune effector response). Examples of immune effector cells include T cells, e.g., alpha/beta T cells and gamma/delta T cells, B cells, natural killer (NK) cells, natural killer T (NKT) cells, mast cells, and myeloic-derived phagocytes. In some embodiments, the immune effector cell(s) is allogenic. In some embodiments, the immune effector cell(s) is autologous. Immune effector cells such as T cells may be activated and expanded generally using methods previously described, such as for example, as described in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041, all of which are incorporated herein by reference in their entirety.

“Bi-specific chimeric antigen receptor” refers to a CAR that includes two domains, wherein the first domain is specific for a first ligand/antigen/target, and wherein the second domain is specific for a second ligand/antigen/target. In some forms, the ligand is a B-cell specific protein, a tumor-specific ligand/antigen/target, a tumor associated ligand/antigen/target, or combinations thereof. A bispecific CAR is specific to two different antigens. A multi-specific or multivalent CAR is specific to more than one different antigen, e.g., 2, 3, 4, 5, or more. In some forms, a multi-specific or multivalent CAR targets and/or binds three or more different antigens.

“Encoding” or “encode” refers to the property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “locus” is the specific physical location of a DNA sequence (e.g., of a gene) on a chromosome. It is understood that a locus of interest can not only qualify a nucleic acid sequence that exists in the main body of genetic material (i.e., in a chromosome) of a cell but also a portion of genetic material that can exist independently to said main body of genetic material such as plasmids, episomes, virus, transposons or in organelles such as mitochondria as non-limiting examples.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell. An “isolated nucleic acid” refers to a nucleic acid segment or fragment which has been separated from sequences which flank it in a naturally occurring state, e.g., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes: a recombinant DNA which is part of a hybrid gene encoding additional polypeptide sequence, complementary DNA (cDNA), linear or circular oligomers or polymers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like.

In the context of cells, the term “isolated” also refers to a cell altered or removed from its natural state. That is, the cell is in an environment different from that in which the cell naturally occurs, e.g., separated from its natural milieu such as by concentrating to a concentration at which it is not found in nature. “Isolated cell” is meant to include cells that are within samples that are substantially enriched for the cell of interest and/or in which the cell of interest is partially or substantially purified.

As used herein, “transformed,” “transduced,” and “transfected” encompass the introduction of a nucleic acid or other material into a cell by one of a number of techniques known in the art.

A “vector” is a composition of matter which includes an isolated nucleic acid and which can be used to deliver the isolated nucleic acid to the interior of a cell. Examples of vectors include but are not limited to, linear polynucleotides, polynucleotides associated with ionic or amphiphilic compounds, plasmids, and viruses. Thus, the term “vector” encompasses an autonomously replicating plasmid or a virus. The term is also construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus (AAV) vectors, retroviral vectors, and the like.

“Tumor burden” or “tumor load” as used herein, refers to the number of cancer cells, the size or mass of a tumor, or the total amount of tumor/cancer in a particular region of a subject. Methods of determining tumor burden for different contexts are known in the art, and the appropriate method can be selected by the skilled person. For example, in some forms, tumor burden can be assessed using guidelines provided in the Response Evaluation Criteria in Solid Tumors (RECIST).

As used herein, “subject” includes, but is not limited to, animals, plants, parasites and any other organism or entity. The subject can be a vertebrate, more specifically a mammal (e.g., a human, horse, pig, rabbit, dog, sheep, goat, non-human primate, cow, cat, guinea pig or rodent), a fish, a bird or a reptile or an amphibian. The subject can be an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects. In some forms, the subject can be any organism in which the disclosed method can be used to genetically modify the organism or cells of the organism.

The term “inhibit” or other forms of the word such as “inhibiting” or “inhibition” means to decrease, hinder or restrain a particular characteristic such as an activity, response, condition, disease, or other biological parameter. It is understood that this is typically in relation to some standard or expected value, i.e., it is relative, but that it is not always necessary for the standard or relative value to be referred to. “Inhibits” can also mean to hinder or restrain the synthesis, expression or function of a protein relative to a standard or control. Inhibition can include, but is not limited to, the complete ablation of the activity, response, condition, or disease. “Inhibits” can also include, for example, a 10% reduction in the activity, response, condition, disease, or other biological parameter as compared to the native or control level. Thus, the reduction can be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%, or any amount of reduction in between as compared to native or control levels. For example, “inhibits expression” means hindering, interfering with or restraining the expression and/or activity of the gene/gene product pathway relative to a standard or a control.

“Treatment” or “treating” means to administer a composition to a subject or a system with an undesired condition (e.g., cancer). The condition can include one or more symptoms of a disease, pathological state, or disorder. Treatment includes medical management of a subject with the intent to cure, ameliorate, stabilize, or prevent a disease, pathological condition, or disorder. This includes active treatment, that is, treatment directed specifically toward the improvement of a disease, pathological state, or disorder, and also includes causal treatment, that is, treatment directed toward removal of the cause of the associated disease, pathological state, or disorder. In addition, this term includes palliative treatment, that is, treatment designed for the relief of symptoms rather than the curing of the disease, pathological state, or disorder; preventative treatment, that is, treatment directed to minimizing or partially or completely inhibiting the development of the associated disease, pathological state, or disorder; and supportive treatment, that is, treatment employed to supplement another specific therapy directed toward the improvement of the associated disease, pathological state, or disorder. It is understood that treatment, while intended to cure, ameliorate, stabilize, or prevent a disease, pathological condition, or disorder, need not actually result in the cure, amelioration, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount. “Prevention” or “preventing” means to administer a composition to a subject or a system at risk for an undesired condition (e.g., cancer). The condition can include one or more symptoms of a disease, pathological state, or disorder. The condition can also be a predisposition to the disease, pathological state, or disorder. The effect of the administration of the composition to the subject can be the cessation of a particular symptom of a condition, a reduction or prevention of the symptoms of a condition, a reduction in the severity of the condition, the complete ablation of the condition, a stabilization or delay of the development or progression of a particular event or characteristic, or reduction of the chances that a particular event or characteristic will occur.

As used herein, the terms “effective amount” or “therapeutically effective amount” means a quantity sufficient to alleviate or ameliorate one or more symptoms of a disorder, disease, or condition being treated, or to otherwise provide a desired pharmacologic and/or physiological effect. Such amelioration only requires a reduction or alteration, not necessarily elimination. The precise quantity will vary according to a variety of factors such as subject-dependent variables (e.g., age, immune system health, weight, etc.), the disease or disorder being treated, as well as the route of administration, and the pharmacokinetics and pharmacodynamics of the agent being administered.

By “pharmaceutically acceptable” is meant a material that is not biologically or otherwise undesirable, i.e., the material can be administered to a subject along with the selected compound without causing any undesirable biological effects or interacting in a deleterious manner with any of the other components of the pharmaceutical composition in which it is contained.

As used herein, the term “polypeptides” includes proteins and functional fragments thereof. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein, the term “functional fragment” or “functional variant” means a fragment or variant of a polypeptide, such as a full-length or native polypeptide, that retains one or more functional properties of the full-length or native polypeptide.

As used herein, the terms “variant” or “active variant” refers to a polypeptide or polynucleotide that differs from a reference polypeptide or polynucleotide, but retains one or more functional properties (e.g., functional or biological activity). A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally. Modifications and changes can be made in the structure of the polypeptides of the disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological or functional activity, certain amino acid sequence substitutions can be made in a polypeptide sequence and nevertheless obtain a polypeptide with like properties (e.g., functional or biological activity).

Modifications and changes can be made in the structure of the polypeptides of in disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly, where the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, and 95% sequence identity to the polypeptide of interest.

As used herein, “conservative” amino acid substitutions are substitutions wherein the substituted amino acid has similar structural or chemical properties.

As used herein, “non-conservative” amino acid substitutions are those in which the charge, hydrophobicity, or bulk of the substituted amino acid is significantly altered.

As used herein, the term “identity,” as known in the art, is a relationship between two or more polypeptide sequences, as determined by comparing the sequences. In the art, “identity” also means the degree of sequence relatedness between polypeptide as determined by the match between strings of such sequences. “Identity” can also mean the degree of sequence relatedness of a polypeptide compared to the full-length of a reference polypeptide. “Identity” and “similarity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48: 1073 (1988).

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48: 443-453, 1970) algorithm (e.g., NBLAST, and XBLAST). The default parameters are used to determine the identity for the polypeptides of the present disclosure.

By way of example, a polypeptide sequence may be identical to the reference sequence, that is be 100% identical, or it may include up to a certain integer number of amino acid alterations as compared to the reference sequence such that the % identity is less than 100%. Such alterations are selected from: at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the reference polypeptide by the numerical percent of the respective percent identity (divided by 100) and then subtracting that product from said total number of amino acids in the reference polypeptide.

It is to be understood that the disclosed method and compositions are not limited to specific synthetic methods, specific analytical techniques, or to particular reagents unless otherwise specified, and, as such, can vary. It is also to be understood that the terminology used herein is for the purpose of describing particular embodiments only and is not intended to be limiting.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout the description and claims of this specification, the word “comprise” and variations of the word, such as “comprising” and “comprises,” means “including but not limited to,” and is not intended to exclude, for example, other additives, components, integers or steps.

“Optional” or “optionally” means that the subsequently described event, circumstance, or material may or may not occur or be present, and that the description includes instances where the event, circumstance, or material occurs or is present and instances where it does not occur or is not present.

The disclosed method and compositions can be understood more readily by reference to the following detailed description of particular embodiments and the Example included therein and to the Figures and their previous and following description. Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a fusion protein is disclosed and discussed and a number of modifications that can be made to a number of molecules including the fusion protein are discussed, each and every combination and permutation of the fusion protein and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials. These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Unless the context clearly indicates otherwise, use of the word “can” indicates an option or capability of the object or condition referred to. Generally, use of “can” in this way is meant to positively state the option or capability while also leaving open that the option or capability could be absent in other forms or embodiments of the object or condition referred to. Unless the context clearly indicates otherwise, use of the word “may” indicates an option or capability of the object or condition referred to. Generally, use of “may” in this way is meant to positively state the option or capability while also leaving open that the option or capability could be absent in other forms or embodiments of the object or condition referred to. Unless the context clearly indicates otherwise, use of “may” herein does not refer to an unknown or doubtful feature of an object or condition.

Ranges can be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. It should be understood that all of the individual values and sub-ranges of values contained within an explicitly disclosed range are also specifically contemplated and should be considered disclosed unless the context specifically indicates otherwise. Finally, it should be understood that all ranges refer both to the recited range as a range and as a collection of individual numbers from and including the first endpoint to and including the second endpoint. In the latter case, it should be understood that any of the individual numbers can be selected as one form of the quantity, value, or feature to which the range refers. In this way, a range describes a set of numbers or values from and including the first endpoint to and including the second endpoint from which a single member of the set (i.e. a single number) can be selected as the quantity, value, or feature to which the range refers. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

### II. Compositions

Mast cells are a type of tissue-resident granulocytes that have a lifespan of up to years. They are recognized for their large quantity of intracellular granules. A wide range of mediators are stored in these granules including proteases, cytotoxic factors, chemokines, cytokines, and bioactive chemicals¹³. These granules can be rapidly released into the extracellular space, a process called degranulation, upon the stimuli from allergens and pathogens^{14,15}. After granule release, intriguingly, mast cells can refill the granules and undergo repetitive release for multiple rounds¹⁶. Functionally, mast cells are best-known for their roles in triggering allergic inflammation and in defending parasite infection^{17,18}.

Meanwhile, the role of mast cells in cancer progression is much less characterized. Correlation studies revealed that mast cells have either positive, negative, or no roles in predicting patient outcomes and these observations are highly tumor-type specific¹⁹⁻²⁴ Recently, a cause-and-effect relationship has been established between mast cells and solid tumors: adoptively transferred mast cells, when primed by LPS or IgE, inhibit tumor growth in xenograft models of melanoma and breast cancer^{25,26}, indicating an anti-tumor function of mast cells.

The experiments disclosed herein exemplify mast cells as a new effector cell for CAR. Mast cells are advantageous as effector cells because (1) they release cytotoxic factors including granzyme B and TNFα that induce target cell deaths²⁷⁻²⁹, (2) they release chemokines and cytokines including CCL3, CCL4, and CXCL10 that recruit T cells and NK cells into the tumor and remodel TME^{25,30,31}, (3) they are long-lived (up to years) in tissues, release mediators repeatedly^{32,33}, and could confer a sustainable anti-tumor effect, and (4) do not express conventional inhibitory receptors including PD-1, CTLA-4, TIM-3, or LAG-3 at either resting or activated states³⁴ and are thus not susceptible to these immune suppressive signals in the TME.

The experiments establish that engineered Chimeric Antigen Receptor (CAR) polypeptides having a mast intracellular signaling domain can activate mast cells. Fusion proteins including, but not limited to CAR polypeptides incorporating a mast cell intracellular signaling domain together with one or more heterologous sequences are provided. Recombinant constructs including nucleic acids expressing or encoding the polypeptides. Viral genomes including the recombinant constructs, recombinant viruses including the constructs, and vaccine formulations formed thereof are also provided, as are cells, particularly mast cells, including the nucleic acids and/or the fusion polypeptides are also provided.

The disclosed compositions and methods are especially applicable to development of chimeric antigen receptor engineered mast cell therapy (CAR-mast).

### A. Mast Cell Intracellular Signaling Domains

Compositions typically include or encode a polypeptide including mast cell intracellular signaling domain gene product or variant thereof, referred to herein as “mast cell intracellular signaling domain polypeptides”, are provided. Typically, the mast cell intracellular signaling domain polypeptide is between about 10 amino acids and about 500 amino acids, or any specific integer number of amino acids therebetween, including, but not limited to 20, 25, 30, 35, 40, 45, 60, 70, 80, 90, 100, 125, 150, 175, 200, 225, 250, 275, 300, 350, 400, or 450 amino acids; preferably, at least a length that is sufficient to promote intracellular signal transduction in mast cells, preferably leading to enhanced mast cell activation when forming a fusion protein with CAR. The mast cell intracellular signaling domain polypeptides, nucleic acids encoding the same, and delivery vehicles thereof, and cells including them can optionally include one or more additional heterologous proteins, polypeptides, or other amino acid sequences. Typically, the presence of one or more mast cell intracellular signaling domain polypeptides within the cytoplasmic domain of a recombinant fusion peptide will impart an increased cell activation and/or signaling to/from the fusion peptide. Thus, as discussed in more below, typically, mast cell intracellular signaling domain polypeptides are described as part of a fusion protein.

The native receptors that activate mast cells include the high affinity IgE receptor (FcεRI), cytokine receptors (e.g., c-KIT), G-protein-coupled receptors, and toll-like receptors (Gilfillan, and Tkaczyk, Nat Rev Immunol 6:218-230 (2006)). Thus, the mast intracellular signaling domain can be or include the intracellular domain of any of IgE receptor (FcRI), cytokine receptors (e.g., c-KIT), G-protein-coupled receptors, and toll-like receptors (e.g., TLR2, TLR4, TLR6, etc.), preferably ones that are expressed by mast cells.

Among those, FcεRI is of particular interest because 1) the pathway by which FcεRI triggers degranulation is well characterized and 2) FcRI contains the immunoreceptor tyrosine-based activation motif (ITAM). ITAM is important for the signal transduction in CAR T cells, and thus, the mast cell intracellular signaling domain can include a ITAM sequence.

An exemplary ITAM sequence from human FcRI is

(SEQ ID NO: 11)
DGVYTGLSTRNQETYETLKHE.

In an exemplary embodiment utilized in the experiments below, the mast cell intracellular domain is the intracellular domain from mouse FcεR1gamma:

(SEQ ID NO: 12)
RLKIQVRKAAIASREKADAVYTGLNTRSQETYETLKHEKPPQ,

with the ITAM sequence in bold and italics.

However, preferably, the domains of a CAR for use in human cells include mast cell intracellular signaling domain from a human protein or fragment or variant thereof.

In the case of SEQ ID NO:12, the corresponding segment of a human FcεR1gamma intracellular domain protein is

(SEQ ID NO: 13)
RLKIQVRKAAITSYEKSDGVYTGLSTRNQETYETLKHEKPPQ,

with the ITAM sequence in bold and italics.

See, e.g., NCBI Reference Sequence: NP_004097.1, UniProt Ref No.:P30273-FCERG_HUMAN, and UniProt Ref No: P20491⋅FCERG_MOUSE each of which is specifically incorporated by references herein in its entirety.

In particular embodiments, the mast intracellular signaling domain is or includes a sequence from C-kit, TLR2, TLR4, or TLR6, e.g., mouse or human C-kit, TLR2, TLR4, or TLR6, preferably including or being the intracellular domain thereof or a functional fragment or variant thereof.

In particular embodiments, the mast intracellular signaling domain is or includes the intracellular domain or a functional fragment or variant thereof, of:

UniProt Ref No. P05532⋅KIT_MOUSE, which is specifically incorporated by reference in its entirety, and provides an intracellular domain having the amino acid sequence of

(SEQ ID NO: 14)
KYLQKPMYEVQWKVVEEINGNNYVYIDPTQLPYDHKWEFPRNRLSFGKTL
GAGAFGKVVEATAYGLIKSDAAMTVAVKMLKPSAHLTEREALMSELKVLS
YLGNHMNIVNLLGACTVGGPTLVITEYCCYGDLLNFLRRKRDSFIFSKQE
EQAEAALYKNLLHSTEPSCDSSNEYMDMKPGVSYVVPTKTDKRRSARIDS
YIERDVTPAIMEDDELALDLDDLLSFSYQVAKGMAFLASKNCIHRDLAAR
NILLTHGRITKICDFGLARDIRNDSNYVVKGNARLPVKWMAPESIFSCVY
TFESDVWSYGIFLWELFSLGSSPYPGMPVDSKFYKMIKEGFRMVSPEHAP
AEMYDVMKTCWDADPLKRPTFKQVVQLIEKQISDSTKHIYSNLANCNPNP
ENPVVVDHSVRVNSVGSSASSTQPLLVHEDA;

UniProt Ref No. P10721⋅KIT_HUMAN, which is specifically incorporated by reference in its entirety, and provides an intracellular domain having the amino acid sequence of

(SEQ ID NO: 15)
KYLQKPMYEVQWKVVEEINGNNYVYIDPTQLPYDHKWEFPRNRLSFGKTL
GAGAFGKVVEATAYGLIKSDAAMTVAVKMLKPSAHLTEREALMSELKVLS
YLGNHMNIVNLLGACTIGGPTLVITEYCCYGDLLNFLRRKRDSFICSKQE
DHAEAALYKNLLHSKESSCSDSTNEYMDMKPGVSYVVPTKADKRRSVRIG
SYIERDVTPAIMEDDELALDLEDLLSFSYQVAKGMAFLASKNCIHRDLAA
RNILLTHGRITKICDFGLARDIKNDSNYVVKGNARLPVKWMAPESIFNCV
YTFESDVWSYGIFLWELFSLGSSPYPGMPVDSKFYKMIKEGFRMLSPEHA
PAEMYDIMKTCWDADPLKRPTFKQIVQLIEKQISESTNHIYSNLANCSPN
RQKPVVDHSVRINSVGSTASSSQPLLVHDDV;

UniProt Ref No. Q9QUN7⋅TLR2_MOUSE, which is specifically incorporated by reference in its entirety, and provides an intracellular domain having the amino acid sequence of

(SEQ ID NO: 16)
CHHFHGLWYLRMMWAWLQAKRKPKKAPCRDVCYDAFVSYSEQDSHWVENL
MVQQLENSDPPFKLCLHKRDFVPGKWIIDNIIDSIEKSHKTVFVLSENFV
RSEWCKYELDFSHFRLFDENNDAAILVLLEPIERKAIPQRFCKLRKIMNT
KTYLEWPLDEGQQEVFWVNLRTAIKS;

UniProt Ref No. O60603⋅TLR2_HUMAN, which is specifically incorporated by reference in its entirety, and provides an intracellular domain having the amino acid sequence of

(SEQ ID NO: 17)
HRFHGLWYMKMMWAWLQAKRKPRKAPSRNICYDAFVSYSERDAYWVENLM
VQELENFNPPFKLCLHKRDFIPGKWIIDNIIDSIEKSHKTVFVLSENFVK
SEWCKYELDFSHFRLFDENNDAAILILLEPIEKKAIPQRFCKLRKIMNTK
TYLEWPMDEAQREGFWVNLRAAIKS;

UniProt Ref No. Q9QUK6⋅TLR4_MOUSE, which is specifically incorporated by reference in its entirety, and provides an intracellular domain having the amino acid sequence of

(SEQ ID NO: 18)
AGCKKYSRGESIYDAFVIYSSQNEDWVRNELVKNLEEGVPRFHLCLHYRD
FIPGVAIAANIIQEGFHKSRKVIVVVSRHFIQSRWCIFEYEIAQTWQFLS
SRSGIIFIVLEKVEKSLLRQQVELYRLLSRNTYLEWEDNPLGRHIFWRRL
KNALLDGKASNPEQTAEEEQETATWT;

UniProt Ref No. O00206⋅TLR4_HUMAN, which is specifically incorporated by reference in its entirety, and provides an intracellular domain having the amino acid sequence of

(SEQ ID NO: 19)
KFYFHLMLLAGCIKYGRGENIYDAFVIYSSQDEDWVRNELVKNLEEGVPP
FQLCLHYRDFIPGVAIAANIIHEGFHKSRKVIVVVSQHFIQSRWCIFEYE
IAQTWQFLSSRAGIIFIVLQKVEKTLLRQQVELYRLLSRNTYLEWEDSVL
GRHIFWRRLRKALLDGKSWNPEGTVGTGCNWQEATSI;

UniProt Ref No. Q9EPW9⋅TLR6_MOUSE, which is specifically incorporated by reference in its entirety, and provides an intracellular domain having the amino acid sequence of

(SEQ ID NO: 20)
CLYFDLPWYVRMLCQWTQTRHRARHIPLEELQRNLQFHAFVSYSEHDSAW
VKNELLPNLEKDDIRVCLHERNFVPGKSIVENIINFIEKSYKAIFVLSPH
FIQSEWCHYELYFAHHNLFHEGSDNLILILLEPILQNNIPSRYHKLRALM
AQRTYLEWPTEKGKRGLFWANLRASFIMKLALVNEDDVKT;

UniProt Ref No. Q9Y2C9⋅TLR6_HUMAN, which is specifically incorporated by reference in its entirety, and provides an intracellular domain having the amino acid sequence of

(SEQ ID NO: 21)
YLDLPWYLRMVCQWTQTRRRARNIPLEELQRNLQFHAFISYSEHDSAWVK
SELVPYLEKEDIQICLHERNFVPGKSIVENIINCIEKSYKSIFVLSPNFV
QSEWCHYELYFAHHNLFHEGSNNLILILLEPIPQNSIPNKYHKLKALMTQ
RTYLQWPKEKSKRGLFWANIRAAFNMKLTLVTENNDVKS.

The compositions typically are, or include, a mast intracellular signaling domain or a functional fragment or variant thereof, or a nucleic acid encoding the same. Functional fragments and variants can be, for example, any number of amino acids sufficient to drive enhanced mast cell activation. The data below supports the conclusions that CAR-mast enhances proinflammatory cytokine secretion and tumor cell killing by CAR-mast cells having a CAR-mast fusion protein.

In some embodiments, the mast intracellular signaling domain is between about 20 amino acids and about 500 amino acids, or any specific integer number of amino acids therebetween. Variants can have, for example, at least 65%, 70%, 75%, 80%, 85%, 90%, 95%, 99% or 100% sequence identity to any one of the provided SEQ ID NOs, or a functional fragment thereof; or the corresponding sequence of a homologue such as an orthologue or paralogue of any of the foregoing sequences; or any combination thereof. Preferably variants maintain the ability to promote intracellular signaling in a mast cell. In some embodiments, variants are identified as functional in a CAR fusion construct if they maintain or enhance cytotoxicity of a host mast cell in vitro or in vivo towards target cells, expression one or more inflammatory cytokines and/or chemokines such as those discussed in the experiments below, recruitment or attraction of T and/or NK cells e.g., into tumor tissues, or a combination thereof.

Any of the polypeptide sequences including the amino acid sequences of any of the SEQ ID NOs provided anywhere herein can include one or more amino acid substitutions. Typically, the amino acid substitutions do not reduce the ability of the polypeptide in fusion construct to promote cell activation and/or signaling, for example in the case of CAR-mast fusion proteins maintain or enhance cytotoxicity of a host mast cell in vitro or in vivo towards target cells, expression one or more inflammatory cytokines and/or chemokines such as those discussed in the experiments below, recruitment or attraction of T and/or NK cells e.g., into tumor tissues, or a combination thereof. Amino acid substitutions within peptides are generally based on the relative similarity of the amino acid side-chain substituents, for example, their hydrophobicity, hydrophilicity, charge, size, and the like. Exemplary substitutions of amino acids within any of the provided SEQ ID NOs can include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of the provided polypeptides, e.g., as set forth in any of the disclosed SEQ ID NOs.

### B. Fusion Peptides

Fusion proteins, including one or more heterologous polypeptide sequences fused to one or more mast cell intracellular signaling polypeptides are provided.

The term “mast cell intracellular signaling domain” is used in the context of fusion peptides that include one or more signaling domains, to refer to the component of the fusion peptide that includes a mast cell intracellular signaling domain.

In some embodiments, in addition to a mast cell intracellular signaling domain, the fusion proteins include one or more extracellular polypeptide domains, and/or one or more transmembrane domains, and/or one or more additional (i.e., heterologous) intracellular domain.

### 1. Heterologous Sequences

Heterologous elements that can be associated with, linked, conjugated, or otherwise attached directly or indirectly to the mast cell intracellular signaling domain sequence(s), or nucleic acids expressing the mast cell intracellular signaling domain are disclosed. Such molecules include, but are not limited to, protein domains, such as transduction domains, fusogenic peptides, targeting molecules, and sequences that enhance protein expression and/or isolation. Suitable protein domains include ectodomains, transmembrane domains, cytoplasmic domains of proteins and macromolecular structures including combinations of ectodomains, transmembrane domains, and cytoplasmic domains. Typically, the other protein domains are not proteins from which the mast cell intracellular signaling domain is derived. In some embodiments, the other protein domains have or have potential for one or more molecular functions or activities. Such “functional” domains can be engineered to provide one or more functions or activities, as desired. Exemplary functions include receptor or ligand binding, enzymic activity, and molecular transport, such as active transport of one or more molecules into or out of one or more cellular compartments. In some embodiments, the other protein domains within a mast cell intracellular signaling domain fusion protein bind to a specific substrate or molecule. An exemplary molecule is an antigen or a cell-surface receptor.

Thus, in some embodiments, mast cell intracellular signaling domain fusion peptides include one or more heterologous peptide domains, such as receptors at the surface of a cell, optionally including a transmembrane domain that anchors or connects the ectodomain to the cell surface and connects with the intracellular signaling domain.

Exemplary cell surface receptors coordinate the activity of cells upon interaction with other cells, such as immune cells, such as mast cells, T cells, etc. For example, in some embodiments, the heterologous domain is a recombinant or engineered chimeric antigen receptor (CAR). In other embodiments, the heterologous domain includes a specific transmembrane domain (e.g., transmembrane domain of CD8 or CD28) for further enhancing signaling and receptor sensitivity in the case of CAR. In further embodiments, the heterologous domain is a co-signaling domain. Exemplary co-signaling domains have been previously described (Majzner, R. G. et al., Cancer Discov, doi:10.1158/2159-8290.CD-19-0945 (2020); Heitzeneder, S. et al., Cancer Cell 40, 53-69 e59, (2022); Priceman, S. J. et al., Oncoimmunology 7, e1380764, (2018)) In some embodiments, the fusion peptides include multiple heterologous domains, such as a CAR domain (e.g., an antigen binding domain such as a single chain variable fragment targeting a cancer antigen, e.g., CD19, GD2, B7H3, or others mentioned or exemplified herein), a stalk and/or transmembrane domain (e.g., of CD8α), or a combination thereof.

Various domains of the fusion protein can optionally be linked with a linker. Exemplary linkers include, but are not limited to,

a. Chimeric Antigen Receptors (CAR)

In some forms, the fusion protein includes a Chimeric Antigen Receptor (CAR) fused with one or more mast cell intracellular signaling domains (“CAR-mast”). Typically, CARs include an extracellular domain, a transmembrane domain and one or more intracellular/cytoplasmic domains. Typically, the mast cell intracellular signaling domain forms part or all of the intracellular signaling domain of the CAR-mast.

Therefore, in some embodiments, a CAR-mast fusion protein includes the extracellular and transmembrane domains of an existing CAR fused to one or more mast intracellular signaling domains alone or in further combination with one or more additional intracellular domains.

i. CAR Structure

CARs are engineered receptors that possess both antigen-binding and cell-activating functions. Immunotherapy using cells genetically engineered to express a CAR is rapidly emerging as a promising new treatment for hematological and non-hematological malignancies. Based on the location of the CAR in the membrane of the cell, the CAR can be divided into three main distinct domains, including an extracellular antigen-binding domain, followed by a space region, a transmembrane domain, and the intracellular signaling domain. The antigen-binding domain, most commonly derived from variable regions of immunoglobulins, typically contains VH and VL chains that are joined up by a linker to form the so-called “scFv.” The segment interposing between the antigen-binding domain (e.g., scFv) and the transmembrane domain is a “spacer domain.” The spacer domain can include the constant IgG1 hinge-CH2-CH3 Fc domain. In some cases, the spacer domain and the transmembrane domain are derived from CD8. The traditional CARs, the intracellular signaling domains mediating T cell activation can include a CD3ζ co-receptor signaling domain derived from C-region of the TCR α and β chains and one or more costimulatory domains. In the disclosed CAR-mast constructs, a mast intracellular signaling domain typically supplements or replaces one or more, or all, of the intracellular domains of a traditional CAR.

In some forms, the antigen-binding domain is derived from an antibody. The term antibody herein refers to natural or synthetic polypeptides that bind a target antigen. The term includes polyclonal and monoclonal antibodies, including intact antibodies and functional (e.g., antigen-binding) antibody fragments, including Fab fragments, F(ab′)₂ fragments, Fab′ fragments, Fv fragments, recombinant IgG (rlgG) fragments, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. The term also encompasses intact or full-length antibodies, including antibodies of any class or subclass, including IgG and sub-classes thereof, IgM, IgE, IgA, and IgD. The antigen-binding domain of a CAR can contain complementary determining regions (CDR) of an antibody, variable regions of an antibody, and/or antigen binding fragments thereof. For example, the antigen-binding domain for a CD19 CAR can be derived from a human monoclonal antibody to CD19, such as those described in U.S. Pat. No. 7,109,304, which is specifically incorporated by reference herein in its entirety for use in accordance with the disclosed compositions and methods. In some forms, the antigen-binding domain can include an F(ab′)2, Fab′, Fab, Fv or scFv.

In some forms, the CAR includes one or more spacer domain(s) (also referred to as hinge domain) that is located between the extracellular antigen-binding domain and the transmembrane domain. A spacer domain is an amino acid segment that is generally found between two domains of a protein and may allow for positioning and flexibility of the protein and movement of one or both of the domains relative to one another. Any amino acid sequence that provides such positioning, flexibility and movement of the extracellular antigen-binding domain relative to the transmembrane domain can be used. The spacer domain can be a spacer or hinge domain of a naturally occurring protein. In some forms, the hinge domain is derived from CD8α or CD28, such as, a portion of the hinge domain of CD8α or CD28, e.g., a fragment containing at least 5 (e.g., 5, 10, 15, 20, 25, 30, 35, or 40) consecutive amino acids of the hinge domain of CD8α. Hinge domains of antibodies, such as an IgG, IgA, IgM, IgE, or IgD antibodies can also be used. In some forms, the hinge domain is the hinge domain that joins the constant CH1 and CH2 domains of an antibody. Non-naturally occurring peptides may also be used as spacer domains. For example, the spacer domain can be a peptide linker, such as a (GxS)n linker, wherein x and n, independently can be an integer of 3 or more, including 3, 4, 5, 6, 7, 8, 9, 10, 11, 12, or more.

In some forms, the CAR includes a transmembrane domain that can be directly or indirectly fused to the antigen-binding domain. The transmembrane domain may be derived either from a natural or a synthetic source. In some forms, the transmembrane domain of the CAR includes a transmembrane domain of an alpha, beta, or zeta chain of a T-cell receptor, CD8, CD4, CD28, CD137, CD80, CD86, CD152 (CTLA-4) or PD1, or a portion thereof. Transmembrane domains can also contain at least a portion of a synthetic, non-naturally occurring protein segment. In some forms, the transmembrane domain is a synthetic, non-naturally occurring alpha helix or beta sheet. In some forms, the protein segment is at least about 15 amino acids, e.g., at least 15, 16, 17, 18, 19, 20, 21, 22, 23, 24, 25, 26, 27, 28, 29, 30, or more amino acids. Examples of synthetic transmembrane domains are known in the art, for example in U.S. Pat. No. 7,052,906 and PCT Publication No. WO 2000/032776.

The CAR-mast constructs in the experiments below utilize as the transmembrane:

Human CD8α transmembrane domain:
(SEQ ID NO: 4)
IYIWAPLAGTCGVLLLSLVITLYCR;
or
Mouse CD28 stalk and transmembrane domain:
(SEQ ID NO: 10)
IEVMYPPPYLDNEKSNGTIIHVKGKHLCPSPLFPGPSKPFWVLVVVGGVL
ACYSLLVTVAFIIFWV;

either of which can be utilized as the antigen binding domain in any of the disclosed CAR-mast constructs.

Preferably, the domains of a CAR for use in human cells includes a human protein or fragment or variant thereof. In the case of SEQ ID NO:10, the corresponding segment of a human CD28 stalk and transmembrane domain protein is 100% identical to SEQ ID NO:10, see, e.g., NCBI Reference Sequence: NP_006130.1.

The intracellular signaling domain is responsible for activation of at least one of the normal effector functions of the immune effector cell expressing the CAR. The term effector function refers to a specialized function of a cell. Effector function of a mast cell, for example, may be cytotoxicity of the mast cell in vitro or in vivo towards target cells, expression one or more inflammatory cytokines and/or chemokines such as those discussed in the experiments below, recruitment or attraction T and/or NK cells e.g., into tumor tissues, or a combination thereof. The mast cell intracellular signaling domains are discussed in more detail above, and thus not repeated here.

In some forms, an intracellular signaling domain includes the zeta chain of the T cell receptor or any of its homologs (e.g., eta, delta, gamma, or epsilon), MB1 chain, B29, Fc RIII, Fc RI and combinations of signaling molecules such as CD3ζ and CD28, 4-1BB, OX40 and combination thereof, as well as other similar molecules and fragments. Intracellular signaling portions of other members of the families of activating proteins can be used, such as FcγRIII and FcεRI.

Many immune effector cells require co-stimulation, in addition to stimulation of an antigen-specific signal, to promote cell proliferation, differentiation and survival, as well as to activate effector functions of the cell. Therefore, in some forms, the CAR includes at least one co-stimulatory signaling domain. The term co-stimulatory signaling domain, refers to at least a portion of a protein that mediates signal transduction within a cell to induce an immune response such as an effector function. The co-stimulatory signaling domain can be a cytoplasmic signaling domain from a co-stimulatory protein, which transduces a signal and modulates responses mediated by immune cells, such as T cells, NK cells, macrophages, neutrophils, or eosinophils. In some forms, the co-stimulatory signaling domain is derived from a co-stimulatory molecule selected from TLR2, TLR4, TLR6, IL3, FceR1b, c-kit, CD27, CD28, CD137, 0X40, CD30, CD40, CD3, LFA-1, ICOS, CD2, CD7, LIGHT, NKG2C, B7-H3, ligands of CD83 and combinations thereof. In one form, the co-stimulatory signaling domain is derived from CD28.

CARs can be used in order to generate immuno-responsive cells, such as mast cells, specific for selected targets, such as malignant cells. A wide variety of receptor chimera constructs having been described (see e.g., U.S. Pat. Nos. 5,843,728; 5,851,828; 5,912,170; 6,004,811; 6,284,240; 6,392,013; 6,410,014; 6,753,162; 8,211,422; and PCT Publication WO 9215322, each of which is specifically incorporated by reference herein in its entirety), and their component parts and domains (e.g., the extracellular domain and transmembrane domain in particular), can be combined with the disclosed mast cell intracellular domains to form CAR-mast constructs.

Alternative CAR constructs can be characterized as belonging to successive generations. First-generation CARs typically include a single-chain variable fragment of an antibody specific for an antigen, for example including a VL linked to a VH of a specific antibody, linked by a flexible linker, for example by a CD8α hinge domain and a CD8α transmembrane domain, to the transmembrane and intracellular signaling domains of either CD3ζ or FcRγ (scFv-CD3ζ or scFv-FcRγ; see U.S. Pat. Nos. 7,741,465; 5,912,172; 5,906,936, each of which is specifically incorporated by reference herein in its entirety).

Second-generation CARs incorporate the intracellular domains of one or more costimulatory molecules, such as CD28, OX40 (CD134), or 4-1BB (CD137) within the endodomain (for example scFv-CD28/OX40/4-1BB-CD3; see U.S. Pat. Nos. 8,911,993; 8,916,381; 8,975,071; 9,101,584; 9,102,760; 9,102,761, each of which is specifically incorporated by reference herein in its entirety).

Third-generation CARs include a combination of costimulatory endodomains, such a CD3ζ-chain, CD97, GDI 1a-CD18, CD2, ICOS, CD27, CD154, CDS, OX40, 4-1BB, or CD28 signaling domains (for example scFv-CD28-4-1BB-CD3ζ or scFv-CD28-OX40-CD3ζ; see U.S. Pat. Nos. 8,906,682; 8,399,645; 5,686,281; PCT Publication No. WO2014134165; PCT Publication No. WO2012079000, each of which is specifically incorporated by reference herein in its entirety). Any of the first, second, or third generation CARs described above can be modified in accordance with the disclosed compositions and methods, e.g., to supplement or replace the intracellular signaling domain of such CARs with a mast intracellular signaling domain as provided herein, to form a CAR-mast.

ii. Exemplary Antigens and CAR Embodiments

The target specificity of the cell expressing a CAR is determined by the antigen recognized by the antibody/ectodomain. The disclosed compositions and methods can be used to create constructs, and cells expressing the constructs, that target any antigen. In the context of immunotherapy, particularly cancer immunotherapy, numerous antigens, and suitable ectodomains for targeting them, are well known. Unlike the native TCR, the majority of scFv-based CARs recognize target antigens expressed on the cell surface rather than internal antigens that are processed and presented by the cells' MHC, however, CARs have the advantage over the classical TCR that they can recognize structures other than protein epitopes, including carbohydrates and glycolipids Dotti, et al., Immunol Rev. 2014 January; 257(1):. doi:10.1111/imr.12131 (35 pages) thus increasing the pool of potential target antigens. Preferred targets include antigens that are only expressed on cancer cells or their surrounding stroma (Cheever, et al., Clin Cancer Res., 15:5323-5337 (2009)), such as the splice variant of EGFR (EGFRvIII), which is specific to glioma cells (Sampson, et al., Semin Immunol., 20(5):267-75 (2008)). However, human antigens meet this requirement, and the majority of target antigens are expressed either at low levels on normal cells (e.g. GD2, CAIX, HER2) and/or in a lineage restricted fashion (e.g. CD19, CD20).

Preferred targets, and CARs that target them are known in the art (see, e.g., Dotti, et al., Immunol Rev. 2014 January; 257(1):. doi:10.1111/imr.12131 (35 pages). For example, CAR targets for hematological malignancies include, but are not limited to, CD 19 (e.g., B-cell) (Savoldo, et al., J Clin Invest., 121:1822-1826 (2011), Cooper, et al., Blood, 105:1622-1631 (2005); Jensen, et al., Biol Blood Marrow Transplant (2010), Kochenderfer, et al., Blood, 119:2709-2720 (2012), Brentjens, et al., Molecular Therapy, 17:S157 (2009), Brentjens, et al., Nat Med., 9:279-286 (2003), Brentjens, et al., Blood, 118:4817-4828 (2011), Porter, et al., N Engl J Med., 365:725-733 (2011), Kalos, et al., Sci Transl Med., 3:95ra73 (2011), Brentjens, et al., Sci Transl Med., 5:177ra38 (2013), Grupp, et al., N Engl J Med (2013)); CD20 (e.g., B-cell) (Jensen, et al., Biol Blood Marrow Transplant (2010), Till, et al., Blood, 112:2261-2271 (2008), Wang, et al., Hum Gene Ther., 18:712-725 (2007), Wang, et al., Mol Ther., 9:577-586 (2004), Jensen, et al., Biol Blood Marrow Transplant, 4:75-83 (1998)); CD22 (e.g., B-cell) (Haso, et al., Blood, 121:1165-1174 (2013)); CD30 (e.g., B-cell) (Di Stasi, et al., Blood, 113:6392-6402 (2009), Savoldo, et al., Blood, 110:2620-2630 (2007), Hombach, et al., Cancer Res., 58:1116-1119 (1998)); CD33 (e.g., Myeloid) (Finney, et al., J Immunol., 161:2791-2797 (1998)); CD70 (e.g., B-cell/T-cell) (Shaffer, et al., Blood, 117:4304-4314 (2011)); CD123 (e.g., Myeloid) (Tettamanti, et al., Br J Haematol., 161:389-401 (2013)); Kappa (e.g., B-cell) (Vera, et al., Blood, 108:3890-3897 (2006)); Lewis Y (e.g., Myeloid) (Peinert, et al., Gene Ther., 17:678-686 (2010), Ritchie, et al., Mol Ther. (2013)); NKG2D ligands (e.g., Myeloid) (Barber, et al., Exp Hematol., 36:1318-1328 (2008), Lehner, et al., PLoS One., 7:e31210 (2012), Song, et al., Hum Gene Ther., 24:295-305 (2013), Spear, et al., J Immunol. 188:6389-6398 (2012)); ROR1 (e.g., B-cell) (Hudecek, et al., Clin Cancer Res. (2013)).

CAR targets for solid tumors include, but are not limited to, B7H3 (e.g., sarcoma, glioma) (Cheung, et al., Hybrid Hybridomics, 22:209-218 (2003)); CAIX (e.g., kidney) (Lamers, et al., J Clin Oncol., 24:e20-e22. (2006)), Weijtens, et al., Int J Cancer, 77:181-187 (1998)); CD44 v6/v7 (e.g., cervical) (Hekele, et al., Int J Cancer, 68:232-238 (1996)), Dall, et al., Cancer Immunol Immunother, 54:51-60 (2005); CD171 (e.g., neuroblastoma) (Park, et al., Mol Ther., 15:825-833 (2007)); CEA (e.g., colon) (Nolan, et al., Clin Cancer Res., 5:3928-3941 (1999)); EGFRvIII (e.g., glioma) (Bullain, et al., J Neurooncol. (2009), Morgan, et al., Hum Gene Ther., 23:1043-1053 (2012)); EGP2 (e.g., carcinomas) (Meier, et al., Magn Reson Med., 65:756-763 (2011), Ren-Heidenreich, et al., Cancer Immunol Immunother., 51:417-423 (2002)); EGP40 (e.g., colon) (Daly, et al., Cancer Gene Ther., 7:284-291 (2000); EphA2 (e.g., glioma, lung) (Chow, et al., Mol Ther., 21:629-637 (2013)); ErbB2(HER2) (e.g., breast, lung, prostate, glioma) (Zhao, et al., J Immunol., 183:5563-5574 (2009), Morgan, et al., Mol Ther., 18:843-851 (2010), Pinthus, et al., 114:1774-1781 (2004), Teng, et al., Hum Gene Ther., 15:699-708 (2004), Stancovski, et al., J Immunol., 151:6577-6582 (1993), Ahmed, et al., Mol Ther., 17:1779-1787 (2009), Ahmed, et al., Clin Cancer Res., 16:474-485 (2010), Moritz, et al., Proc Natl Acad Sci U.S.A., 91:4318-4322 (1994)); ErbB receptor family (e.g., breast, lung, prostate, glioma) (Davies, et al., Mol Med., 18:565-576 (2012)); ErbB3/4 (e.g., breast, ovarian) (Muniappan, et al., Cancer Gene Ther., 7:128-134 (2000), Altenschmidt, et al., Clin Cancer Res., 2:1001-1008 (1996)); HLA-A1/MAGE1 (e.g., melanoma) (Willemsen, et al., Gene Ther., 8:1601-1608 (2001), Willemsen, et al., J Immunol., 174:7853-7858 (2005)); HLA-A2/NY-ESO-1 (e.g., sarcoma, melanoma) (Schuberth, et al., Gene Ther., 20:386-395 (2013)); FR-α (e.g., ovarian) (Hwu, et al., J Exp Med., 178:361-366 (1993), Kershaw, et al., Nat Biotechnol., 20:1221-1227 (2002), Kershaw, et al., Clin Cancer Res., 12:6106-6115 (2006), Hwu, et al., Cancer Res., 55:3369-3373 (1995)); FAP (e.g., cancer associated fibroblasts) (Kakarla, et al., Mol Ther. (2013)); FAR (e.g., rhabdomyosarcoma) (Gattenlohner, et al., Cancer Res., 66:24-28 (2006)); GD2 (e.g., neuroblastoma, sarcoma, melanoma) (Pule, et al., Nat Med., 14:1264-1270 (2008), Louis, et al., Blood, 118:6050-6056 (2011), Rossig, et al., Int J Cancer., 94:228-236 (2001)); GD3 (e.g., melanoma, lung cancer) (Yun, et al., Neoplasia., 2:449-459 (2000)); HMW-MAA (e.g., melanoma) (Burns, et al., Cancer Res., 70:3027-3033 (2010)); IL11Rα (e.g., osteosarcoma) (Huang, et al., Cancer Res., 72:271-281 (2012)); IL13Rα2 (e.g., glioma) (Kahlon, et al., Cancer Res., 64:9160-9166 (2004), Brown, et al., Clin Cancer Res. (2012), Kong, et al., Clin Cancer Res., 18:5949-5960 (2012), Yaghoubi, et al., Nat Clin Pract Oncol., 6:53-58 (2009)); Lewis Y (e.g., breast/ovarian/pancreatic) (Peinert, et al., Gene Ther., 17:678-686 (2010), Westwood, et al., Proc Natl Acad Sci U.S.A., 102:19051-19056 (2005), Mezzanzanica, et al., Cancer Gene Ther., 5:401-407 (1998)); Mesothelin (e.g., mesothelioma, breast, pancreas) (Lanitis, et al., Mol Ther., 20:633-643 (2012), Moon, et al., Clin Cancer Res., 17:4719-4730 (2011)); Muel (e.g., ovarian, breast, prostate) (Wilkie, et al., J Immunol., 180:4901-4909 (2008)); NCAM (e.g., neuroblastoma, colorectal) (Gilham, et al., J Immunother., 25:139-151 (2002)); NKG2D ligands (e.g., ovarian, sacoma) (Barber, et al., Exp Hematol., 36:1318-1328 (2008), Lehner, et al., PLoS One, 7:e31210 (2012), Song, et al., Gene Ther., 24:295-305 (2013), Spear, et al., J Immunol., 188:6389-6398 (2012)); PSCA (e.g., prostate, pancreatic) (Morgenroth, et al., Prostate, 67:1121-1131 (2007), Katari, et al., HPB, 13:643-650 (2011)); PSMA (e.g., prostate) (Maher, et al., Nat Biotechnol., 20:70-75 (2002), Gong, et al., Neoplasia., 1:123-127 (1999)); TAG72 (e.g., colon) (Hombach, et al., Gastroenterology, 113:1163-1170 (1997), McGuinness, et al., Hum Gene Ther., 10:165-173 (1999)); VEGFR-2 (e.g., tumor vasculature) (J Clin Invest., 120:3953-3968 (2010), Niederman, et al., Proc Natl Acad Sci U.S.A., 99:7009-7014 (2002)).

In some forms, the CAR targeting one or more antigens specific for cancer, an inflammatory disease, a neuronal disorder, HIV/AIDS, diabetes, a cardiovascular disease, an infectious disease, an autoimmune disease, or combinations thereof. One of skill in the art, based on general knowledge in the field and/or routine experimentation would be able to determine the appropriate antigen to be targeted by a CAR for a specific disease, disorder, or condition.

Exemplary antigens specific for cancer that could be targeted by the CAR include, but are not limited to, 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD 152, CD 19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNT0888, B7H3, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, L1-CAM, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, vimentin, and combinations thereof.

In preferred forms, the CAR targets CD19, GD2, or B7H3. The CAR-mast constructs in the experiments below utilize as the antigen binding domain:

Human CD19 single chain Fv
(SEQ ID NO: 2)
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEITGGGGSGGGGSGGGGSEVKLQESGPGLVAPSQSLSVTCTVSGVS
LPDYGVSWIRQPPRKGLEWLGVIWGSETTYYNSALKSRLTIIKDNSKSQV
FLKMNSLQTDDTAIYYCAKHYYYGGSYAMDYWGQGTSVTVSS,
GD2 single chain Fv:
(SEQ ID NO: 8)
HPAFLLIPQVQLVESGPGVVQPGRSLRISCAVSGFSVTNYGVHWVRQPPG
KGLEWLGVIWAGGITNYNSAFMSRLTISKDNSKNTVYLQMNSLRAEDTAM
YYCASRGGHYGYALDYWGQGTLVTVSSGSTSGSGKPGSSEGSTKGEIVMT
QTPATLSVSAGERVTITCKASQSVSNDVTWYQQKPGQAPRLLIYSASNRY
SGVPARFSGSGYGTEFTFTISSVQSEDFAVYFCQQDYSSFGQGTKLEIKT
S;
Human B7H3 single chain Fv:
(SEQ ID NO: 9)
QVKLQQSGAELVKPGASVKLSCKASGYTFTNYDINWVRQRPEQGLEWIGW
IFPGDGSTQYNEKFKGKATLTTDTSSSTAYMQLSRLTSEDSAVYFCARQT
TATWFAYWGQGTTVTVSSDGGGSGGGGSGGGGSDIELTQSPTTLSVTPGD
RVSLSCRASQSISDYLHWYQQKSHESPRLLIKYASQSISGIPSRFSGSGS
GSDFTLSINSVEPEDVGVYYCQNGHSFPLTFGAGTKLELKQTS;
or
HER2 single chain Fv:
(SEQ ID NO: 34)
KYLLPTAAAGLLLLAAQPAMAQVQLVQSGAEVKKPGESLKISCKGSGYSF
TSYWIAWVRQMPGKGLEYMGLIYPGDSDTKYSPSFQGQVTISVDKSVSTA
YLQWSSLKPSDSAVYFCARHDVGYCTDRTCAKWPEYFQHWGQGTLVTVSS
GGGGSGGGGSGGGGSQSVLTQPPSVSAAPGQKVTISCSGSSSNIGNNYVS
WYQQLPGTAPKLLIYDHTNRPAGVPDRFSGSKSGTSASLAISGFRSEDEA
DYYCASWDYTLSGWVFGGGTKLTVLGGSGS,

any of which can be utilized as the antigen binding domain in any of the disclosed CAR-mast constructs.
   b. Other Protein Domains

Any of the disclosed recombinant proteins can include one or more additional domains. For example, any of the disclosed proteins can include one or more linkers or spacers. The term “linker” as used herein includes, without limitation, peptide linkers. The peptide linker can be any size provided it does not interfere with the binding of the epitope by the variable regions. In some embodiments, the linker includes one or more glycine and/or serine amino acid residues. In some embodiments, the linker includes a glycine-glutamic acid di-amino acid sequence. For example, a linker can include 4-8 amino acids. In a particular embodiment, a linker includes the amino acid sequence GQSSRSS (SEQ ID NO:25). In some forms, the linker includes one, two or more copies the amino acid sequence GGGGS (SEQ ID NO:26). In another embodiment, a linker includes 15-20 amino acids, for example 18 amino acids. Other flexible linkers include, but are not limited to, the amino acid sequences Gly-Ser, Gly-Ser-Gly-Ser (SEQ ID NO:27), Ala-Ser, Gly-Gly-Gly-Ser (SEQ ID NO:28), (Gly₄-Ser)₂ (SEQ ID NO:29) and (Gly₄-Ser)₄ (SEQ ID NO:30), (Gly-Gly-Gly-Ser)₂ (SEQ ID NO: 31) and (Gly-Gly-Gly-Gly-Ser)₃ (SEQ ID NO:32).

The linkers can be used to link or connect two domains, regions, or sequences of a fusion protein. Molecular biology techniques have developed so that therapeutic proteins can be genetically engineered to be expressed by microorganisms.

In some embodiments, the compositions disclosed herein include one or more of a signal peptide, a marker, and/or expression or solubility enhancing amino acid sequence. An exemplary signal peptide is provided in the examples below, but can be substituted of another signal peptide as is known in the art. Exemplary markers are include GFP, mCherry and HA, each of which is exemplified in the experiments below. Exemplary expression or solubility enhancing amino acid sequences include maltose-binding protein (MBP), glutathione S-transferase (GST), thioredoxin (TRX), NUS A, ubiquitin (Ub), and a small ubiquitin-related modifier (SUMO). In some embodiments, the signal peptide, marker, and/or expression or solubility enhancing amino acid sequence is cleaved during or after post-translation processing.

### C. Nucleic Acids

Nucleic acids and vectors encoding or expressing the disclosed polypeptides and fusion proteins are also described.

### 1. Isolated Nucleic Acid Molecules

Isolated nucleic acid sequences encoding mast intracellular signaling domain polypeptides and fusion peptides are disclosed. In some embodiments, the isolated nucleic acid sequences encode a CAR-mast. Thus, nucleic acid encoding the disclosed SEQ ID NOs. alone or in any combination are expressly provided.

The term “isolated nucleic acid” refers to a nucleic acid that is separated from other nucleic acid molecules that are present in a mammalian genome, including nucleic acids that normally flank one or both sides of the nucleic acid in a mammalian genome. An isolated nucleic acid can be, for example, a DNA molecule, provided one of the nucleic acid sequences normally found immediately flanking that DNA molecule in a naturally occurring genome is removed or absent. Thus, an isolated nucleic acid includes, without limitation, a DNA molecule that exists as a separate molecule independent of other sequences (e.g., a chemically synthesized nucleic acid, or a cDNA or genomic DNA fragment produced by PCR or restriction endonuclease treatment), as well as recombinant DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a recombinant DNA molecule that is part of a hybrid or fusion nucleic acid. A nucleic acid existing among hundreds to millions of other nucleic acids within, for example, a cDNA library or a genomic library, or a gel slice containing a genomic DNA restriction digest, is not to be considered an isolated nucleic acid.

Nucleic acids can be in sense or antisense orientation, or can be complementary to a reference sequence a polypeptide or fusion peptide. Thus, nucleic acids encoding the provided polypeptides (e.g., the disclosed amino acid sequence SEQ ID NOs), and fragments and variants thereof, in sense and antisense, and in single stranded and double stranded forms, are provided. The nucleic acids can be DNA, RNA, or nucleic acid analogs. Nucleic acid analogs can be modified at the base moiety, sugar moiety, or phosphate backbone. Such modification can improve, for example, stability, hybridization, or solubility of the nucleic acid. Modifications at the base moiety can include deoxyuridine for deoxythymidine, and 5-methyl-2′-deoxycytidine or 5-bromo-2′-deoxycytidine for deoxycytidine. Modifications of the sugar moiety can include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained. See, for example, Summerton and Weller (1997) Antisense Nucleic Acid Drug Dev. 7:187-195; and Hyrup et al. (1996) Bioorgan. Med. Chen. 4:5-23. In addition, the deoxyphosphate backbone can be replaced with, for example, a phosphorothioate or phosphorodithioate backbone, a phosphoroamidite, or an alkyl phosphotriester backbone.

### 2. Vectors

In some embodiments, nucleic acids encoding the provided polypeptides and fusion proteins are present within vectors. In some embodiments, the vectors encode or express a CAR-mast. Vector encoding or expressing any one or more of the disclosed polypeptide sequence (e.g., disclosed SEQ ID NOs) are also expressly provided.

Vectors including an isolated polynucleotide encoding a polypeptide or fusion protein for the expression of the polypeptide or fusion peptide within a host cell are described.

The term “vector” is a nucleic acid molecule used to carry genetic material into another cell, where it can be replicated and/or expressed. Any vector known to those skilled in the art in view of the present disclosure can be used. Examples of vectors include, but are not limited to, plasmids, viral vectors (bacteriophage, animal viruses, and plant viruses), cosmids, and artificial chromosomes (e.g., YACs). A vector can be a DNA vector or an RNA vector. In some embodiments, a vector is a DNA plasmid. One of ordinary skill in the art can construct a vector of the application through standard recombinant techniques in view of the present disclosure.

In some embodiments, the vector including nucleic acids encoding a polypeptide or fusion protein is an expression vector. The term “expression vector” refers to any type of genetic construct including a nucleic acid coding for an RNA capable of being transcribed. Expression vectors include, but are not limited to, vectors for recombinant protein expression, such as a DNA plasmid or a viral vector, and vectors for delivery of nucleic acid into a subject for expression in a tissue of the subject, such as a DNA plasmid or a viral vector. It will be appreciated by those skilled in the art that the design of the expression vector can depend on such factors as the choice of the host cell to be transformed, the level of expression of protein desired, etc.

In some embodiments, vectors contain one or more regulatory sequences. The term “regulatory sequence” refers to any sequence that allows, contributes or modulates the functional regulation of the nucleic acid molecule, including replication, duplication, transcription, splicing, translation, stability and/or transport of the nucleic acid or one of its derivative (i.e. mRNA) into the host cell or organism. In the context of the disclosure, this term encompasses promoters, enhancers and other expression control elements (e.g., polyadenylation signals and elements that affect mRNA stability).

In some embodiments, the vector is a non-viral vector. Examples of non-viral vectors include, but are not limited to, DNA plasmids, bacterial artificial chromosomes, yeast artificial chromosomes, bacteriophages, etc. Examples of non-viral vectors include, but are not limited to, RNA replicon, mRNA replicon, modified mRNA replicon or self-amplifying mRNA, closed linear deoxyribonucleic acid, e.g., a linear covalently closed DNA, e.g., a linear covalently closed double stranded DNA molecule. Preferably, a non-viral vector is a DNA plasmid. A “DNA plasmid”, which is used interchangeably with “DNA plasmid vector,” “plasmid DNA” or “plasmid DNA vector,” refers to a double-stranded and generally circular DNA sequence that is capable of autonomous replication in a suitable host cell. DNA plasmids used for expression of an encoded polynucleotide typically include an origin of replication, a multiple cloning site, and a selectable marker, which for example, can be an antibiotic resistance gene. Examples of suitable DNA plasmids that can be used include, but are not limited to, commercially available expression vectors for use in well-known expression systems (including both prokaryotic and eukaryotic systems), such as pSE420 (Invitrogen, San Diego, Calif.), which can be used for production and/or expression of protein in Escherichia coli; pYES2 (Invitrogen, Thermo Fisher Scientific), which can be used for production and/or expression in Saccharomyces cerevisiae strains of yeast; MAXBAC®. complete baculovirus expression system (Thermo Fisher Scientific), which can be used for production and/or expression in insect cells; pcDNA™. or pcDNA3™ (Life Technologies, Thermo Fisher Scientific), which can be used for high level constitutive protein expression in mammalian cells; and pVAX or pVAX-1 (Life Technologies, Thermo Fisher Scientific), which can be used for high-level transient expression of a protein of interest in most mammalian cells. The backbone of any commercially available DNA plasmid can be modified to optimize protein expression in the host cell, such as to reverse the orientation of certain elements (e.g., origin of replication and/or antibiotic resistance cassette), replace a promoter endogenous to the plasmid (e.g., the promoter in the antibiotic resistance cassette), and/or replace the polynucleotide sequence encoding transcribed proteins (e.g., the coding sequence of the antibiotic resistance gene), by using routine techniques and readily available starting materials. (See e.g., Sambrook et al., Molecular Cloning a Laboratory Manual, Second Ed. Cold Spring Harbor Press (1989)).

Preferably, a DNA plasmid is an expression vector suitable for protein expression in mammalian host cells. Expression vectors suitable for protein expression in mammalian host cells include, but are not limited to, pcDNA™, pcDNA3™, pVAX, pVAX-1, ADVAX, NTC8454, etc. In some embodiments, an expression vector is based on pVAX-1, which can be further modified to optimize protein expression in mammalian cells. pVAX-1 is a commonly used plasmid in DNA vaccines, and contains a strong human immediate early cytomegalovirus (CMV-IE) promoter followed by the bovine growth hormone (bGH)-derived polyadenylation sequence (pA). pVAX-1 further contains a pUC origin of replication and a kanamycin resistance gene driven by a small prokaryotic promoter that allows for bacterial plasmid propagation.

In some embodiments, the vector is a viral vector. In general, viral vectors are genetically engineered viruses carrying modified viral DNA or RNA that has been rendered non-infectious, but still contains viral promoters and transgenes, thus allowing for translation of the transgene through a viral promoter. Because viral vectors are frequently lacking infectious sequences, they require helper viruses or packaging lines for large-scale transfection. Examples of viral vectors that can be used include, but are not limited to, adenoviral vectors, adeno-associated virus vectors, pox virus vectors, enteric virus vectors, Venezuelan Equine Encephalitis virus vectors, Semliki Forest Virus vectors, Tobacco Mosaic Virus vectors, lentiviral vectors, arenavirus viral vectors, replication-deficient arenavirus viral vectors or replication-competent arenavirus viral vectors, bi-segmented or tri-segmented arenavirus, infectious arenavirus viral vectors, nucleic acids which include an arenavirus genomic segment wherein one open reading frame of the genomic segment is deleted or functionally inactivated and replaced by a nucleic acid encoding a heterologous polypeptide or fusion protein as described herein), arenavirus such as lymphocytic choriomeningitidis virus (LCMV), e.g., clone 13 strain or MP strain, and arenavirus such as Junin virus e.g., Candid #1 strain, etc.

In some embodiments, the viral vector is an adenovirus vector, e.g., a recombinant adenovirus vector. A recombinant adenovirus vector can for instance be derived from a human adenovirus (HAdV, or AdHu), or a simian adenovirus such as chimpanzee or gorilla adenovirus (ChAd, AdCh, or SAdV) or rhesus adenovirus (rhAd). Preferably, an adenovirus vector is a recombinant human adenovirus vector, for instance a recombinant human adenovirus serotype 26, or any one of recombinant human adenovirus serotype 5, 4, 35, 7, 48, etc. In other embodiments, an adenovirus vector is a rhAd vector, e.g., rhAd51, rhAd52 or rhAd53. In some embodiments, a recombinant viral vector is prepared using methods known in the art in view of the present disclosure. For example, in view of the degeneracy of the genetic code, several nucleic acid sequences can be designed that encode the same polypeptide. In some embodiments, a polynucleotide encoding a provided polypeptide or fusion polypeptide is codon-optimized to ensure proper expression in the host cell (e.g., bacterial or mammalian cells). Codon-optimization is a technology widely applied in the art, and methods for obtaining codon-optimized polynucleotides will be well known to those skilled in the art in view of the present disclosure.

In some embodiments, the vectors, e.g., a DNA plasmid or a viral vector (particularly an adenoviral vector), include any regulatory elements to establish conventional function(s) of the vector, including but not limited to replication and expression of the polypeptide or fusion protein encoded by the polynucleotide sequence of the vector.

### 3. Regulatory Elements

In some embodiments, the disclosed nucleic acids, including RNAs and DNAs such as DNA vectors expressing or encoding the provided polypeptides and fusion proteins include one or more regulatory elements.

Regulatory elements include, but are not limited to, a promoter, an enhancer, a polyadenylation signal, translation stop codon, a ribosome binding element, a transcription terminator, selection markers, origin of replication, etc. An isolated nucleic acid can be, and a vector can include one or more expression cassettes. An “expression cassette” is part of a nucleic acid such as a vector that directs the cellular machinery to make RNA and protein. An expression cassette typically includes three components: a promoter sequence, an open reading frame, and a 3-untranslated region (UTR) optionally including a polyadenylation signal. An open reading frame (ORF) is a reading frame that contains a coding sequence of a protein of from a start codon to a stop codon. Regulatory elements of the expression cassette can be operably linked to a polynucleotide sequence encoding polypeptide or fusion protein.

As used herein, the term “operably linked” is to be taken in its broadest reasonable context, and refers to a linkage of polynucleotide (or polypeptide, etc.) elements in a functional relationship. A polynucleotide is “operably linked” when it is placed into a functional relationship with another polynucleotide. For instance, a promoter is operably linked to a coding sequence if it affects the transcription of the coding sequence. Any components suitable for use in an expression cassette described herein can be used in any combination and in any order to prepare vectors of the application.

a. Promotors

The disclosed nucleic acids, including vectors, can include a promoter sequence, preferably within an expression cassette, to control expression of a polypeptide or fusion polypeptide. The term “promoter” is used in its conventional sense and refers to a nucleotide sequence that initiates the transcription of an operably linked nucleotide sequence. A promoter is located on the same strand near the nucleotide sequence it transcribes. Promoters can be a constitutive, inducible, or repressible. Promoters can be naturally occurring or synthetic. A promoter can be derived from sources including viral, bacterial, fungal, plants, insects, and animals. A promoter can be a homologous promoter (i.e., derived from the same genetic source as the vector) or a heterologous promoter (i.e., derived from a different vector or genetic source). For example, if the vector to be employed is a DNA plasmid, the promoter can be endogenous to the plasmid (homologous) or derived from other sources (heterologous). Preferably, the promoter is located upstream of the polynucleotide encoding an a protein of interest within an expression cassette.

Examples of promoters that can be used include, but are not limited to, a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter (CMV-IE), Epstein Barr virus (EBV) promoter, or a Rous sarcoma virus (RSV) promoter. A promoter can also be a promoter from a human gene such as human actin, human myosin, human hemoglobin, human muscle creatine, or human metallothionein.

A promoter can also be a tissue specific promoter, such as a kidney specific promoter, preferably a kidney epithelial cell promoter, which can be natural or synthetic. Examples include, but are not limited to, the CDH 16 promoter, which is mostly kidney specific (it is also expressed in the thyroid) (Igarashi, et al., Am J Physiol., 277(4):F599-610 (1999)); the Pax-8 promoter, which is also expressed primarily in the kidney as well as in the thyroid (Dehbi, et al., EMBO J., 15(16):4297-306 (1996)); the aquaporin 2 promoter, which drives expression specifically in principal cells of the renal collecting duct (which are the target of Tolvaptan) (Stricklett, et al., Exp Nephrol., 7(1):67-74 (1999)), and kidney tubule-specific promoters in association with gene delivery viral vectors (Watanabe, et al., PloS one, vol. 12,3 e0168638 (2017)).

In some embodiments, the promoter is a strong eukaryotic promoter, such as cytomegalovirus immediate early (CMV-IE) promoter.

b. Other Expression Control Elements

In some embodiments, the nucleic acids, including vectors, include additional polynucleotide sequences that stabilize the expressed transcript, enhance nuclear export of the RNA transcript, and/or improve transcriptional-translational coupling. Examples of such sequences include polyadenylation signals and enhancer sequences. A polyadenylation signal is typically located downstream of the coding sequence for a disclosed polypeptide or fusion protein within an expression cassette of the vector. Enhancer sequences are regulatory DNA sequences that, when bound by transcription factors, enhance the transcription of an associated gene. An enhancer sequence is preferably located upstream of the polynucleotide sequence encoding polypeptide or fusion protein, but downstream of a promoter sequence within an expression cassette of the vector.

Any polyadenylation signal known to those skilled in the art in view of the present disclosure can be used. For example, the polyadenylation signal can be a SV40 polyadenylation signal, LTR polyadenylation signal, bovine growth hormone (bGH) polyadenylation signal, human growth hormone (hGH) polyadenylation signal, or human beta-globin polyadenylation signal. Preferably, a polyadenylation signal is a bovine growth hormone (bGH) polyadenylation signal or a SV40 polyadenylation signal.

Any enhancer sequence known to those skilled in the art in view of the present disclosure can be used. For example, an enhancer sequence can be a human actin, human myosin, human hemoglobin, human muscle creatine, or a viral enhancer, such as one from CMV, HA, RSV, or EBV. Examples of particular enhancers include, but are not limited to, Woodchuck HBV Post-transcriptional regulatory element (WPRE), intron/exon sequence derived from human apolipoprotein A1 precursor (ApoAI), untranslated R-U5 domain of the human T-cell leukemia virus type 1 (HTLV-1) long terminal repeat (LTR), a splicing enhancer, a synthetic rabbit beta-globin intron, or any combination thereof. Preferably, an enhancer sequence is a composite sequence of three consecutive elements of the untranslated R-U5 domain of HTLV-1 LTR, rabbit beta-globin intron, and a splicing enhancer, which is referred to herein as “a triple enhancer sequence.”

A vector can include a polynucleotide sequence encoding a signal peptide sequence. Preferably, the polynucleotide sequence encoding the signal peptide sequence is located upstream of the polynucleotide sequence encoding polypeptide or fusion protein. Signal peptides typically direct localization of a protein, facilitate secretion of the protein from the cell in which it is produced, and/or improve expression the therapeutic polypeptide when expressed from the vector, but is cleaved off by signal peptidase, e.g., upon secretion from the cell. An expressed protein in which a signal peptide has been cleaved is often referred to as the “mature protein.” Any signal peptide known in the art in view of the present disclosure can be used. For example, a signal peptide can be a cystatin S signal peptide; an immunoglobulin (Ig) secretion signal, such as the Ig heavy chain gamma signal peptide SPIgG or the Ig heavy chain epsilon signal peptide SPIgE.

A vector, such as a DNA plasmid, can also include a bacterial origin of replication and an antibiotic resistance expression cassette for selection and maintenance of the plasmid in bacterial cells, e.g., E. coli. Bacterial origins of replication and antibiotic resistance cassettes can be located in a vector in the same orientation as the expression cassette encoding a polypeptide or fusion protein, or in the opposite (reverse) orientation. An origin of replication (ORI) is a sequence at which replication is initiated, enabling a plasmid to reproduce and survive within cells. Examples of ORIs suitable for use in the application include, but are not limited to ColE1, pMB1, pUC, pSC101, R6K, and 15A, preferably pUC.

Expression cassettes for selection and maintenance in bacterial cells typically include a promoter sequence operably linked to an antibiotic resistance gene. Preferably, the promoter sequence operably linked to an antibiotic resistance gene differs from the promoter sequence operably linked to a polynucleotide sequence encoding a protein of interest. The antibiotic resistance gene can be codon optimized, and the sequence composition of the antibiotic resistance gene is normally adjusted to bacterial, e.g., E. coli, codon usage. Any antibiotic resistance gene known to those skilled in the art in view of the present disclosure can be used, including, but not limited to, kanamycin resistance gene (Kan^{r}), ampicillin resistance gene (Amp^{r}), and tetracycline resistance gene (Tet^{r}), as well as genes conferring resistance to chloramphenicol, bleomycin, spectinomycin, carbenicillin, etc.

An expression vector can include a tag sequence, such as those discussed above.

### D. Host Cells

In some embodiments, polypeptides, nucleic acids, or vectors encoding the disclosed polypeptides or fusion proteins are present within a host cells. In some embodiments, the cells include nucleic acids or vectors or genes that encode or express a CAR-mast. The term “host cell” is intended to include prokaryotic and eukaryotic cells into which a recombinant expression vector can be introduced. As used herein, “transformed” and “transfected” encompass the introduction of a nucleic acid molecule (e.g., a vector) into a cell by one of a number of techniques. Although not limited to a particular technique, a number of these techniques are well established within the art. Prokaryotic cells can be transformed with nucleic acids by, for example, electroporation or calcium chloride mediated transformation. Nucleic acids can be transfected into mammalian cells by techniques including, for example, calcium phosphate co-precipitation, DEAE-dextran-mediated transfection, lipofection, electroporation, or microinjection. Host cells (e.g., a prokaryotic cell or a eukaryotic cell) can be used to, for example, produce the polypeptides and fusions proteins described herein.

In some forms, the cell is from an established cell line, or a primary cell. The term “primary cell,” refers to cells and cell cultures derived from a subject and allowed to grow in vitro for a limited number of passages, i.e., splitting, of the culture.

In some embodiments, cells are obtained from a human subject. Therefore, human cells expressing and/or including disclosed polypeptides and fusion proteins are described. In preferred embodiments, the human cells include or express a CAR-mast. For example, in some forms, the cells are autologous cells, i.e., cells obtained from a subject prior to introduction of the disclosed polypeptides or fusion proteins such as a CAR-mast, and/or nucleic acids, or vectors encoding the same, and re-introduction to the same subject following modification. In other forms, the cells are heterologous cells, i.e., cells obtained from a different subject than the intended recipient. In some forms, the cells are frozen prior to or after introduction of the disclosed polypeptides or fusion proteins such as a CAR-mast, and/or nucleic acids, or vectors encoding the same. Methods and compositions for freezing and thawing viable eukaryotic cells are known in the art. In some forms, the cells are autologous immune cells, such as mast cells or progenitor cells/stem cells.

In some forms, cells are obtained from a healthy subject. In other forms, cells are obtained from a subject identified as having or at risk of having a disease or disorder, such as cancer and/or an auto-immune disease.

In preferred embodiments, the introduction of the disclosed polypeptides or fusion proteins such as a CAR-mast to the cells occurs through genetic modification of the cells. In some embodiments, genetic modification of the cell includes introduction of nucleic acids, or vectors encoding the disclosed polypeptides or fusion proteins such as a CAR-mast within the cell. In some embodiments, genetic modification of the cell includes transduction with a transposon encoding a disclosed polypeptide or fusion protein such as a CAR-mast. In an exemplary embodiment, a CAR-mast fusion peptide is introduced into a cell in vitro by transduction of the cell with a nucleic acid encoding a transposon including the CAR-mast. Therefore, genetically modified (transgenic) cells including CAR-mast and/or other mast intracellular signaling domain-fusion protein(s) according to the described compositions are described.

In some forms, the cells are human immune cells, such as mast cells. Therefore, human mast cells that include or express the disclosed polypeptides or fusion proteins such as a CAR-mast are described. In some forms, prior to expansion and genetic modification, cells are obtained from a diseased or healthy subject.

Mast cells can be obtained from a number of samples, including peripheral blood and cord blood, adipose tissue, skin, and/or can be induced from progenitor cells. In some forms, mast cells are obtained from a unit of blood collected from a subject using any number of techniques known to the skilled artisan.

Mast cells are derived from CD34+ hematopoietic progenitor cells that are defined as KIT (CD117)+ and CD13+, but FcεRI− cells (Kirshenbaum et al. Blood. 1999; 94:2333-2342). Undifferentiated mast cell progenitors leave the bone marrow and undergo their terminal differentiation in tissues; consequently, very few mature mast cells are found circulating in the peripheral blood. Even in their resident tissues, mast cells are only found in limited numbers, thus, primary human mast cells are difficult to isolate. An alternative means of obtaining reasonable populations of primary human mast cells has been by in vitro differentiation of CD34+ progenitors. CD34+ peripheral blood-derived human mast cells have the appearance of mature human mast cells having a well condensed non-lobate nucleus and abundant granules in their cytosol. They express FcεRI, KIT, and various G protein-coupled receptors (GPCRs), and respond, through these receptors to promote degranulation and cytokine production or, in the case of KIT or specific GPCRs in conjunction with FcεRI, to synergistically enhance these responses (Gilfillan et al, Nat. Rev. Immunol. 2006; 6:218-230, Kuehn et al., Immunol. Lett. 2007; 113:59-69). A number of toll-like receptors (TLRs) are also expressed on mast cells and, when activated, have the capacity to enhance antigen-mediated cytokine production (Qiao et al., Blood. 2006; 107:610-618).

Cord blood contains a higher concentration of progenitors than does peripheral blood, which makes it a convenient progenitor source for mast cell generation. Both mononuclear cells and purified CD34+ and CD133+ progenitors have been used.

See, e.g., Radinger, et al., Curr Protoc Immunol. 2010 August; CHAPTER: Unit-7.37, which is specifically incorporated by reference herein in its entirety.

Mast cells expressing a CAR-mast can be referred to as CAR-mast cells.

### E. Delivery Vehicles

Any of the disclosed compositions including, but not limited to the provided polypeptides, fusion proteins, and/or nucleic acids encoding the same, can be delivered to target cells using a delivery vehicle. The delivery vehicles can be, for example, polymeric particles, inorganic particles, silica particles, liposomes, micelles, multilamellar vesicles, etc.

Delivery vehicles may be microparticles or nanoparticles. Nanoparticles are often utilized for intertissue application, penetration of cells, and certain routes of administration. The nanoparticles may have any desired size for the intended use. The nanoparticles may have any diameter from 10 nm up to about 1,000 nm. The nanoparticle can have a diameter from 10 nm to 900 nm, from 10 nm to 800 nm, from 10 nm to 700 nm, from 10 nm to 600 nm, from 10 nm to 500 nm, from 20 nm from 500 nm, from 30 nm to 500 nm, from 40 nm to 500 nm, from 50 nm to 500 nm, from 50 nm to 400 nm, from 50 nm to 350 nm, from 50 nm to 300 nm, or from 50 nm to 200 nm. In some embodiments the nanoparticles can have a diameter less than 400 nm, less than 300 nm, or less than 200 nm. The range can be between 50 nm and 300 nm.

Thus, in some embodiments, the delivery vehicles are nanoscale compositions, for example, 10 nm up to, but not including, about 1 micron. However, it will be appreciated that in some embodiments, and for some uses, the particles can be smaller, or larger (e.g., microparticles, etc.). Although many of the compositions disclosed herein are referred to as nanoparticle or nanocarrier compositions, it will be appreciated that in some embodiments and for some uses the carrier can be somewhat larger than nanoparticles. Such compositions can be referred to as microparticulate compositions. For example, a nanocarriers according to the present disclosure may be a microparticle. Microparticles can a diameter between, for example, 0.1 and 100 μm in size.

### F. Pharmaceutical Compositions

Pharmaceutical compositions containing nucleic acids encoding the provided polypeptides and fusions, or a genetically modified cell, or a population of genetically modified cells expressing the disclosed polypeptides and fusion proteins such as a CAR-mast are also provided.

In some embodiments, the pharmaceutical compositions include one or more of a pharmaceutically acceptable buffer, carrier, diluent, or excipients. In some forms, the pharmaceutical compositions include a specific number or population of cells, for example, expanded by culturing and expanding an isolated genetically modified cell (e.g., CAR-mast cell), e.g., a homogenous population. Therefore, in some embodiments, pharmaceutical compositions include a homogenous population of modified cells, e.g., mast cells, including and/or expressing a disclosed polypeptide or fusion protein, such as a CAR-mast. In other forms, the pharmaceutical compositions include populations of cells that contain variable or different genetically modified cells, e.g., a heterogeneous population. In some forms, the pharmaceutical compositions include cells that are bispecific or multi-specific. In some embodiments, the cells have been isolated from a diseased or healthy subject prior to genetic modification to express the polypeptide or fusion protein, such as a CAR-mast.

The term “pharmaceutically acceptable carrier” describes a pharmaceutically acceptable material, composition, or vehicle that is involved in carrying or transporting a compound of interest from one tissue, organ, or portion of the body to another tissue, organ, or portion of the body. For example, in some forms the carrier is a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

In some embodiments, pharmaceutical compositions include buffers such as neutral buffered saline, phosphate buffered saline and the like; carbohydrates such as glucose, mannose, sucrose or dextran, mannitol; proteins; polypeptides or amino acids such as glycine; antioxidants; chelating agents such as EDTA or glutathione; adjuvants (e.g., aluminum hydroxide); and preservatives. The pharmaceutical compositions can be formulated for delivery via any route of administration. The term “Route of administration” can refer to any administration pathway known in the art, including but not limited to aerosol, nasal, oral, intravenous, intramuscular, intraperitoneal, inhalation, transmucosal, transdermal, parenteral, implantable pump, continuous infusion, topical application, capsules and/or injections. The pharmaceutical compositions are preferably formulated for intravenous administration.

Typically, the disclosed pharmaceutical compositions are administered in a manner appropriate to a disease to be treated (or prevented). The quantity and frequency of administration is typically determined by such factors as the condition of the patient, and the type and severity of the patient's disease, although appropriate dosages can be determined by clinical trials.

The disclosed pharmaceutical compositions can be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

### III. Methods

Methods of using the disclosed compositions including, but not limited to, polypeptides and fusions proteins such as CAR-mast and cells expressing the same, e.g., CAR-mast cells, are provided.

In some embodiments, the methods enhance the efficacy of cell receptor-mediated functions are provided. In particular embodiments, the methods provide enhanced anti-tumor activity through administration of CAR-mast cells including CAR-mast fusion peptides.

As set forth in the Examples below, the disclosed CAR-mast constructs can be used to maintain or enhance cytotoxicity of a host mast cell towards target cells, maintain or enhance expression one or more inflammatory cytokines and/or chemokines such as those discussed in the experiments below, to recruit or attract T and/or NK cells e.g., into tumor tissues, or a combination thereof.

Together, the disclose provides a distinct approach to engineer CAR-mast cells with functional reprogramming via addition of one or more mast intracellular signaling domains. The enhanced CAR-mast function is independent of CAR types (i.e., antigen target) and (ii) can be applied to a broad range of cell therapies including targeting of both blood and solid tumors. Furthermore, based on the modular feature of CAR, mast intracellular signaling domains can be combined with other engineering strategies for improving antigen sensitivity, including selecting specific transmembrane or co-signaling domains (Majzner, R. G. et al., Cancer Discov, doi:10.1158/2159-8290.CD-19-0945 (2020); Heitzeneder, S. et al., Cancer Cell 40, 53-69 e59, (2022); Priceman, S. J. et al., Oncoimmunology 7, e1380764, (2018)), adding new signaling binding motifs (Salter, A. I. et al., Sci Signal 14, (2021)), replacing the intracellular part with that from TCR (HIT) (Mansilla-Soto, J. et al., Nat Med 28, 345-352, (2022)), dual targeting by CAR together with chimeric costimulatory receptors (Katsarou, A. et al., Sci Transl Med 13, eabh1962, (2021)), implementing other signaling pathways (Wilkens, A. B. et al. Blood 140, 2261-2275, (2022)), and/or host cell gene knockdown or knockout (Mirzaei, et al., Cancer Lett., 2018 Jun. 1; 423:95-104. doi: 10.1016/j.canlet.2018.03.010. Epub 2018 Mar. 12; “BATF Knockout in CAR T Cells Prevents Exhaustion in Solid Tumors” Cancer Discov (2022) 12 (12): OF9, doi.org/10.1158/2159-8290.CD-RW2022-188, Kamali, et al., BMC Biotechnol 21, 9 (2021). doi.org/10.1186/st2896-020-00665-4, Dai, et al., Nat Methods., 2019 March; 16(3):247-254. doi: 10.1038/s41592-019-0329-7. Epub 2019 Feb. 25.) to increase activity, prevent exhaustion, reduce toxicity, or achieve more than additive effects.

### A. Methods of Treatment

Methods of treatment including cells and other therapeutic agents the provided polypeptides and fusions proteins such as CAR-mast are described. In preferred embodiments, the methods include Adoptive Cell Therapy (ACT) employing cells, e.g., mast cells, expressing recombinant CAR-mast fusion proteins. The CAR cells including CAR-mast fusion proteins can proinflammatory and anti-tumor activity. For example, the CAR cells including CAR-mast fusion proteins show enhanced cytotoxicity of a host mast cell towards target cells, increased expression one or more inflammatory cytokines and/or chemokines such as those discussed in the experiments below, recruitment or attraction T and/or NK cells, e.g., into tumor tissues, or a combination thereof.

An exemplary method involves treating a subject (e.g., a human) having a disease, disorder, or condition by administering to the subject an effective amount of a pharmaceutical composition including cells, e.g., genetically modified cells, including mast intracellular signaling domain fusion polypeptides such as CAR-mast fusion proteins. In some embodiments, the methods administer the modified cells, e.g., CAR-mast cells, to a subject (e.g., a human) having a disease, disorder, or condition in an amount effective to treat the disease, disorder, or condition. For example, in some embodiments, the methods treat a disease or disorder associated with an elevated expression or specific expression of an antigen by administering to the subject an effective amount of a pharmaceutical composition including cells modified to express recombinant CAR-mast fusion proteins. In some embodiments, the methods treat a subject having a disease, disorder, or condition associated with an elevated expression or specific expression of an antigen by administering to the subject an effective amount of a pharmaceutical composition including cells modified to contain a CAR-mast that targets the antigen.

Methods of using mast intracellular fusion proteins, such as a CAR-mast, to treat a disease or disorder are provided. Typically, the methods enhance ACT, for example, by providing CAR-mast-bearing cells with enhanced therapeutic efficacy in vivo. The CAR-mast cells have prolonged survival/serum residency time in vivo relative to CAR cells lacking the CAR-mast fusion protein or CAR lacking a mast intracellular signaling domain. Methods of treating a subject having a disease, disorder, or condition including administering to the subject an effective amount of a pharmaceutical composition including live, viable cells engineered to express a CAR-mast and/or another mast intracellular signaling domain fusion protein are provided. In some embodiments, when the methods treat a subject having a disease, disorder, or condition associated with an elevated expression or specific expression of an antigen, the methods include administering to the subject an effective amount of a cell, e.g., a mast cell, modified to express a CAR-mast that targets the antigen. For example, in some forms, the methods treat a subject having a disease, disorder, or condition by administering to the subject an effective amount of a pharmaceutical composition having a genetically modified cell, where the cell is modified by introducing to the cell:
(i) a vector, optionally including a transposon encoding a CAR-mast and/or other mast intracellular signaling domain fusion protein; and
(ii) causing the CAR-mast and/or other mast intracellular signaling domain-fusion protein to be expressed by the cell. The cell can have been isolated from the subject having the disease, disorder, or condition, or from a healthy donor, prior to genetic modification.

In additional or alternative to ACT, CAR-mast-expressing cells can be created in vivo in a subject in need thereof by introducing an effective amount of nucleic acids (e.g., RNA, viral vectors, etc.) encoding the CAR-mast into the subject. In some embodiments, the constructs are targeted to the tumor microenvironment using, e.g., targeted delivery vehicles. See, e.g., Xin, et al., Front. Oncol., 10 Feb. 2022, Sec. Cancer Immunity and Immunotherapy, Volume 12-20221 doi.org/10.3389/fonc.2022.809754.

### 1. Diseases to be Treated

Methods of treating diseases and/or disorders in a subject in need thereof are provided. The subject to be treated can have a disease, disorder, or condition such as but not limited to, cancer, an immune system disorder such autoimmune disease, an inflammatory disease, a neuronal disorder, HIV/AIDS, diabetes, a cardiovascular disease, an infectious disease, or combinations thereof. The disease, disorder, or condition can be associated with an elevated expression or specific expression of an antigen.

a. Cancer

In some embodiments, the methods treat or prevent cancer. In some forms, the methods treat or prevent cancer or other proliferative disease or disorder in a subject identified as having, or at risk of having cancer or other proliferative disease or disorder. Cancer is a disease of genetic instability, allowing a cancer cell to acquire the hallmarks proposed by Hanahan and Weinberg, including (i) self-sufficiency in growth signals; (ii) insensitivity to anti-growth signals; (iii) evading apoptosis; (iv) sustained angiogenesis; (v) tissue invasion and metastasis; (vi) limitless replicative potential; (vii) reprogramming of energy metabolism; and (viii) evading immune destruction (Cell., 144:646-674, (2011)).

Tumors, which can be treated in accordance with the disclosed methods, are classified according to the embryonic origin of the tissue from which the tumor is derived. Carcinomas are tumors arising from endodermal or ectodermal tissues such as skin or the epithelial lining of internal organs and glands. Sarcomas, which arise less frequently, are derived from mesodermal connective tissues such as bone, fat, and cartilage. The leukemias and lymphomas are malignant tumors of hematopoietic cells of the bone marrow. Leukemias proliferate as single cells, whereas lymphomas tend to grow as tumor masses. Malignant tumors may show up at numerous organs or tissues of the body to establish a cancer.

The disclosed compositions and methods of treatment thereof are generally suited for treatment of carcinomas, sarcomas, lymphomas and leukemias. The described compositions and methods are useful for treating, or alleviating subjects having benign or malignant tumors by delaying or inhibiting the growth/proliferation or viability of tumor cells in a subject, reducing the number, growth, or size of tumors, inhibiting, or reducing metastasis of the tumor, and/or inhibiting or reducing symptoms associated with tumor development or growth.

The types of cancer that can be treated with the provided compositions and methods include, but are not limited to, cancers such as vascular cancer such as multiple myeloma, adenocarcinomas, and sarcomas, of bone, bladder, brain, breast, cervical, colorectal, esophageal, kidney, liver, lung, nasopharyngeal, pancreatic, prostate, skin, stomach, and uterine. In some forms, the compositions are used to treat multiple cancer types concurrently. The compositions can also be used to treat metastases or tumors at multiple locations.

Exemplary tumor cells include, but are not limited to, tumor cells of cancers, including leukemias including, but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as, but not limited to, chronic myelocytic (granulocytic) leukemia, chronic lymphocytic leukemia, hairy cell leukemia; polycythemia vera; lymphomas such as, but not limited to, Hodgkin's disease, non-Hodgkin's disease; multiple myelomas such as, but not limited to, smoldering multiple myeloma, nonsecretory myeloma, osteosclerotic myeloma, plasma cell leukemia, solitary plasmacytoma and extramedullary plasmacytoma; Waldenström's macroglobulinemia; monoclonal gammopathy of undetermined significance; benign monoclonal gammopathy; heavy chain disease; bone and connective tissue sarcomas such as, but not limited to, bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors including, but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including, but not limited to, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer, including, but not limited to, pheochromocytoma and adrenocortical carcinoma; thyroid cancer such as but not limited to papillary or follicular thyroid cancer, medullary thyroid cancer and anaplastic thyroid cancer; pancreatic cancer, including, but not limited to, insulinoma, gastrinoma, glucagonoma, vipoma, somatostatin-secreting tumor, and carcinoid or islet cell tumor; pituitary cancers including, but not limited to, Cushing's disease, prolactin-secreting tumor, acromegaly, and diabetes insipius; eye cancers including, but not limited to, ocular melanoma such as iris melanoma, choroidal melanoma, and ciliary body melanoma, and retinoblastoma; vaginal cancers, including, but not limited to, squamous cell carcinoma, adenocarcinoma, and melanoma; vulvar cancer, including, but not limited to, squamous cell carcinoma, melanoma, adenocarcinoma, basal cell carcinoma, sarcoma, and Paget's disease; cervical cancers including, but not limited to, squamous cell carcinoma, and adenocarcinoma; uterine cancers including, but not limited to, endometrial carcinoma and uterine sarcoma; ovarian cancers including, but not limited to, ovarian epithelial carcinoma, borderline tumor, germ cell tumor, and stromal tumor; esophageal cancers including, but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers including, but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers including, but not limited to, hepatocellular carcinoma and hepatoblastoma, gallbladder cancers including, but not limited to, adenocarcinoma; cholangiocarcinoma including, but not limited to, papillary, nodular, and diffuse; lung cancers including, but not limited to, non-small cell lung cancer, squamous cell carcinoma (epidermoid carcinoma), adenocarcinoma, large-cell carcinoma and small-cell lung cancer; testicular cancers including, but not limited to, germinal tumor, seminoma, anaplastic, classic (typical), spermatocytic, nonseminoma, embryonal carcinoma, teratoma carcinoma, choriocarcinoma (yolk-sac tumor), prostate cancers including, but not limited to, adenocarcinoma, leiomyosarcoma, and rhabdomyosarcoma; penal cancers; oral cancers including, but not limited to, squamous cell carcinoma; basal cancers; salivary gland cancers including, but not limited to, adenocarcinoma, mucoepidermoid carcinoma, and adenoid cystic carcinoma; pharynx cancers including, but not limited to, squamous cell cancer, and verrucous; skin cancers including, but not limited to, basal cell carcinoma, squamous cell carcinoma and melanoma, superficial spreading melanoma, nodular melanoma, lentigo malignant melanoma, acral lentiginous melanoma; kidney cancers including, but not limited to, renal cell cancer, adenocarcinoma, hypernephroma, fibrosarcoma, transitional cell cancer (renal pelvis and/or uterer); Wilms' tumor; bladder cancers including, but not limited to, transitional cell carcinoma, squamous cell cancer, adenocarcinoma, and carcinosarcoma. For a review of such disorders, see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

b. Immune System Disorders

In some embodiments, the methods administer modified cells including CAR-mast and/or other mast intracellular signaling domain-fusion protein(s) to treat or prevent one or more immune system disorders, including autoimmune diseases.

Under certain circumstances, the ability of the immune system to distinguish self from foreign antigens can be misdirected against healthy tissues, resulting in the undesirable attack and destruction of normal host cells (i.e., autoimmune diseases). Autoimmune diseases include over 100 types of diseases, with varied etiology and prognoses based on factors such as the affected region, the age of onset, response to the therapeutic agents and clinical manifestation may vary among different people (Muhammad, et al., Chimeric Antigen Receptor Based Therapy as a Potential Approach in Autoimmune Diseases: How Close Are We to the Treatment, Frontiers in Immunology, 11 (2020)).

Auto-antibody-secreting B lymphocytes and self-reactive T-lymphocytes play a key role in the development of autoimmune diseases. Based on the extent of tissue damage, autoimmunity is classified into two general categories, including organ-specific and systemic autoimmune. The former involves a specific area of the body such as type I diabetes (T1D), multiple sclerosis (MS), rheumatoid arthritis (RA), inflammatory bowel diseases (IBDs), and myasthenia gravis (MG), while the latter affects multiple regions of the body, causing systemic lupus erythematosus (SLE) and Sjögren's syndrome (SS). Therefore, in some forms, the methods treat or prevent one or more organ-specific autoimmune diseases in a subject. In other forms, the methods treat or prevent one or more systemic autoimmune diseases in a subject.

In some forms, the methods reduce or prevent one or more physiological processes associated with the development or progression of autoimmune disease in a subject. For example, in some forms, the methods reduce or prevent one or more of epitope spreading, for example, where infections alter the primary epitope into the secondary epitope or form several neoepitopes on antigen-presenting cells; bystander activation or pre-primed autoreactive T cell activation in a T cell receptor (TCR)-independent manner; persistent virus infection, or the constant presence of viral antigens that prompt immune responses; or immunological cross-reactivity between a host and pathogen, for example, due to shared immunologic epitopes or sequence similarities. Non-limiting examples of immune system disorders that can be treated or prevented by the methods include 22q11.2 deletion syndrome, Achondroplasia and severe combined immunodeficiency, Adenosine Deaminase 2 deficiency, Adenosine deaminase deficiency, Adult-onset immunodeficiency with anti-interferon-gamma autoantibodies, Agammaglobulinemia, non-Bruton type, Aicardi-Goutieres syndrome, Aicardi-Goutieres syndrome type 5, Allergic bronchopulmonary aspergillosis, Alopecia, Alopecia totalis, Alopecia universalis, Amyloidosis AA, Amyloidosis familial visceral, Ataxia telangiectasia, Autoimmune lymphoproliferative syndrome, Autoimmune lymphoproliferative syndrome due to CTLA4 haploinsuffiency, Autoimmune polyglandular syndrome type 1, Autosomal dominant hyper IgE syndrome, Autosomal recessive early-onset inflammatory bowel disease, Autosomal recessive hyper IgE syndrome, Bare lymphocyte syndrome 2, Barth syndrome, Blau syndrome, Bloom syndrome, Bronchiolitis obliterans, C1q deficiency, Candidiasis familial chronic mucocutaneous, autosomal recessive, Cartilage-hair hypoplasia, CHARGE syndrome, Chediak-Higashi syndrome, Cherubism, Chronic atypical neutrophilic dermatosis with lipodystrophy and elevated temperature, Chronic graft versus host disease, Chronic granulomatous disease, Chronic Infantile Neurological Cutaneous Articular syndrome, Chronic mucocutaneous candidiasis (CMC), Cohen syndrome, Combined immunodeficiency with skin granulomas, Common variable immunodeficiency, Complement component 2 deficiency, Complement component 8 deficiency type 1, Complement component 8 deficiency type 2, Congenital pulmonary alveolar proteinosis, Cryoglobulinemia, Cutaneous mastocytoma, Cyclic neutropenia, Deficiency of interleukin-1 receptor antagonist, Dendritic cell, monocyte, B lymphocyte, and natural killer lymphocyte deficiency, Dyskeratosis congenital, Dyskeratosis congenita autosomal dominant, Dyskeratosis congenita autosomal recessive, Dyskeratosis congenita X-linked, Epidermodysplasia verruciformis, Familial amyloidosis, Finnish type, Familial cold autoinflammatory syndrome, Familial Mediterranean fever, Familial mixed cryoglobulinemia, Felty's syndrome, Glycogen storage disease type 1B, Griscelli syndrome type 2, Hashimoto encephalopathy, Hashimoto's syndrome, Hemophagocytic lymphohistiocytosis, Hennekam syndrome, Hepatic venoocclusive disease with immunodeficiency, Hereditary folate malabsorption, Hermansky Pudlak syndrome 2, Herpes simplex encephalitis, Hoyeraal Hreidarsson syndrome, Hyper IgE syndrome, Hyper-IgD syndrome, ICF syndrome, Idiopathic acute eosinophilic pneumonia, Idiopathic CD4 positive T-lymphocytopenia, IL12RB1 deficiency, Immune defect due to absence of thymus, Immune dysfunction with T-cell inactivation due to calcium entry defect 1, Immune dysfunction with T-cell inactivation due to calcium entry defect 2, Immunodeficiency with hyper IgM type 1, Immunodeficiency with hyper IgM type 2, Immunodeficiency with hyper IgM type 3, Immunodeficiency with hyper IgM type 4, Immunodeficiency with hyper IgM type 5, Immunodeficiency with thymoma, Immunodeficiency without anhidrotic ectodermal dysplasia, Immunodysregulation, polyendocrinopathy and enteropathy X-linked, Immunoglobulin A deficiency 2, Intestinal atresia multiple, IRAK-4 deficiency, Isolated growth hormone deficiency type 3, Kawasaki disease, Large granular lymphocyte leukemia, Leukocyte adhesion deficiency type 1, LRBA deficiency, Lupus, Lymphocytic hypophysitis, Majeed syndrome, Melkersson-Rosenthal syndrome, MHC class 1 deficiency, Muckle-Wells syndrome, Multifocal fibrosclerosis, Multiple sclerosis, MYD88 deficiency, Neonatal systemic lupus erythematosus, Netherton syndrome, Neutrophil-specific granule deficiency, Nijmegen breakage syndrome, Omenn syndrome, Osteopetrosis autosomal recessive 7, Palindromic rheumatism, Papillon Lefevre syndrome, Partial androgen insensitivity syndrome, PASLI disease, Pearson syndrome, Pediatric multiple sclerosis, Periodic fever, aphthous stomatitis, pharyngitis and adenitis, PGM3-CDG, Poikiloderma with neutropenia, Pruritic urticarial papules plaques of pregnancy, Purine nucleoside phosphorylase deficiency, Pyogenic arthritis, pyoderma gangrenosum and acne, Relapsing polychondritis, Reticular dysgenesis, Sarcoidosis, Say Barber Miller syndrome, Schimke immunoosseous dysplasia, Schnitzler syndrome, Selective IgA deficiency, Selective IgM deficiency, Severe combined immunodeficiency, Severe combined immunodeficiency due to complete RAG1/2 deficiency, Severe combined immunodeficiency with sensitivity to ionizing radiation, Severe combined immunodeficiency, Severe congenital neutropenia autosomal recessive 3, Severe congenital neutropenia X-linked, Shwachman-Diamond syndrome, Singleton-Merten syndrome, SLC35C1-CDG (CDG-IIc), Specific antibody deficiency, Spondyloenchondrodysplasia, Stevens-Johnson syndrome, T-cell immunodeficiency, congenital alopecia and nail dystrophy, TARP syndrome, Trichohepatoenteric syndrome, Tumor necrosis factor receptor-associated periodic syndrome, Twin to twin transfusion syndrome, Vici syndrome, WHIM syndrome, Wiskott Aldrich syndrome, Woods Black Norbury syndrome, X-linked agammaglobulinemia, X-linked lymphoproliferative syndrome, X-linked lymphoproliferative syndrome 1, X-linked lymphoproliferative syndrome 2, X-linked magnesium deficiency with Epstein-Barr virus infection and neoplasia, X-linked severe combined immunodeficiency, and ZAP-70 deficiency.

The disclosed compositions and methods can also be used to treat autoimmune diseases or disorders. Exemplary autoimmune diseases or disorders, which are not mutually exclusive with the immune system disorders described above, include Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticarial, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatic, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, Vogt-Koyanagi-Harada Disease, and Wegener's granulomatosis (or Granulomatosis with Polyangiitis (GPA)).

c. Other Disease or Disorders

In some forms the methods administer modified cells including CAR-mast and/or other mast intracellular signaling domain-fusion protein(s) to treat one or more additional disease or disorder in a subject in need thereof. For example, in some forms the methods treat one or more genetic disease or disorders in a subject, such as a hereditary genetic disease or disorder, or a somatic genetic disease or disorder in a subject.

Any of the methods can include treating a subject having an underlying disease or disorder. For example, in some forms, the methods treat a disease or disorder, such as a cancer or auto-immune disease in a patient having another disease or disorder, such as diabetes, a bacterial infection (e.g., Tuberculosis), viral infection (e.g., Hepatitis, HIV, HPV infection, etc.), or a drug-associated disease or disorder. In some forms, the methods treat an immunocompromised subject. In some forms, the methods treat a subject having a disease of the kidney, liver, heart, lung, brain, bladder, reproductive system, bowel/intestines, stomach, bones, or skin.

### B. Effective Amounts

In some forms the methods administer modified cells including CAR-mast and/or other mast intracellular signaling domain-fusion protein(s) in an effective amount. The effective amount or therapeutically effective amount of a pharmaceutical compositions including modified cells, such as therapeutic mast cells, can be a dosage sufficient to treat, inhibit, or alleviate one or more symptoms of a disease or disorder, such as a cancer or autoimmune disease, or to otherwise provide a desired pharmacologic and/or physiologic effect, for example, reducing, inhibiting, or reversing one or more of the underlying pathophysiological mechanisms underlying a disease or disorder, such as cancer or autoimmune disease.

In some forms, when administration of the pharmaceutical compositions including modified cells, such as therapeutic mast cells, including CAR-mast and/or other mast intracellular signaling domain-fusion protein(s) elicits an anti-cancer response, the amount administered can be expressed as the amount effective to achieve a desired anti-cancer effect in the recipient. For example, in some forms, the amount of the pharmaceutical compositions including modified cells, such as therapeutic cells, e.g., mast cells, is effective to inhibit the viability or proliferation of cancer cells in the recipient. In some forms, the amount of the pharmaceutical composition including modified cells, such as therapeutic mast cells, is effective to reduce the tumor burden in the recipient, or reduce the total number of cancer cells, and combinations thereof. In other forms, the amount of the pharmaceutical compositions including modified cells, such as therapeutic mast cells, is effective to reduce one or more symptoms or signs of cancer in a cancer patient, or signs of an autoimmune disease in a patient having an autoimmune disease or disorder. Signs of cancer can include cancer markers, such as PSMA levels in the blood of a patient.

The effective amount of the pharmaceutical compositions including modified cells, such as therapeutic mast cells, that is required will vary from subject to subject, depending on the species, age, weight and general condition of the subject, the severity of the disorder being treated, and its mode of administration. Thus, it is not possible to specify an exact amount for every pharmaceutical composition. However, an appropriate amount can be determined by one of ordinary skill in the art using only routine experimentation given the teachings herein. For example, effective dosages and schedules for administering the pharmaceutical compositions including therapeutic mast cells can be determined empirically, and making such determinations is within the skill in the art. In some forms, the dosage ranges for the administration of the compositions including therapeutic mast cells are those large enough to effect reduction in cancer cell proliferation or viability, or to reduce tumor burden for example.

The dosage should not be so large as to cause adverse side effects, such as unwanted cross-reactions, anaphylactic reactions, and the like. Generally, the dosage will vary with the age, condition, and sex of the patient, route of administration, whether other drugs are included in the regimen, and the type, stage, and location of the disease to be treated. The dosage can be adjusted by the individual physician in the event of any counter-indications. It will also be appreciated that the effective dosage of the composition including therapeutic mast cells used for treatment can increase or decrease over the course of a particular treatment. Changes in dosage can result and become apparent from the results of diagnostic assays.

Dosage can vary, and can be administered in one or more dose administrations daily, for one or several days. Guidance can be found in the literature for appropriate dosages for given classes of pharmaceutical products. Optimal dosing schedules can be calculated from measurements of drug accumulation in the body of the subject or patient. Persons of ordinary skill can easily determine optimum dosages, dosing methodologies and repetition rates. Optimum dosages can vary depending on the relative potency of individual pharmaceutical compositions and can generally be estimated based on EC₅₀s found to be effective in in vitro and in vivo animal models.

It can generally be stated that a pharmaceutical composition containing CAR-mast cells described herein can be administered at a dosage of 10⁴ to 10⁹ cells/kg body weight, preferably 10⁵ to 10⁷ cells/kg body weight, including all integer values within those ranges. In some forms, patients can be treated by infusing a disclosed pharmaceutical composition containing CAR-mast construct expressing cells (e.g., mast cells) in the range of about 10⁴ to 10¹² or more cells per square meter of body surface (cells/m).

The infusion can be repeated as often and as many times as the patient can tolerate until the desired response is achieved. CAR-mast cell compositions can also be administered once or multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly. In some forms, the unit dosage is in a unit dosage form for intravenous injection. In some forms, the unit dosage is in a unit dosage form for oral administration. In some forms, the unit dosage is in a unit dosage form for inhalation. In some forms, the unit dosage is in a unit dosage form for intra-tumoral injection.

Treatment can be continued for an amount of time sufficient to achieve one or more desired therapeutic goals, for example, a reduction of the amount of cancer cells relative to the start of treatment, or complete absence of cancer cells in the recipient. Treatment can be continued for a desired period of time, and the progression of treatment can be monitored using any means known for monitoring the progression of anti-cancer treatment in a patient. In some forms, administration is carried out every day of treatment, or every week, or every fraction of a week. In some forms, treatment regimens are carried out over the course of up to two, three, four or five days, weeks, or months, or for up to 6 months, or for more than 6 months, for example, up to one year, two years, three years, or up to five years.

The efficacy of administration of a particular dose of the pharmaceutical compositions including modified cells, such as therapeutic mast cells, according to the methods described herein can be determined by evaluating the aspects of the medical history, signs, symptoms, and objective laboratory tests that are known to be useful in evaluating the status of a subject in need for the treatment of cancer or other diseases and/or conditions. These signs, symptoms, and objective laboratory tests will vary, depending upon the particular disease or condition being treated or prevented, as will be known to any clinician who treats such patients or a researcher conducting experimentation in this field. For example, if, based on a comparison with an appropriate control group and/or knowledge of the normal progression of the disease in the general population or the particular individual: (1) a subject's physical condition is shown to be improved (e.g., a tumor has partially or fully regressed), (2) the progression of the disease or condition is shown to be stabilized, or slowed, or reversed, or (3) the need for other medications for treating the disease or condition is lessened or obviated, then a particular treatment regimen will be considered efficacious. In some forms, efficacy is assessed as a measure of the reduction in tumor volume and/or tumor mass at a specific time point (e.g., 1-5 days, weeks, or months) following treatment.

### C. Modes of Administration

In some embodiments the methods administer modified cells including CAR-mast and/or other mast intracellular signaling domain-fusion protein(s) in combination with a pharmaceutically acceptable carrier. The compositions described herein can be conveniently formulated into pharmaceutical compositions composed of one or more of the compounds in association with a pharmaceutically acceptable carrier. See, e.g., Remington's Pharmaceutical Sciences, latest edition, by E.W. Martin Mack Pub. Co., Easton, PA, which discloses typical carriers and conventional methods of preparing pharmaceutical compositions that can be used in conjunction with the preparation of formulations of the therapeutics described herein and which is incorporated by reference herein. These most typically would be standard carriers for administration of compositions to humans. In one aspect, for humans and non-humans, these include solutions such as sterile water, saline, and buffered solutions at physiological pH. Other therapeutics can be administered according to standard procedures used by those skilled in the art.

The pharmaceutical compositions including modified cells, such as therapeutic mast cells, described herein can include, but are not limited to, carriers, thickeners, diluents, buffers, preservatives, surface active agents and the like in addition to the therapeutic(s) of choice.

Pharmaceutical compositions containing one or more modified cells, such as therapeutic mast cells including CAR-mast and/or other mast intracellular signaling domain-fusion protein(s), and optionally one or more additional therapeutic agents can be administered to the subject in a number of ways depending on whether local or systemic treatment is desired, and on the area to be treated. Thus, for example, a pharmaceutical composition including modified cells, such as therapeutic mast cells, can be administered as an intravenous infusion, or directly injected into a specific site, for example, into or surrounding a tumor. Moreover, a pharmaceutical composition can be administered to a subject as an ophthalmic solution and/or ointment to the surface of the eye, vaginally, rectally, intranasally, orally, by inhalation, or parenterally, for example, by intradermal, subcutaneous, intramuscular, intraperitoneal, intrarectal, intraarterial, intralymphatic, intravenous, intrathecal and intratracheal routes. In some forms, the compositions are administered directly into a tumor or tissue, e.g., stereotactically.

Parenteral administration, if used, is generally characterized by injection. Injectables can be prepared in conventional forms, either as liquid solutions or suspensions, solid forms suitable for solution or suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein. Suitable parenteral administration routes include intravascular administration (e.g., intravenous bolus injection, intravenous infusion, intra-arterial bolus injection, intra-arterial infusion and catheter instillation into the vasculature); peri- and intra-tissue injection (e.g., intraocular injection, intra-retinal injection, or sub-retinal injection); subcutaneous injection or deposition including subcutaneous infusion (such as by osmotic pumps); direct application by a catheter or other placement device (e.g., an implant including a porous, non-porous, or gelatinous material).

Preparations for parenteral administration include sterile aqueous or non-aqueous solutions, suspensions, and emulsions which can also contain buffers, diluents, and other suitable additives. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions, or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, antioxidants, chelating agents, and inert gases and the like.

Administration of the pharmaceutical compositions containing one or more genetically modified cells (e.g., CAR-mast cells) can be localized (i.e., to a particular region, physiological system, tissue, organ, or cell type) or systemic.

It is to be understood that the disclosed method and compositions are not limited to specific synthetic methods, specific analytical techniques, or to particular reagents unless otherwise specified, and, as such, can vary. It is also to be understood that the terminology used herein is for the purpose of describing particular embodiments only and is not intended to be limiting.

### D. Combination Therapy

In some embodiments the methods administer modified cells including CAR-mast and/or other mast intracellular signaling domain-fusion protein(s) in combination with other therapeutic agents or treatment modalities. Any of the disclosed pharmaceutical compositions including modified cells, such as therapeutic mast cells (e.g., containing a population of CAR-mast cells), can be used alone, or in combination with other therapeutic agents or treatment modalities, for example, chemotherapy or stem-cell transplantation. As used herein, “combination” or “combined” refer to either concomitant, simultaneous, or sequential administration of the therapeutics.

In some forms, the pharmaceutical compositions and other therapeutic agents are administered separately through the same route of administration. In other forms, the pharmaceutical compositions and other therapeutic agents are administered separately through different routes of administration. The combinations can be administered either concomitantly (e.g., as an admixture), separately but simultaneously (e.g., via separate intravenous lines into the same subject; one agent is given orally while the other agent is given by infusion or injection, etc.,), or sequentially (e.g., one agent is given first followed by the second).

Examples of preferred additional therapeutic agents include other conventional therapies known in the art for treating the desired disease, disorder, or condition. In some forms, the therapeutic agent is one or more other targeted therapies (e.g., a targeted cancer therapy) and/or immune-checkpoint blockage agents (e.g., anti-CTLA-4, anti-PD1, and/or anti-PDL1 agents such as antibodies).

The CAR-mast cells can also be used in combination with other forms of adaptive cell therapy including, but not limited to, CAR T cell therapy, CAR NK cell therapy, CAR macrophage therapy, e.g., against the same of a different antigen.

The compositions and methods described herein may be used as a first therapy, second therapy, third therapy, or combination therapy with other types of therapies known in the art, such as chemotherapy, surgery, radiation, gene therapy, immunotherapy, bone marrow transplantation, stem cell transplantation, targeted therapy, cryotherapy, ultrasound therapy, photodynamic therapy, radio-frequency ablation or the like, in an adjuvant setting or a neoadjuvant setting.

The disclosed pharmaceutical compositions and/or other therapeutic agents, procedures or modalities can be administered during periods of active disease, or during a period of remission or less active disease. The pharmaceutical compositions can be administered before the additional treatment, concurrently with the treatment, post-treatment, or during remission of the disease or disorder. When administered in combination, the disclosed pharmaceutical compositions, and the additional therapeutic agents (e.g., second or third agent), or all, can be administered in an amount or dose that is higher, lower or the same than the amount or dosage of each agent used individually, e.g., as a monotherapy. In certain forms, the administered amount or dosage of the disclosed pharmaceutical composition, the additional therapeutic agent (e.g., second or third agent), or all, is lower (e.g., at least 20%, at least 30%, at least 40%, or at least 50%) than the amount or dosage of each agent used individually, e.g., as a monotherapy (e.g., required to achieve the same therapeutic effect).

### 1. Additional Anti-Cancer Agents

In some embodiments, the methods administer one or more additional anti-cancer agents to a subject.

In the context of cancer, targeted therapies are therapeutic agents that block the growth and spread of cancer by interfering with specific molecules (“molecular targets”) that are involved in the growth, progression, and spread of cancer. Many different targeted therapies have been approved for use in cancer treatment. These therapies include hormone therapies, signal transduction inhibitors, gene expression modulators, apoptosis inducers, angiogenesis inhibitors, immunotherapies, and toxin delivery molecules. Numerous antineoplastic drugs can be used in combination with the disclosed pharmaceutical compositions. In some forms, the additional therapeutic agent is a chemotherapeutic or antineoplastic drug. The majority of chemotherapeutic drugs can be divided into alkylating agents, antimetabolites, anthracyclines, plant alkaloids, topoisomerase inhibitors, monoclonal antibodies, and other anti-tumor agents.

### 2. Additional Therapeutic Agents Against Autoimmune Diseases

In some embodiments, the methods also include administering one or more conventional therapies for autoimmune diseases to the subject.

Exemplary therapies for autoimmune diseases include immunosuppressive agents, such as steroids or cytostatic drugs, analgesics, non-steroidal anti-inflammatory drugs, glucocorticoids, immunosuppressive and immunomodulatory agents, such as methotrexate, leflunomide, hydroxychloroquine, and sulfasalazine. In some forms, the methods administer one or more disease-modifying antirheumatic drugs (DMARDs). In some forms, the methods administer one or more biologic agents for localized treatment (i.e., agents that do not affect the entire immune system), such as TNF-α inhibitors, belimumab and rituximab depleting B cells, T-cell co-stimulation blocker, anti-interleukin 6 (IL-6), anti-IL-1, and protein kinase inhibitors. In other forms, the methods also administer one or more monoclonal antibodies (mAbs), such as anti-TNFα, anti-CD19, anti-CD20, anti-CD22, and anti-IL6R, or other mAbs that target multiple B cell subtypes, and other aberrant cells in autoimmune diseases.

### IV. Kits

The compositions, reagents, and other materials for cellular genomic engineering can be packaged together in any suitable combination as a kit useful for performing, or aiding in the performance of, the methods. It is useful if the components in a given kit are designed and adapted for use together in the method. For example, kits with one or more compositions for administration to a subject, may include a pre-measured dosage of the composition in a sterile needle, ampule, tube, container, or other suitable vessel. The kits may include instructions for dosages and dosing regimens.

Provided are kits containing CAR-mast and/or other mast intracellular signaling domain-fusion protein(s) within a vector (e.g., a viral vector) and/or mRNA encoding the the same, and instructional material for use thereof. In preferred forms, the kit includes a plurality of vectors, where each vector independently contains CAR-mast and/or other mast intracellular signaling domain-fusion protein(s) for insertion into a host cell genome, such as a CAR expression cassette. In some forms, the kit contains a population of cells (e.g., mast cells) optionally collectively containing the CAR-mast and/or other mast intracellular signaling domain-fusion protein(s). The instructional material can include a publication, a recording, a diagram, or any other medium of expression which can be used to communicate the usefulness of the compositions and methods of the kit. For example, the instructional material may provide instructions for methods using the kit components, such as performing transfections, transductions, infections, and conducting screens. In some forms, kits include a transposon that includes a promoter and/or polyadenylation signal operationally linked to a reporter gene and/or a CAR; in some forms, the kit includes a transposon including a CAR that is specific for an antigen selected from a cancer antigen, an inflammatory disease antigen, a neuronal disorder antigen, HIV/AIDS, a diabetes antigen, a cardiovascular disease antigen, an infectious disease antigen (including a viral antigen, a protozoan antigen, a bacterial antigen, and an allergen), an autoimmune disease antigen and an autoimmune disease antigen, or combinations thereof;

In exemplary embodiments, the kits include a nucleic acid and/or a vector expressing or encoding the CAR-mast and/or other mast intracellular signaling domain-fusion protein(s) and/or cells. Exemplary cells include a mast cell or mast progenitor cell, hematopoietic stem cell (HSC), etc.

The present invention can be further understood by the following numbered paragraphs:
1. A fusion polypeptide including
(a) a mast cell intracellular signaling domain; and
(b) an amino acid sequence that is heterologous to the mast cell intracellular signaling domain.
2. The polypeptide of paragraph 1, wherein the amino acid sequence of (a) includes the intracellular domain of IgE receptor (FcRI), a cytokine receptor (c-KIT), a G-protein-coupled receptor, or a toll-like receptor expressed in mast cells, or functional fragment or variant thereof.
3. The polypeptide of paragraphs 1 or 2 including an ITAM sequence.
4. The polypeptide of paragraph 3, wherein the ITAM sequence includes DGVYTGLSTRNQETYETLKHE (SEQ ID NO:11) or a functional variant thereof or a variant thereof having an amino acid sequence at least 70%, 75%, 80%, 85%, 90%, 95%, or 99% identical to SEQ ID NO:11.
5. The polypeptide of any one of paragraphs 1-4, wherein the amino acid sequence of (a) includes the amino acid sequence of any one of SEQ ID NOS:11-21, or a functional fragment thereof, or a variant thereof having an amino acid sequence at least 70%, 75%, 80%, 85%, 90%, 95%, or 99% identical to any one of SEQ ID NOS:11-21.
6. The polypeptide of any one of paragraphs 1-5, wherein (b) includes one or more of an extracellular domain, transmembrane domain, and/or a further intracellular domain.
7. The polypeptide of paragraph 6, wherein the polypeptide is a chimeric antigen receptor (CAR).
8. The polypeptide of paragraph 7, wherein the CAR includes an intracellular domain including (a), a transmembrane domain, and an extracellular domain.
9. The polypeptide of any one of paragraphs 6-8, wherein (b) includes a transmembrane domain and optionally stalk of CD8α, optionally of SEQ ID NO:4 or fragment or variant thereof with at least 70% sequence identity thereto, or CD28, optionally SEQ ID NO:10 or fragment or variant thereof with at least 70% sequence identity thereto.
10. The polypeptide of any one of paragraphs 1-9, wherein the CAR is specific for an antigen selected from the group consisting of a cancer antigen, an inflammatory disease antigen, a neuronal disorder antigen, HIV/AIDS, a diabetes antigen, a cardiovascular disease antigen, an infectious disease antigen (including a viral antigen, a protozoan antigen, a bacterial antigen, and an allergen), an autoimmune disease antigen and an autoimmune disease antigen, or combinations thereof.
11. The polypeptide of paragraph 10, wherein the CAR targets one or more antigens selected from the group consisting of B7H3, HER2, CD19, GD2, AFP, AKAP 4, ALK, Androgen receptor, B7H3, BCMA, Bcr Abl, BORIS, Carbonic, CD123, CD138, CD174, CD20, CD22, CD30, CD33, CD38, CD80, CD86, CEA, CEACAM5, CEACAM6, Cyclin, CYP1B1, EBV, EGFR, EGFR806, EGFRvIII, EpCAM, EphA2, ERG, ETV6 AML, FAP, Fos related antigen1, Fucosyl, fusion, GD3, GloboH, GM3, gp100, GPC3, HER 2/neu, HMWMAA, HPV E6/E7, hTERT, Idiotype, IL12, IL13RA2, IM19, IX, LCK, Legumain, IgK, LMP2, MAD CT 1, MAD CT 2, MAGE, MelanA/MART1, Mesothelin, MET, ML IAP, MUC1, Mutant p53, MYCN, NA17, NKG2D L, NY BR 1, NY ESO 1, NY ESO 1, OY TES1, p53, Page4, PAP, PAX3, PAX5, PD L1, PDGFR β, PLAC1, Polysialic acid, Proteinase3 (PR1), PSA, PSCA, PSMA, Ras mutant, RGS5, RhoC, ROR1, SART3, sLe(a), Sperm protein 17, SSX2, STn, Survivin, Tie2, Tn, TRP 2, Tyrosinase, VEGFR2, WT1, XAGE, Claudin-6, Claudin-18.2 and CD70.
12. The polypeptide of paragraph 11, wherein the antigen is a cancer antigen selected from the group consisting of B7H3, HER2, CD19, GD2, 41BB, 5T4, adenocarcinoma antigen, alpha fetoprotein, BAFF, B lymphoma cell, C242 antigen, CA 125, carbonic anhydrase 9 (CA IX), C MET, CCR4, CD152, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNT0888, CTLA 4, DR5, EGFR, EpCAM, CD3, FAP, fibronectin extra domain B, folate receptor 1, GD3 ganglioside, glycoprotein 75, GPNMB, HER2/neu, HGF, human scatter factor receptor kinase, IGF 1 receptor, IGF I, IgG1, L1 CAM, IL 13, IL 6, insulin-like growth factor I receptor, integrin α5β1, integrin αvβ3, MORAb 009, MS4A1, MUC1, mucin CanAg, N glycolylneuraminic acid, NPC 1C, PDGF R a, PDL192, phosphatidylserine, prostatic carcinoma cells, RANKL, RON, ROR1, SCH 900105, SDC1, SLAMF7, TAG 72, tenascin C, TGF beta 2, TGF β, TRAIL R1, TRAIL R2, tumor antigen CTAA16.88, VEGF A, VEGFR 1, VEGFR2, and vimentin.
13. The polypeptide of paragraph 12, wherein the CAR including an antigen binding domain, optionally wherein the antigen-binding domain is anti-B7H3, anti-HER2, anti-CD19, or anti-GD2, optionally of SEQ ID NOS:9, 34, 2, or 8, respectfully, or a fragment or variant thereof including the complementary determining regions (CDRs) thereof.
14. A nucleic acid including a nucleic acid encoding the polypeptide of any one of paragraphs 1-13.
15. The nucleic acid of paragraph 14, wherein the nucleic acid is RNA or DNA.
16. The nucleic acid of paragraph 14 or 15, wherein the nucleic acid is mRNA.
17. The nucleic acid of any one of paragraphs 14-16, wherein the nucleic acid includes an expression control sequence(s).
18. The nucleic acid of any one of paragraphs 14-17, wherein the nucleic acid is, or is encoded by a vector or a transposon.
19. The nucleic acid of paragraph 18, wherein the vector is a viral vector.
20. The nucleic acid of paragraph 19, wherein the viral vector is selected from the group consisting of a lentiviral vector, an Adeno-associated virus (AAV) vector, or an adenovirus vector, or a Herpes Simplex virus (HSV) vector, or a vesicular stomatitis (VSV) vector, or a human Bocavirus vector (hBoV), or a chimeric vector including a combination of any two or more of an Adeno-associated virus (AAV) vector, Herpes Simplex virus (HSV) vector, vesicular stomatitis (VSV) vector, or a human Bocavirus vector (hBoV).
21. The nucleic acid of paragraph 20, wherein the vector is a nucleic acid expression vector selected from the group consisting of a plasmid, a cosmid, and a replicon.
22. The nucleic acid of any one of paragraphs 14-21, wherein the nucleic acid includes a promotor.
23. An isolated cell including the polypeptide of any one of paragraphs 1-13, or the nucleic acid of any one of paragraphs 14-22.
24. The isolated cell of paragraph 23, wherein the cell is a mast cell, mast progenitor cell, or hematopoietic stem cell (HSC).
25. The isolated cell of paragraph 24, wherein the cell is a mast cell.
26. The isolated cell of paragraphs 23 or 24, wherein the cell is isolated from a subject in need of adoptive cell therapy.
27. The isolated cell of any one of paragraphs 23-26, wherein the polypeptide is a CAR.
28. The isolated cell of any one of paragraphs 23-26, wherein the intracellular domain of the CAR includes the amino acid sequence of any one of SEQ ID NOS: 11-21 or a fragment or variant thereof with at least 70% sequence identity thereto.
29. A population of cells derived by expanding the cell of any one of paragraphs 23-28.
30. A pharmaceutical composition including the population of cells of paragraph 29 and a pharmaceutically acceptable buffer, carrier, diluent, or excipient.
31. A method of treating a subject having a disease, disorder, or condition including administering to the subject an effective amount of the pharmaceutical composition of paragraph 30.
32. A method of treating a subject having a disease, disorder, or condition associated with an elevated expression or specific expression of an antigen, the method including administering to the subject an effective amount of a cell of any one of paragraphs 25-28, wherein the CAR targets the antigen.
33. The method of any one of paragraphs 31-32, wherein the cell is isolated from the subject having the disease, disorder, or condition prior to the introduction to the cell.
34. The method of any one of paragraphs 31-32, wherein the cell is isolated from a healthy donor.
35. The method of any one of paragraphs 31-34, wherein the cell is a mast cell.
36. The method of any one of paragraphs 33-35, wherein the mast cell is isolated as a bone marrow mast cell, a spleen mast cell, or as a mast progenitor cell or hematopoietic stem cell and differentiated ex vivo into a mast cell.
37. The method of any one of paragraphs 31-36, wherein the subject is a human.
38. The method of any one of paragraphs 31-37, wherein the subject has cancer.
39. The method of paragraph 38, wherein the cancer includes a solid tumor.
40. A composition or method as disclosed herein including in the text and drawings.

The present invention will be further understood by reference to the following non-limiting examples.

### EXAMPLES

### Example 1: Generation and Characterization of Mast Cell CARS Targeting CD19

### Materials and Methods

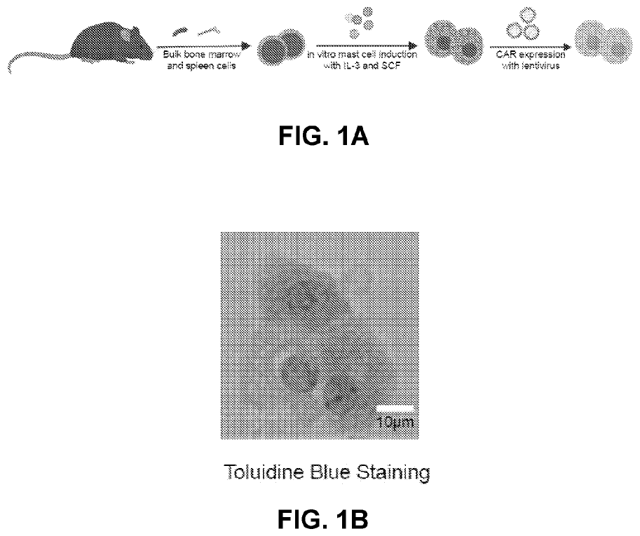

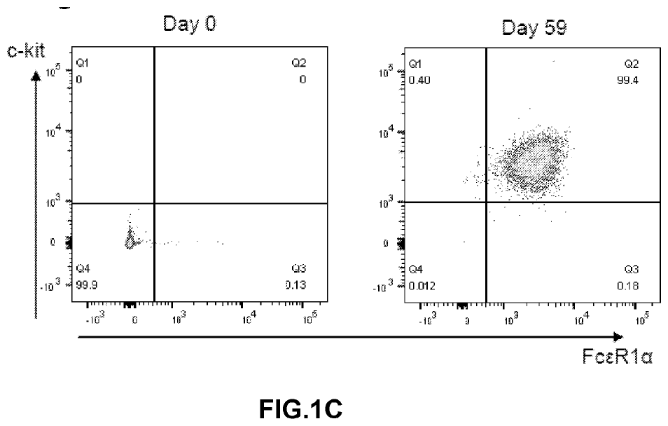

Mature mast cells were generated according to standard protocols. Spleen and Bone marrow from C57BL/6 mice were dissected and cultured in vitro in the presence of mouse SCF and IL-3 (FIG. 1A). Matured mast cells were obtained over 6-8 weeks, as indicated by the presence of intracellular granules (FIG. 1B) and the expression of mast cell marker c-kit and FcRI (FIG. 1C). These mast cells were further transduced with lentivirus encoding CARs as described below.

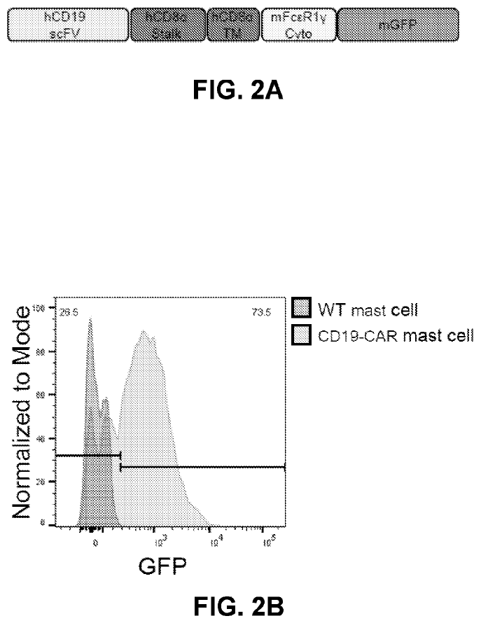

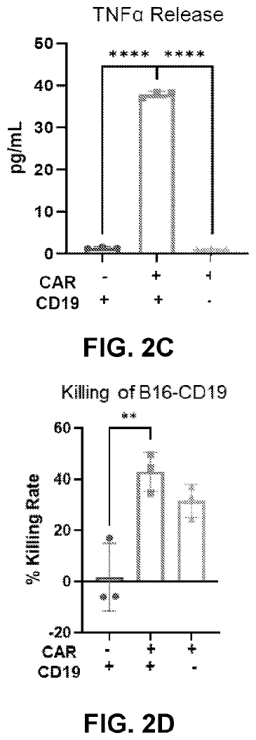

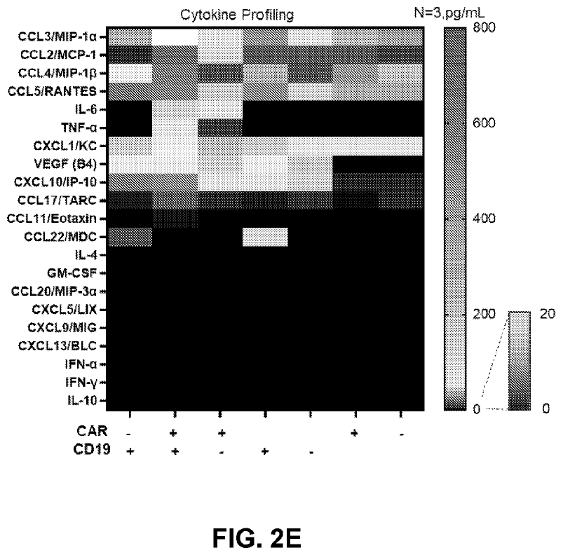

A mast cell CAR was constructed containing the immunoreceptor tyrosine-based activation motif (ITAM−) containing gamma chain of FcεRI, the stalk and transmembrane domain from CD8α, and the single-chain variable fragment (FMC63) targeting CD19, a model B cell cancer-associated antigen for testing CAR function (FIG. 2A). A monomer GFP is added at the C terminus of the structure for CAR labeling in later experiments. A lentivirus encoding this CD19 CAR was also generated. The sequences for primary elements of the CD19 CAR are provided below:

a.
Signal Peptide (SP):
(SEQ ID NO: 1)
MALPVTALLLPLALLLHAARP
b.
anti-Human CD19 single-chain variable fragment
(scFv):
(SEQ ID NO: 2)
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEITGGGGSGGGGSGGGGSEVKLQESGPGLVAPSQSLSVTCTVSGVS
LPDYGVSWIRQPPRKGLEWLGVIWGSETTYYNSALKSRLTIIKDNSKSQV
FLKMNSLQTDDTAIYYCAKHYYYGGSYAMDYWGQGTSVTVSS
c.
Human CD8α Stalk:
(SEQ ID NO: 3)
TTTPAPRPPTPAPTIASQPLSLRPEACRPAAGGAVHTRGLDFACD
d.
Human CD8α transmembrane domain:
(SEQ ID NO: 4)
IYIWAPLAGTCGVLLLSLVITLYCR
e.
Mouse FcεR1 gamma intracellular domain
(SEQ ID NO: 5)
RLKIQVRKAAIASREKADAVYTGLNTRSQETYETLKHEKPPQGSGS
f.
mGFP:
(SEQ ID NO: 6)
MVSKGEELFTGVVPILVELDGDVNGHKFSVSGEGEGDATYGKLTLKFICT
TGKLPVPWPTLVTTLTYGVQCFSRYPDHMKQHDFFKSAMPEGYVQERTIF
FKDDGNYKTRAEVKFEGDTLVNRIELKGIDFKEDGNILGHKLEYNYNSHN
VYIMADKQKNGIKVNFKIRHNIEDGSVQLADHYQQNTPIGDGPVLLPDNH
YLSTQSKLSKDPNEKRDHMVLLEFVTAAGITLGMDELYK.
g.
full CD19 CAR-mast sequence
(SEQ ID NO: 22)
MALPVTALLLPLALLLHAARPDIQMTQTTSSLSASLGDRVTISCRASQDI
SKYLNWYQQKPDGTVKLLIYHTSRLHSGVPSRFSGSGSGTDYSLTISNLE
QEDIATYFCQQGNTLPYTFGGGTKLEITGGGGSGGGGSGGGGSEVKLQES
GPGLVAPSQSLSVTCTVSGVSLPDYGVSWIRQPPRKGLEWLGVIWGSETT
YYNSALKSRLTIIKDNSKSQVFLKMNSLQTDDTAIYYCAKHYYYGGSYAM
DYWGQGTSVTVSSTTTPAPRPPTPAPTIASQPLSLRPEACRPAAGGAVHT
RGLDFACDIYIWAPLAGTCGVLLLSLVITLYCRRLKIQVRKAAIASREKA
DAVYTGLNTRSQETYETLKHEKPPQGSGSMVSKGEELFTGVVPILVELDG
DVNGHKFSVSGEGEGDATYGKLTLKFICTTGKLPVPWPTLVTTLTYGVQC
FSRYPDHMKQHDFFKSAMPEGYVQERTIFFKDDGNYKTRAEVKFEGDTLV
NRIELKGIDFKEDGNILGHKLEYNYNSHNVYIMADKQKNGIKVNFKIRHN
IEDGSVQLADHYQQNTPIGDGPVLLPDNHYLSTQSKLSKDPNEKRDHMVL
LEFVTAAGITLGMDELYK

### Results

Mast cells are activated by receptors different than those for T cells. A goal was to identify signaling domains that can be used for building a CAR to activate mast cells. The native receptors that activate mast cells include the high affinity IgE receptor (FcεRI), cytokine receptors (c-KIT), G-protein-coupled receptors, and toll-like receptors (Gilfillan, and Tkaczyk, Nat Rev Immunol 6:218-230 (2006)). Among those, FcεRI is of particular interest because 1) the pathway by which FcεRI triggers degranulation is well characterized and 2) FcεRI contains the immunoreceptor tyrosine-based activation motif (ITAM). ITAM is important for the signal transduction in CAR-T cells.

Thus, a mast cell CAR was constructed which included the ITAM-containing gamma chain of FcεRI, the stalk and transmembrane domain from CD8α, and the single-chain variable fragment (FMC63) targeting CD19, a model antigen for testing CAR function (FIG. 2A). A lentiviral construct encoding this CD19 CAR was generated. Lentivirus was produced from this construct and was found to infect mast cells with a high infection efficiency (FIG. 2B). These CAR-mast cells were robustly activated by a mouse melanoma cell line B16 that ectopically expressed CD19, as indicated by the secretion of TNFα detected by ELISA. No TNFα secretion was detected when plain mast cells were incubated with CD19+B16 cells or when CAR-mast cells were incubated with plain B16 cells (FIG. 2C), indicating that the observed activation is highly specific and depends on the presence of both CD19 and CAR. Furthermore, CAR-mast cells displayed cytotoxicity towards CD19+B16 cells whereas the cytotoxicity was attenuated when CD19 or CAR is absent (FIG. 2D). To determine what cytokines CAR-mast cells secret, a panel of common chemokines and cytokines were screened, and it was found that CAR-mast cells specifically secreted CCL2/3/4, TNFα and IL-6, when activated by CD19 (FIG. 2E).

### Example 2: Generation and Characterization of Mast Cell CARS Targeting GD2

### Materials and Methods

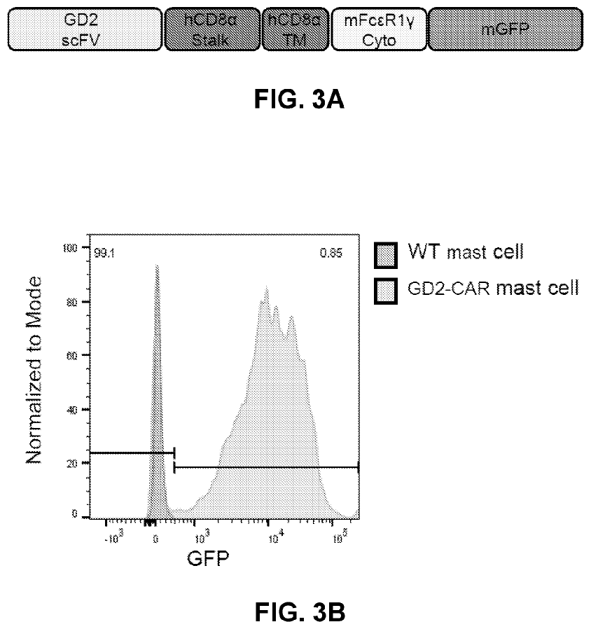

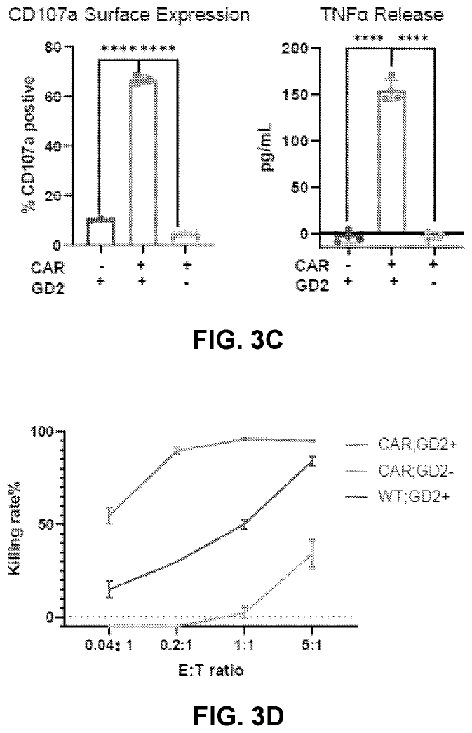

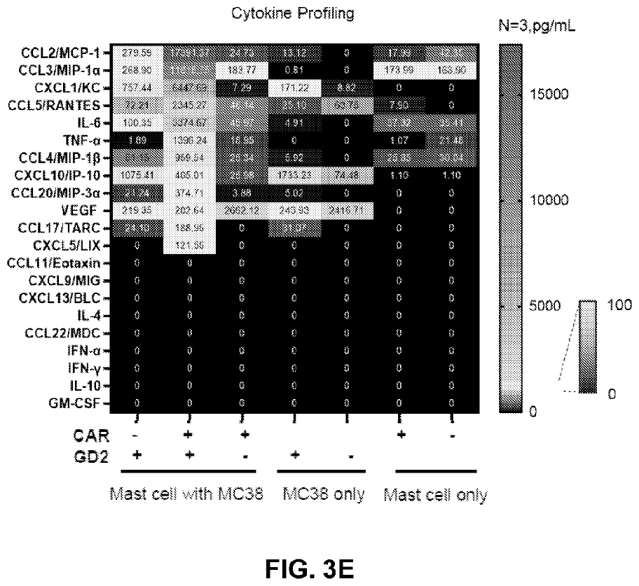

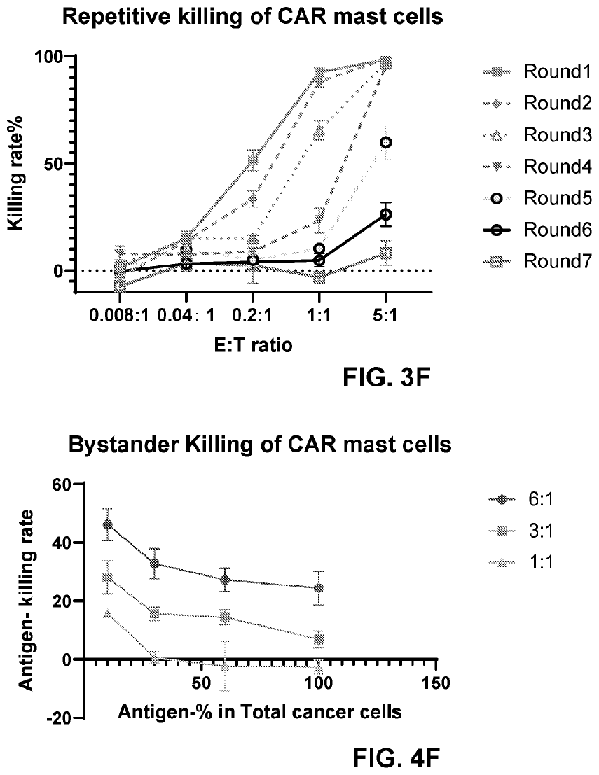

Mature mast cells were generated as described in Example 1 and illustrated in FIGS. 1A-1C. A CAR targeting GD2 (disialoganglioside) was generated (FIG. 3A). This GD2 CAR was introduced into mast cells by lentiviral transduction (FIG. 3B). These CAR-mast cells were co-cultured with GD2+B16 cells. To test the cytotoxicity of the GD2 CAR-mast cells, CAR-mast cells were co-cultured with GD2+ or GD2− B16 cells, using plain mast cells as a control.

The sequences for primary elements of the GD2 CAR are provided below:

a.
SP (Signal Peptides):
(SEQ ID NO: 1)
MALPVTALLLPLALLLHAARP
b.
HA tag:
(SEQ ID NO: 7)
YPYDVPDYAYPYDVPDYAWP
c.
anti-GD2 scFv:
(SEQ ID NO: 8)
HPAFLLIPQVQLVESGPGVVQPGRSLRISCAVSGFSVTNYGVHWVRQPPG
KGLEWLGVIWAGGITNYNSAFMSRLTISKDNSKNTVYLQMNSLRAEDTAM
YYCASRGGHYGYALDYWGQGTLVTVSSGSTSGSGKPGSSEGSTKGEIVMT
QTPATLSVSAGERVTITCKASQSVSNDVTWYQQKPGQAPRLLIYSASNRY
SGVPARFSGSGYGTEFTFTISSVQSEDFAVYFCQQDYSSFGQGTKLEIKT
S
d.
Human CD8α Stalk:
(SEQ ID NO: 3)
TTTPAPRPPTPAPTIASQPLSLRPEACRPAAGGAVHTRGLDFACD
e.
Human CD8α transmembrane domain:
(SEQ ID NO: 4)
IYIWAPLAGTCGVLLLSLVITLYCR
f.
Mouse FcεR1 gamma intracellular domain:
(SEQ ID NO: 5)
RLKIQVRKAAIASREKADAVYTGLNTRSQETYETLKHEKPPQGSGS
g.
mGFP
(SEQ ID NO: 6)
MVSKGEELFTGVVPILVELDGDVNGHKFSVSGEGEGDATYGKLTLKFICT
TGKLPVPWPTLVTTLTYGVQCFSRYPDHMKQHDFFKSAMPEGYVQERTIF
FKDDGNYKTRAEVKFEGDTLVNRIELKGIDFKEDGNILGHKLEYNYNSHN
VYIMADKQKNGIKVNFKIRHNIEDGSVQLADHYQQNTPIGDGPVLLPDNH
YLSTQSKLSKDPNEKRDHMVLLEFVTAAGITLGMDELYK.
h.
full GD2 CAR-mast sequence
(SEQ ID NO: 23)
MALPVTALLLPLALLLHAARPYPYDVPDYAYPYDVPDYAWPHPAFLLIPQ
VQLVESGPGVVQPGRSLRISCAVSGFSVTNYGVHWVRQPPGKGLEWLGVI
WAGGITNYNSAFMSRLTISKDNSKNTVYLQMNSLRAEDTAMYYCASRGGH
YGYALDYWGQGTLVTVSSGSTSGSGKPGSSEGSTKGEIVMTQTPATLSVS
AGERVTITCKASQSVSNDVTWYQQKPGQAPRLLIYSASNRYSGVPARFSG
SGYGTEFTFTISSVQSEDFAVYFCQQDYSSFGQGTKLEIKTSTTTPAPRP
PTPAPTIASQPLSLRPEACRPAAGGAVHTRGLDFACDIYIWAPLAGTCGV
LLLSLVITLYCRRLKIQVRKAAIASREKADAVYTGLNTRSQETYETLKHE
KPPQGSGSMVSKGEELFTGVVPILVELDGDVNGHKFSVSGEGEGDATYGK
LTLKFICTTGKLPVPWPTLVTTLTYGVQCFSRYPDHMKQHDFFKSAMPEG
YVQERTIFFKDDGNYKTRAEVKFEGDTLVNRIELKGIDFKEDGNILGHKL
EYNYNSHNVYIMADKQKNGIKVNFKIRHNIEDGSVQLADHYQQNTPIGDG
PVLLPDNHYLSTQSKLSKDPNEKRDHMVLLEFVTAAGITLGMDELYK

### Results

GD2 is a commonly overexpressed antigen in multiple solid tumors including melanoma, neuroblastoma, and small cell lung cancer (Suzuki & Cheung, Expert Opin Ther Targets 19:349-362 (2015); Nazha et al., Front Oncol., 10:1000 (2020)). A CAR targeting GD2 (disialoganglioside) was generated and introduced into mast cells by lentiviral transduction (FIGS. 3A and 3B). These CAR-mast cells were co-cultured with GD2+B16 cells. The activation of CAR-mast cells, as determined by the extracellular membrane expression of the degranulation marker CD107a, and the release of TNFα, were dependent on the presence of both CAR and GD2 (FIG. 3C), indicating an antigen-specific activation of CAR-mast cells.

To test the cytotoxicity of the GD2 CAR-mast cells, CAR-mast cells were co-cultured with GD2+ or GD2− B16 cells, using plain mast cells as a control. Luciferase assay revealed that CAR-mast cells showed much higher cytotoxicity towards GD2+B16 cells as compared to GD2-B16 cells (FIG. 3D). Furthermore, the cytokine and chemokine assay revealed effectors that were released by CAR-mast cells in responding to GD2 antigen. In addition to those secreted by CD19 CAR-mast cells (FIG. 3E), GD2 CAR-mast cells also secreted CXCL1, CCL5/17/20, and CXCL5 (FIG. 3E).

CAR mast cells (CAR+) killing was also tested over several rounds. Killing of pre-seeded MC38-GD2-mCherry-Luciferase cells (GD2+) at 0.01M after 24 hr coculture at 37° C. at the E:T of 0.008:1, 0.04:1, 0.2:1, 1:1, 5:1 respectively was assayed (FIG. 3F). At the end of each round the total supernatant is transferred to a new well pre-seeded with 0.01M fresh MC38-GD2-mCherry-Luciferase cells. Killing rate was calculated by (mean luciferase signal in control−luciferase signal after coculture)/mean luciferase signal in control*100%, where the control is 0.01M fresh MC38-GD2− mCherry-Luciferase cells treated with medium only.

### Example 3: Generation and Characterization of Mast Cell CARs Targeting GD2

### Materials and Methods

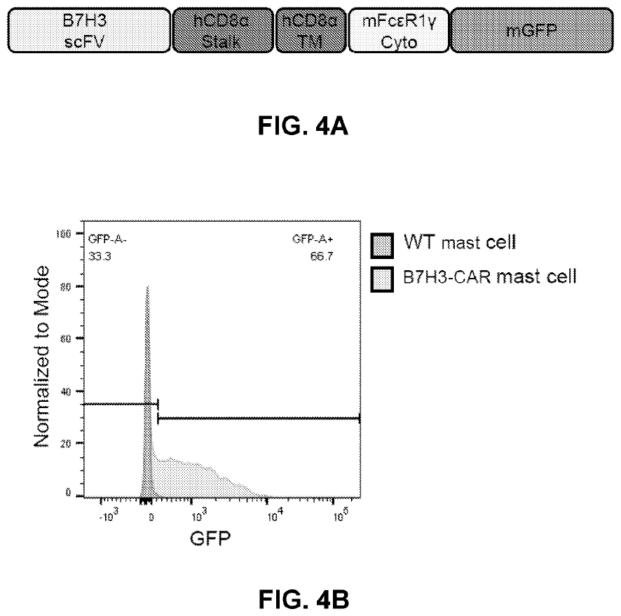

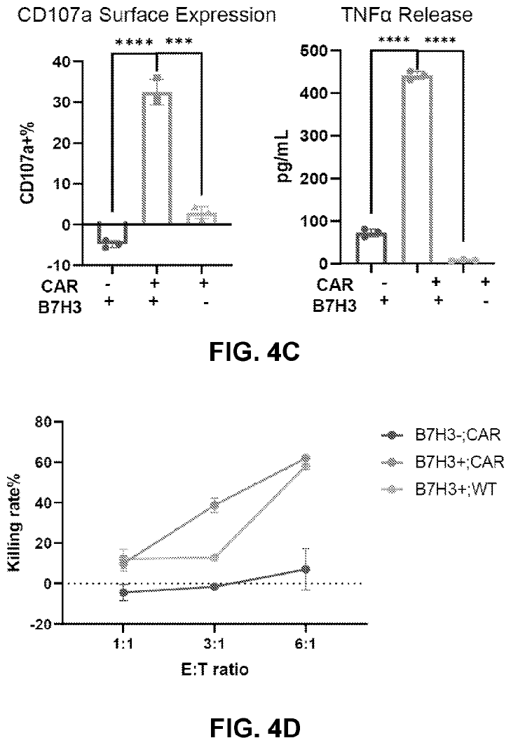

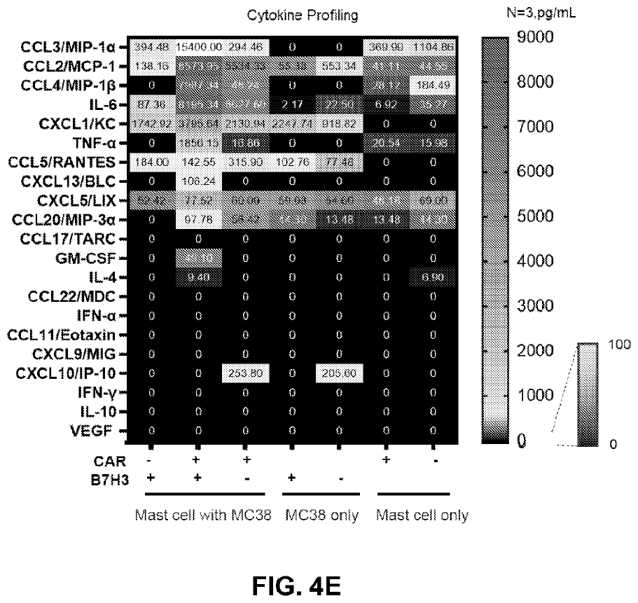

Mature mast cells were generated as described in Example 1 and illustrated in FIGS. 1A-1C. A CAR targeting B7H3(CD276) was constructed (FIG. 4A). B7H3 (CD276) is another common antigen associated with a variety of solid tumors (Liu, S. et al. Front Oncol 11:654684, (2021)). These B7H3 CAR-mast cells were cultured with MC38, a mouse colon cancer cell line, or MC38 overexpressing human B7H3 (FIG. 4B). Sequences for the primary elements of the B7H3 CAR are provided below.

a.
SP (Signal Peptide):
(SEQ ID NO: 1)
MALPVTALLLPLALLLHAARP
b.
HA tag:
(SEQ ID NO: 7)
YPYDVPDYAYPYDVPDYA
c.
anti-Human B7H3 scFv:
(SEQ ID NO: 9)
QVKLQQSGAELVKPGASVKLSCKASGYTFTNYDINWVRQRPEQGLEWIGW
IFPGDGSTQYNEKFKGKATLTTDTSSSTAYMQLSRLTSEDSAVYFCARQT
TATWFAYWGQGTTVTVSSDGGGSGGGGSGGGGSDIELTQSPTTLSVTPGD
RVSLSCRASQSISDYLHWYQQKSHESPRLLIKYASQSISGIPSRFSGSGS
GSDFTLSINSVEPEDVGVYYCQNGHSFPLTFGAGTKLELKQTS
d.
Mouse CD28 stalk and transmembrane domain:
(SEQ ID NO: 10)
IEVMYPPPYLDNEKSNGTIIHVKGKHLCPSPLFPGPSKPFWVLVVVGGVL
ACYSLLVTVAFIIFWV.
e.
Mouse FcεR1 gamma intracellular domain:
(SEQ ID NO: 5)
RLKIQVRKAAIASREKADAVYTGLNTRSQETYETLKHEKPPQGSGS
f.
mGFP:
(SEQ ID NO: 6)
MVSKGEELFTGVVPILVELDGDVNGHKFSVSGEGEGDATYGKLTLKFICT
TGKLPVPWPTLVTTLTYGVQCFSRYPDHMKQHDFFKSAMPEGYVQERTIF
FKDDGNYKTRAEVKFEGDTLVNRIELKGIDFKEDGNILGHKLEYNYNSHN
VYIMADKQKNGIKVNFKIRHNIEDGSVQLADHYQQNTPIGDGPVLLPDNH
YLSTQSKLSKDPNEKRDHMVLLEFVTAAGITLGMDELYK
g.
full B7H3 CAR-mast sequence
(SEQ ID NO: 24)
MALPVTALLLPLALLLHAARPYPYDVPDYAYPYDVPDYAQVKLQQSGAEL
VKPGASVKLSCKASGYTFTNYDINWVRQRPEQGLEWIGWIFPGDGSTQYN
EKFKGKATLTTDTSSSTAYMQLSRLTSEDSAVYFCARQTTATWFAYWGQG
TTVTVSSDGGGSGGGGSGGGGSDIELTQSPTTLSVTPGDRVSLSCRASQS
ISDYLHWYQQKSHESPRLLIKYASQSISGIPSRFSGSGSGSDFTLSINSV
EPEDVGVYYCQNGHSFPLTFGAGTKLELKQTSIEVMYPPPYLDNEKSNGT
IIHVKGKHLCPSPLFPGPSKPFWVLVVVGGVLACYSLLVTVAFIIFWVRL
KIQVRKAAIASREKADAVYTGLNTRSQETYETLKHEKPPQGSGSMVSKGE
ELFTGVVPILVELDGDVNGHKFSVSGEGEGDATYGKLTLKFICTTGKLPV
PWPTLVTTLTYGVQCFSRYPDHMKQHDFFKSAMPEGYVQERTIFFKDDGN
YKTRAEVKFEGDTLVNRIELKGIDFKEDGNILGHKLEYNYNSHNVYIMAD
KQKNGIKVNFKIRHNIEDGSVQLADHYQQNTPIGDGPVLLPDNHYLSTQS
KLSKDPNEKRDHMVLLEFVTAAGITLGMDELYK

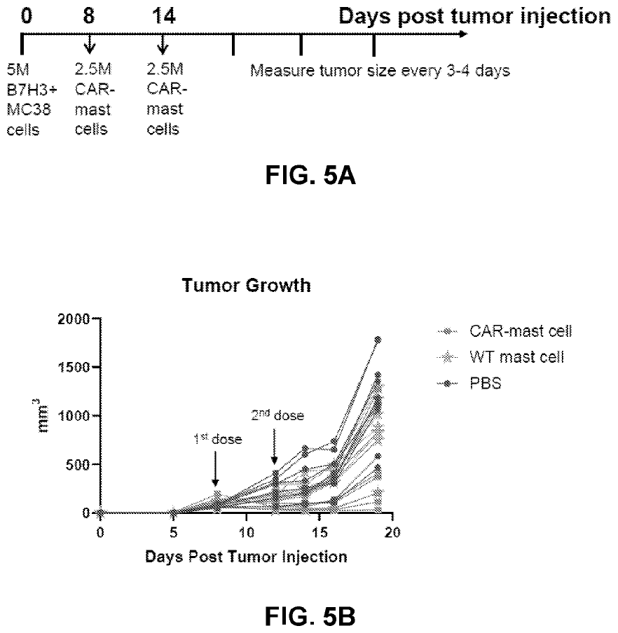

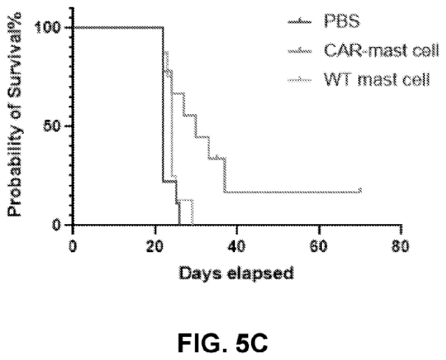

To determine the antitumor efficacy of CAR-mast cell in vivo, a MC38 xenograft model in immune-competent C57BL/6 mice was used. B7H3+MC38 cells were injected subcutaneously into the mice to form palpable tumors, which further received two doses of mast cells intratumorally at Day8 and Day14, respectively (FIG. 5A).

### Results

B7H3 (CD276 is another common antigen associated with a variety of solid tumors (Liu, S. et al. Front Oncol 11:654684, (2021)). A CAR targeting B7H3(CD276) was constructed and characterized (FIG. 4A). CAR-mast cells were activated in a B7H3-dependent manner, as reflected by the surface exposure of CD107a and the secretion of TNFα (FIG. 4C). Consistently, CAR-mast cells displayed a higher cytotoxicity towards B7H3+MC38 cells, as compared to plain MC38 cells (FIG. 4D). Moreover, CAR-mast cells specifically secreted CCL2/3/4, TNFα, CCL17/20, and CXCL13 (FIG. 4E). Taken together, the common chemokines secreted by CD19, GD2, or B7H3 CAR-mast cells include CCL2/3/4, which recruit monocytes, T cells and NK cells, and TNFα, which promotes cell death.

CAR-mast cells killing of a mixture of MC38-B7H3 cells (Antigen+) and MC38-mCherry-Luciferase cells (Antigen-) after 21 hr coculture at 37° C. at the E:T of 1:1, 3:1, 6:1 respectively with cancer cells total number at 0.02M was assayed (FIG. 4F). The percentage of Antigen− in the cancer cell mix was 10%, 30%, 60%, 100% respectively. Antigen− specific killing rate was calculated by (mean luciferase signal in control−luciferase signal after coculture)/mean luciferase signal in control*100%, where the control is cancer cells treated with medium only. The results demonstrated a bystander killing capacity of CAR-mast cells towards non antigen expressing cancer cells in a heterogeneous cancer population.

To determine the antitumor efficacy of CAR-mast cell in vivo, a MC38 xenograft model in immune-competent C57BL/6 mice was used. Immune-competent mice were used because, based on the above data, CAR-mast cells secrete chemokines that can recruit T and NK cells into tumor tissues, which is expected to contribute to tumor clearance. It was observed that CAR-mast cells inhibited tumor growth (FIG. 5B). Mice received the CAR-mast cells survived longer compared with those that received PBS or wild-type mast cells (FIG. 5C). Together, these data demonstrated that CAR-mast cells can kill solid tumors both in vivo and in vitro.

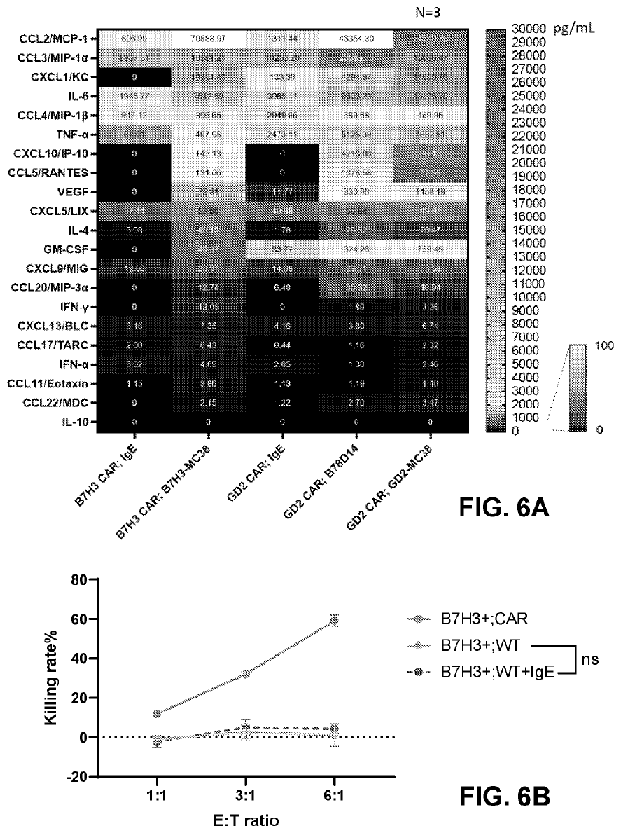

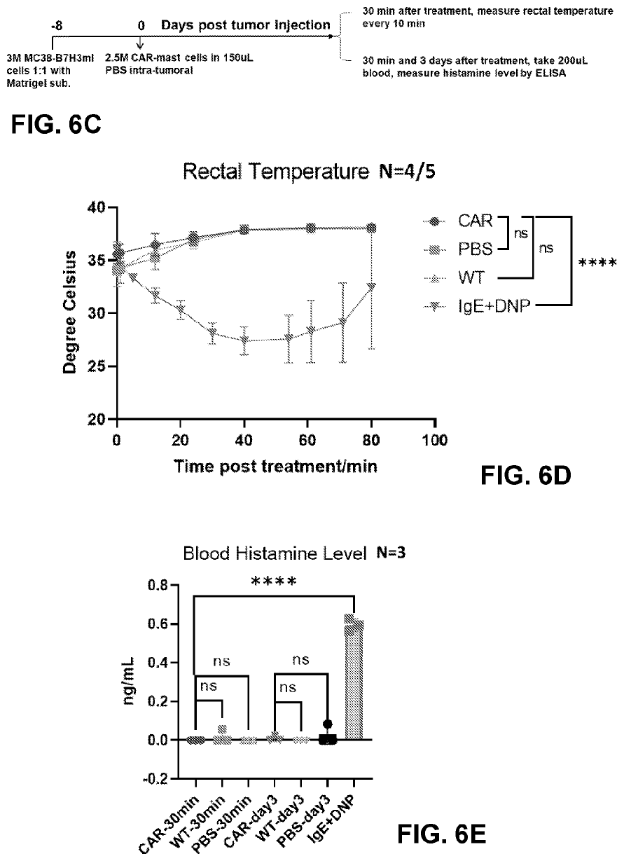

CAR-Mast cells were also investigated for and compared to allergic responses (e.g., IgE), B7-H3 CAR-mast cells (B7H3 CAR) or GD2 CAR-mast cells (GD2 CAR) were activated by either coculturing with MC38-B7H3-mCherry-Luciferase cells (B7H3-MC38) or MC38-GD2-mCherry-Luciferase cells (GD2-MC38) for 20 hr coculture at 37° C. at the E:T of 1:1 respectively, or by IgE where the CAR-mast cells are sensitized with 1 ug/mL anti-DNP IgE for 1 hr then cocultured with 20 ng/mL DNP for 20 hr at 37° C.; 100 μL supernatant was taken for cytokine profiling using Multiplexed Mouse Proinflammatory Chemokine Panel (Biolegend) or Mouse CRS Panel (Biolegend) (FIG. 6A). Results showed CAR mediated mast cell activation leads to higher release of multiple cytokines including CCL2, CCL3, IL-6 and TNFα.

Next, MC38-B7H3-mCherry-Luciferase cells (B7-H3+) were treated with either B7-H3 CAR mast cells (CAR), wildtype mast cells (WT) or IgE-activated wildtype mast cells (WT+IgE) for 21 hr at 37° C. at the E:T of 1:1, 3:1, 6:1 respectively with cancer cells total number at 0.02M. In the (WT+IgE) group, the mast cells were sensitized with 1 ug/mL anti-DNP IgE for 1 hr before cocultured with 20 ng/mL DNP and the cancer cells. Cancer cell killing rate is calculated by (mean luciferase signal in control−luciferase signal after coculture)/mean luciferase signal in control*100% (FIG. 6B), where the control is cancer cells treated with medium only. Results demonstrated a necessity of CAR-mediated activation for mast cells to perform direct cancer killing.

FIG. 6C is a diagram of mouse CAR-mast cell anaphylactic responses evaluation in mouse MC38-B7H3 tumor model. Timing of mast cell injections are indicated by the arrows. MC38-B7H3 ml are MC38-B7H3-mCherry-Luciferase cells.

Rectal temperature was taken with a digital thermometer every 10-12 min after treatment of CAR-mast cells (CAR, N=5), wildtype mast cells (WT, N=5) or PBS vehicle (PBS, N=5) (FIG. 6D). Mice in the (IgE+DNP, N=4) group were sensitized i.p. with 10 ug anti-TNP IgE in 100 uL PBS 16 hr earlier, then challenged i.p. with 50 ug TNP-OVA in 100 ul PBS as the same time as other treatments.

At 30 min or day 3 after treatment, mice were humanely killed by decapitation, and blood samples were obtained after a cardiac puncture to measure the plasma histamine concentration with a histamine enzyme immunoassay kit (FIG. 6E).

Together, FIG. 6D and FIG. 6E demonstrated no significant anaphylactic response will be observed following CAR-mast cell therapy with described treatment regime.

### Example 4: Generation and Characterization of Mast Cell CARS Targeting HER2

### Materials and Methods

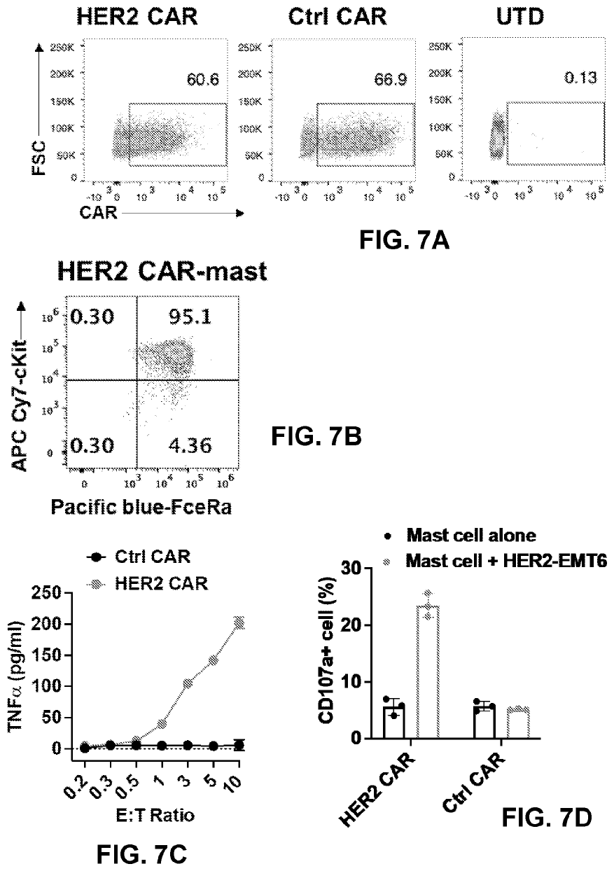

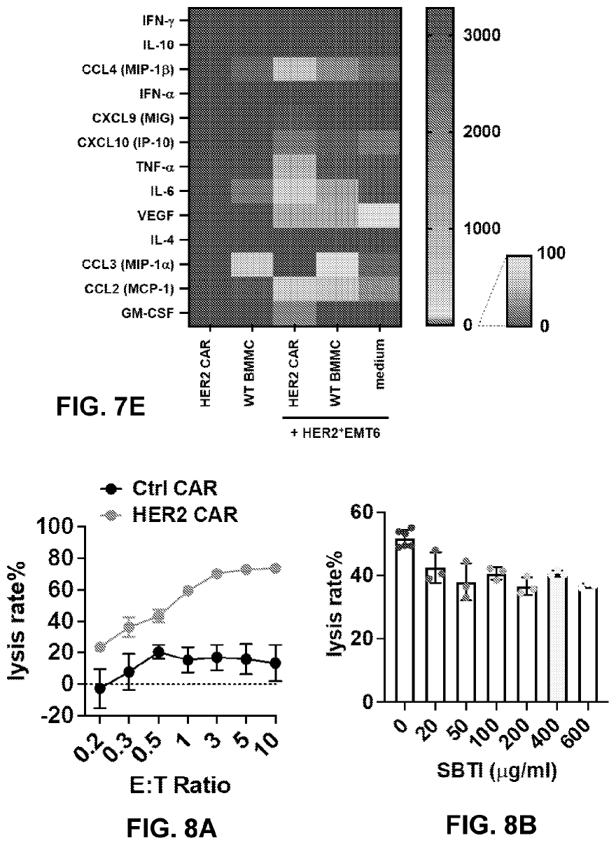

A mast-cell CAR targeting HER2 was constructed containing the anti-HER2 single-chain variable fragment, the stalk and transmembrane domain from CD8α, cytoplasmic domain of FcεRI gamma chain, and a monomer GFP for CAR labelling. This HER2 CAR was introduced into mature mouse mast cells by lentiviral transduction (FIG. 7A). The maturity of mast cells was validated by the expressions of mast cell maturation markers c-kit and FcεRI (FIG. 7B). To test the cytotoxicity, These HER2 CAR-mast cells were co-cultured with HER2-ectopically expressing EMT6 cells (HER2-EMT6), which are mouse syngeneic triple-negative breast cancer cells. CD19 CAR-mast cells were used as negative controls. Sequences for the primary elements of the HER2 CAR are provided below.

a.
SP (Signal Peptide):
(SEQ ID NO: 33)
METDTLLLWVLLLWVPGSTGD
b.
anti-HER2 scFv:
(SEQ ID NO: 34)
KYLLPTAAAGLLLLAAQPAMAQVQLVQSGAEVKKPGESLKISCKGSGYSF
TSYWIAWVRQMPGKGLEYMGLIYPGDSDTKYSPSFQGQVTISVDKSVSTA
YLQWSSLKPSDSAVYFCARHDVGYCTDRTCAKWPEYFQHWGQGTLVTVSS
GGGGSGGGGSGGGGSQSVLTQPPSVSAAPGQKVTISCSGSSSNIGNNYVS
WYQQLPGTAPKLLIYDHTNRPAGVPDRFSGSKSGTSASLAISGFRSEDEA
DYYCASWDYTLSGWVFGGGTKLTVLGGSGS
c.
Myc tag:
(SEQ ID NO: 36)
EQKLISEEDLTS
d.
Human CD8α Stalk:
(SEQ ID NO: 3)
TTTPAPRPPTPAPTIASQPLSLRPEACRPAAGGAVHTRGLDFACD
e.
Human CD8α transmembrane domain:
(SEQ ID NO: 4)
IYIWAPLAGTCGVLLLSLVITLYCR
f.
Mouse FcεR 1 gamma intracellular domain:
(SEQ ID NO: 5)
RLKIQVRKAAIASREKADAVYTGLNTRSQETYETLKHEKPPQGSGS
g.
mGFP:
(SEQ ID NO: 6)
MVSKGEELFTGVVPILVELDGDVNGHKFSVSGEGEGDATYGKLTLKFICT
TGKLPVPWPTLVTTLTYGVQCFSRYPDHMKQHDFFKSAMPEGYVQERTIF
FKDDGNYKTRAEVKFEGDTLVNRIELKGIDFKEDGNILGHKLEYNYNSHN
VYIMADKQKNGIKVNFKIRHNIEDGSVQLADHYQQNTPIGDGPVLLPDNH
YLSTQSKLSKDPNEKRDHMVLLEFVTAAGITLGMDELYK
h.
full HER2 CAR-mast sequence
(SEQ ID NO: 35)
METDTLLLWVLLLWVPGSTGDKYLLPTAAAGLLLLAAQPAMAQVQLVQSG
AEVKKPGESLKISCKGSGYSFTSYWIAWVRQMPGKGLEYMGLIYPGDSDT
KYSPSFQGQVTISVDKSVSTAYLQWSSLKPSDSAVYFCARHDVGYCTDRT
CAKWPEYFQHWGQGTLVTVSSGGGGSGGGGSGGGGSQSVLTQPPSVSAAP
GQKVTISCSGSSSNIGNNYVSWYQQLPGTAPKLLIYDHTNRPAGVPDRES
GSKSGTSASLAISGFRSEDEADYYCASWDYTLSGWVFGGGTKLTVLGGSG
SEQKLISEEDLTSTTTPAPRPPTPAPTIASQPLSLRPEACRPAAGGAVHT
RGLDFACDIYIWAPLAGTCGVLLLSLVITLYCRRLKIQVRKAAIASREKA
DAVYTGLNTRSQETYETLKHEKPPQGSGSMVSKGEELFTGVVPILVELDG
DVNGHKFSVSGEGEGDATYGKLTLKFICTTGKLPVPWPTLVTTLTYGCQC
FSRYPDHMKQHDFFKSAMPEGYVQERTIFFKDDGNYKTRAEVKFEGDTLV
NRIELKGIDFKEDGNILGHKLEYNYNSHNVYIMADKQKNGIKVNFKIRHN
IEDGSVQLADHYQQNTPIGDGPVLLPDNHYLSTQSKLSKDPNEKRDHMVL
LEFVTAAGITLGMDELYK

### Results

Experiments were designed to investigate the expression of the CAR construct, the formation of transduced cells as CAR-mast cells, and to test CAR-mast cells activity. CAR expression was confirmed on CAR-mast cells by flow cytometry, and compared to Control CD19 CAR-mast cells (introduced above). The results are shown in FIG. 7A. FIG. 7B shows high surface expressions of mast cell lineage markers (e.g., cKit, FcεRa) on HER2 CAR-mast cells, indicating these cells are mature mast cells. HER2 CAR-mast cells were specifically activated by HER2-EMT6 cells, as reflected by the increased surface exposure of CD107a (FIG. 7C) and TNFα production (FIG. 7D) after 48 hr of coculture with HER2-EMT6. FIG. 7E shows the cytokine and chemokine released by HER2 CAR-mast cells when cocultured with and without HER2-EMT6 cells for 48 hr. Similar with other CAR-mast cells introduced above, HER2 CAR-mast cells encountering HER2-EMT6 cells produced high amounts of CCL2, CCL3, CCL4, IL-6, TNFα and GM-CSF.

Cytotoxicity of HER2 CAR-mast cells were also investigated. FIG. 8A show cytotoxicity of HER2 CAR-mast cells against HER2-EMT6 cells across a broad range of effector-to-target ratios (FIG. 8A). The cytotoxicity was constrained in the presence of varying concentrations of Soybean Trypsin Inhibitor (SBTI) (FIG. 8B), which shows that cell-killing activity is dependent on tryptase activity.

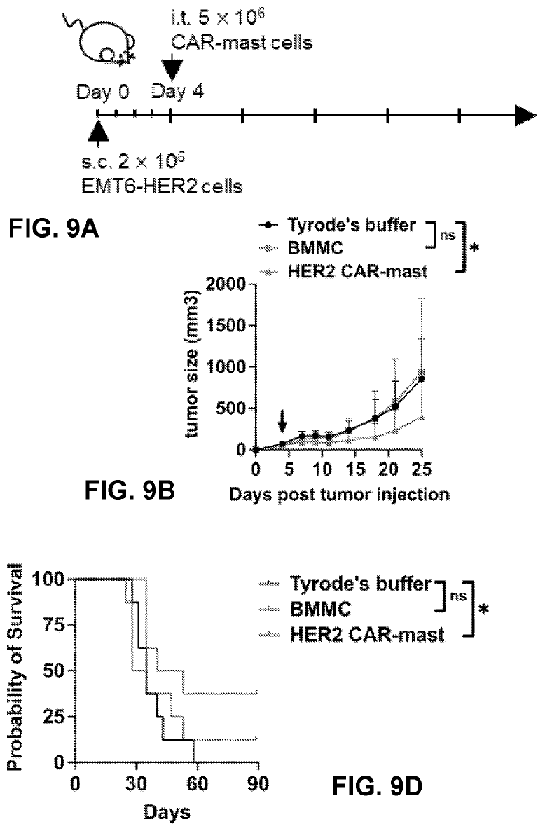

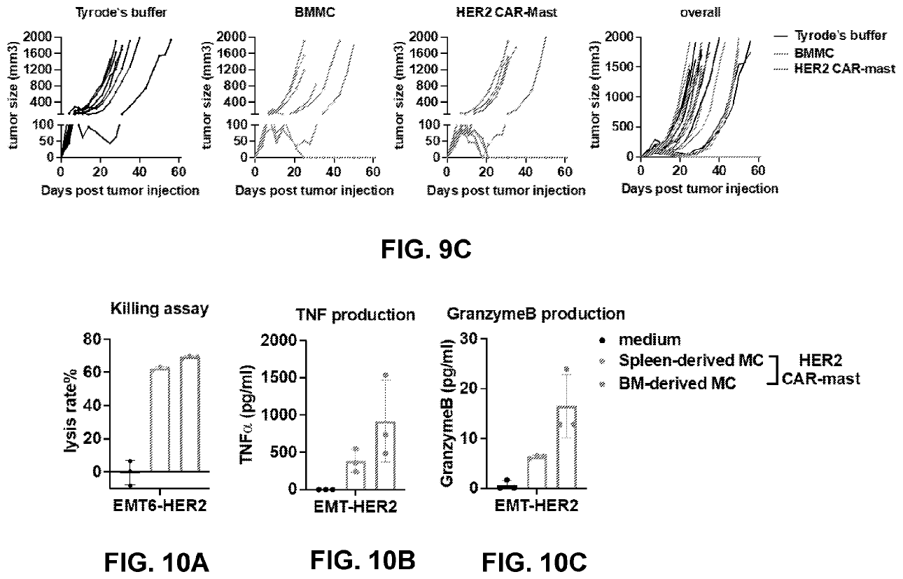

FIG. 9A is a schema of an in vivo assay during which FFluc+-HER2-EMT6-engrafted mouse received one dose (5×10⁶ cells in 100 ul Tyrode's buffer) of CAR-mast cells, WT bone marrow-derived mast cells, or Tyrode's buffer by intratumoral injection (n=8 mice per group). Results are shown in FIGS. 9B-9D. Average tumor burden of mice through a 25-day period after FFluc+-HER2-EMT6 cells inoculation is shown in FIG. 9B. Tumor-bearing mice receiving CAR-mast cells showed the lowest tumor burden as compared the WT mast cell-treated and Tyrode's buffer-treated mice, associating with improved response (FIG. 9C) and survival outcomes (FIG. 9D).

Additional investigation shows that CAR-mast cells generated from spleen or bone marrow-derived mast cells have comparable cytotoxicity and activation. Results are presented in bar graphs FIGS. 10A-10C, where the bars, from left to right, represent medium (control), spleen derived HER2 CAR-mast cells (MC), and bone marrow (BM) derived HER2 CAR-mast cells (MC). Cytotoxicity of HER2 CAR-mast cells was determined after 48 hr of coculture with HER2-EMT6 cells (E:T=5:1) (FIG. 10A). TNFα production (FIG. 10B) and Granzyme B production (FIG. 10C) in the coculture supernatant were measured by ELISA. Spleen and bone marrow-derived HER2 CAR-mast cells showed similar cytotoxicity, although bone marrow-derived cells yielded higher levels of TNFα and Granzyme B than the spleen-derived mast cells.

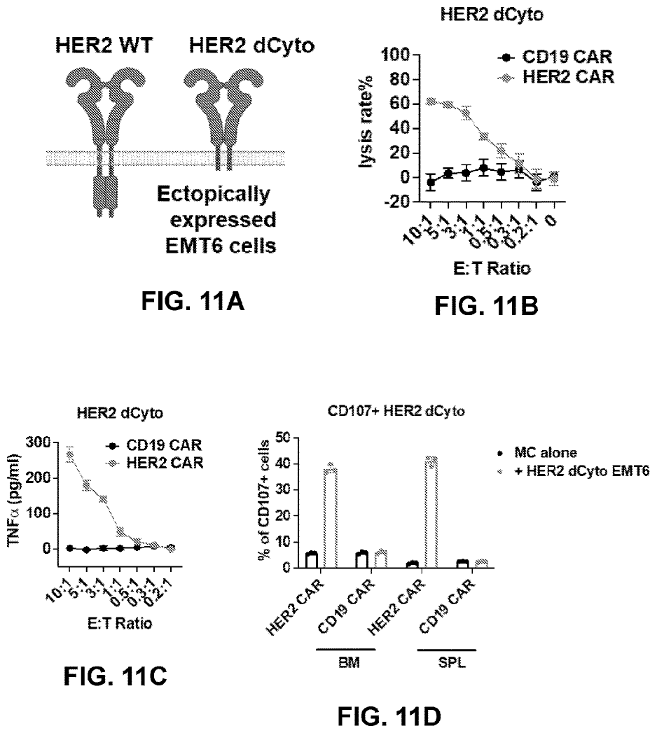

Additional experiments were developed to investigate if HER2 CAR-mast cell cytotoxicity is contingent on tumor intrinsic HER2 signaling. Results show it is not. A signaling-inert mutant, the cytoplasmic domain-truncated HER2 (HER2 dCyto), was constructed and expressed in EMT6 cells (FIG. 11A). HER2 CAR-mast cells showed antigen-specific killing activity against HER2-dCyto-expressing EMT6 cells (FIG. 11B), as well as antigen-specific activation as demonstrated by the increased TNFα production (FIG. 11C) and elevated surface level of CD107 (FIG. 11D) when compared to the control CD19 CAR-mast cells. CAR-mast cells generated from spleen-derived (SPL) mast cells or bone marrow-derived (BM) mast cells demonstrate comparably activation status (FIG. 11D).

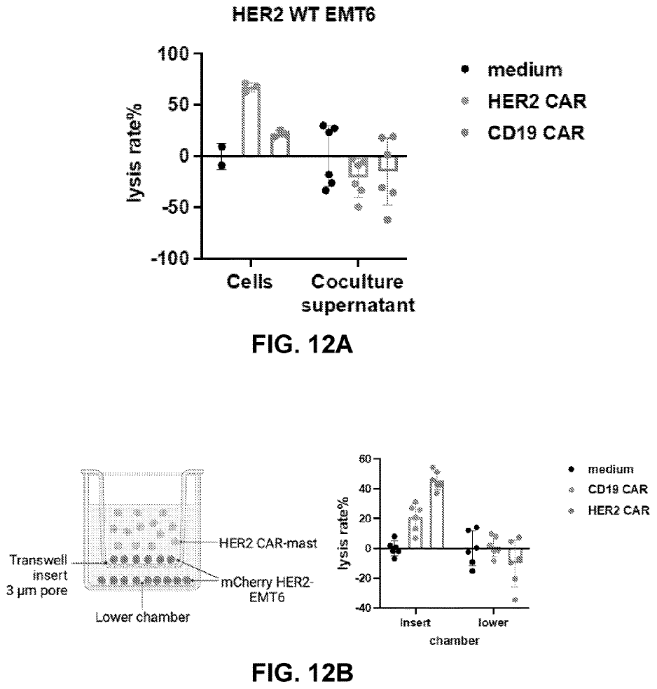

Additional experiments were designed to determine if CAR-mast cell cytotoxicity against EMT6 cells involves cell-cell contacts, and the results presented in FIGS. 12A-12B show that it does. CAR-mast cells was cocultured with HER2-EMT6 cells (E:T=5:1) for 48 hr (“cells”). Supernatant from coculture (“coculture supernatant”) were added to a new batch of HER2-EMT6 cells. Lysis rates against HER2-EMT6 cells were determined by luciferase assay are presented in FIG. 12A. From left to right in clustered for both “cells” and “coculture supernatant” bars correspond to medium (control), HER2 CAR, and CD19 CAR. Results showed that coculture supernatant failed to induce cytotoxicity of HER2-EMT6 cells, indicating the killing of CAR-mast cells likely utilizes cell-cell contacts. Similar results were obtained from a transwell setting, where HER2-EMT6 cells were seeded on both the insert membrane (3 um pore) and lower bottom of the same transwell, while HER 2 CAR-mast cells were added to the insert (E:T=5:1) and cultured for 48 hr (FIG. 12B, left). Cytotoxicity against HER2-EMT6 cells on the insert membrane and lower bottom were determined by luciferase assay. It showed that HER2 CAR-mast exhibited a potent cytotoxic activity toward the HER2-EMT6 cells in close proximity, but their efficacy diminished for cells located at a greater distance (FIG. 12B, right).

### REFERENCES

1 Feng, K. et al. Chimeric antigen receptor-modified T cells for the immunotherapy of patients with EGFR-expressing advanced relapsed/refractory non-small cell lung cancer. Sci China Life Sci 59, 468-479, (2016).
2 Beatty, G. L. et al. Mesothelin-specific chimeric antigen receptor mRNA-engineered T cells induce anti-tumor activity in solid malignancies. Cancer Immunol Res 2, 112-120, (2014).
3 Tchou, J. et al. Safety and Efficacy of Intratumoral Injections of Chimeric Antigen Receptor (CAR) T Cells in Metastatic Breast Cancer. Cancer Immunol Res 5, 1152-1161, (2017).
4 Kakarla, S. & Gottschalk, S. CAR T cells for solid tumors: armed and ready to go? Cancer J 20, 151-155, (2014).
5 Oliver, A. J. et al. Tissue-Dependent Tumor Microenvironments and Their Impact on Immunotherapy Responses, Front Immunol 9, 70, (2018).
6 Sadelain, M., Riviere, I. & Riddell, S. Therapeutic T cell engineering. Nature 545, 423-431, (2017).
7 Gordon, K. S. et al. Screening for CD19-specific chimaeric antigen receptors with enhanced signalling via a barcoded library of intracellular domains. Nat Biomed Eng 6, 855-866, (2022).
8 Goodman, D. B. et al. Pooled screening of CAR T cells identifies non-native signaling domains for next-generation immunotherapies. bioRxiv (2021).
9 Klichinsky, M. et al. Human chimeric antigen receptor macrophages for cancer immunotherapy. Nat Biotechnol, (2020).
10 Liu, E. et al. Use of CAR-Transduced Natural Killer Cells in CD19-Positive Lymphoid Tumors. N Engl J Med 382, 545-553, (2020).
11 Chockley, P. J., Ibanez-Vega, J., Krenciute, G., Talbot, L. J. & Gottschalk, S. Synapse-tuned CARs enhance immune cell anti-tumor activity. Nat Biotechnol, (2023).
12 Qian, B. Z. & Pollard, J. W. Macrophage diversity enhances tumor progression and metastasis. Cell 141, 39-51, doi:10.1016/j.cell.2010.03.014 (2010).
13 Moon, T. C., Befus, A. D. & Kulka, M. Mast cell mediators: their differential release and the secretory pathways involved. Front Immunol 5, 569, (2014).
14 Wernersson, S. & Pejler, G. Mast cell secretory granules: armed for battle. Nat Rev Immunol 14, 478-494, doi:10.1038/nri3690 (2014).
15 Iskarpatyoti, J. A. et al. Mast cell regranulation requires a metabolic switch involving mTORC1 and a glucose-6-phosphate transporter. Cell Rep 40, 111346, doi:10.1016/j.celrep.2022.111346 (2022).
16 Xiang, Z., Block, M., Lofman, C. & Nilsson, G. IgE-mediated mast cell degranulation and recovery monitored by time-lapse photography. J Allergy Clin Immunol 108, 116-121, doi:10.1067/mai.2001.116124 (2001).
17 St John, A. L. & Abraham, S. N. Innate immunity and its regulation by mast cells. J Immunol 190, 4458-4463, doi:10.4049/jimmunol.1203420 (2013).
18 Galli, S. J., Gaudenzio, N. & Tsai, M. Mast Cells in Inflammation and Disease: Recent Progress and Ongoing Concerns. Annu Rev Immunol 38, 49-77, doi:10.1146/annurev-immunol-071719-094903 (2020).
19 Fu, H. et al. Tumor Infiltrating Mast Cells (TIMs) Confers a Marked Survival Advantage in Nonmetastatic Clear-Cell Renal Cell Carcinoma. Ann Surg Oncol 24, 1435-1442, doi:10.1245/s10434-016-5702-5 (2017).
20 Hempel, H. A. et al. Low Intratumoral Mast Cells Are Associated With a Higher Risk of Prostate Cancer Recurrence. Prostate 77, 412-424, doi:10.1002/pros.23280 (2017).
21 Dantas, R. C. M. et al. Evaluation of Mast Cell Density in the Tumor Microenvironment in Oral Epithelial Dysplasia and Oral Squamous Cell Carcinoma. Appl Immunohistochem Mol Morphol 25, e83-e88, (2017).
22 Shikotra, A., Ohri, C. M., Green, R. H., Waller, D. A. & Bradding, P. Mast cell phenotype, TNFalpha expression and degranulation status in non-small cell lung cancer. Sci Rep 6, 38352, doi:10.1038/srep38352 (2016).
23 Malfettone, A. et al. High density of tryptase-positive mast cells in human colorectal cancer: a poor prognostic factor related to protease-activated receptor 2 expression. J Cell Mol Med 17, 1025-1037, doi:10.1111/jcmm.12073 (2013).
24 Ghouse, S. M. et al. Although Abundant in Tumor Tissue, Mast Cells Have No Effect on Immunological Micro-milieu or Growth of HPV-Induced or Transplanted Tumors. Cell Rep 22, 27-35, doi:10.1016/j.celrep.2017.12.010 (2018).
25 Kaesler, S. et al. Targeting tumor-resident mast cells for effective anti-melanoma immune responses. JCI Insight 4, doi:10.1172/jci.insight.125057 (2019).
26 Fereydouni, M. et al. Human Tumor Targeted Cytotoxic Mast Cells for Cancer Immunotherapy. Front Oncol 12, 871390, doi:10.3389/fonc.2022.871390 (2022).
27 Ohkawara, Y. et al. Human lung mast cells and pulmonary macrophages produce tumor necrosis factor-alpha in sensitized lung tissue after IgE receptor triggering. Am J Respir Cell Mol Biol 7, 385-392, doi:10.1165/ajrcmb/7.4.385 (1992).
28 Plotkin, J. D. et al. Human Mast Cells From Adipose Tissue Target and Induce Apoptosis of Breast Cancer Cells. Front Immunol 10, 138, doi:10.3389/fimmu.2019.00138 (2019).
29 Strik, M. C. et al. Human mast cells produce and release the cytotoxic lymphocyte associated protease granzyme B upon activation. Mol Immunol 44, 3462-3472, doi:10.1016/j.molimm.2007.03.024 (2007).
30 Sektioglu, I. M. et al. Basophils Promote Tumor Rejection via Chemotaxis and Infiltration of CD8+ T Cells. Cancer Res 77, 291-302, doi:10.1158/0008-5472.CAN-16-0993 (2017).
31 Wu, Z. et al. Mast cell FcepsilonRI-induced early growth response 2 regulates CC chemokine ligand 1-dependent CD4+ T cell migration. J Immunol 190, 4500-4507, doi:10.4049/jimmunol.1203158 (2013).
32 Padawer, J. Mast cells: extended lifespan and lack of granule turnover under normal in vivo conditions. Exp Mol Pathol 20, 269-280, doi:10.1016/0014-4800(74)90059-8 (1974).
33 Slutsky, B., Jarvis, D., Bibb, P., Feldberg, R. S. & Cochrane, D. E. Viability and recovery from degranulation of isolated rat peritoneal mast cells. Exp Cell Res 168, 63-78, doi:10.1016/0014-4827(87)90416-2 (1987).
34 Rieckmann, J. C. et al. Social network architecture of human immune cells unveiled by quantitative proteomics. Nat Immunol 18, 583-593, (2017).
35 Zeng, L., Palaia, I., Saric, A. & Su, X. PLCgamma1 promotes phase separation of T cell signaling components. J Cell Biol 220, doi:10.1083/jcb.202009154 (2021).
36 Su, X. et al. Phase separation of signaling molecules promotes T cell receptor signal transduction. Science 352, 595-599, doi:10.1126/science.aad9964 (2016).
37 Xiao, Q. et al. Size-dependent activation of CAR-T cells. Sci Immunol 7, eab13995, doi:10.1126/sciimmunol.ab13995 (2022).
38 Dong, R. et al. Rewired signaling network in T cells expressing the chimeric antigen receptor (CAR). EMBO J 39, e104730, doi:10.15252/embj.2020104730 (2020).
39 Dong, M. B. et al. Systematic Immunotherapy Target Discovery Using Genome-Scale In Vivo CRISPR Screens in CD8 T Cells. Cell 178, 1189-1204 e1123, doi:10.1016/j.cell.2019.07.044 (2019).
40 Ye, L. et al. A genome-scale gain-of-function CRISPR screen in CD8 T cells identifies proline metabolism as a means to enhance CAR-T therapy. Cell Metab 34, 595-614 e514, doi:10.1016/j.cmet.2022.02.009 (2022).
41 Gilfillan, A. M. & Tkaczyk, C. Integrated signalling pathways for mast-cell activation. Nat Rev Immunol 6, 218-230, doi:10.1038/nri1782 (2006).
42 Suzuki, M. & Cheung, N. K. Disialoganglioside GD2 as a therapeutic target for human diseases. Expert Opin Ther Targets 19, 349-362, (2015).
43 Nazha, B., Inal, C. & Owonikoko, T. K. Disialoganglioside GD2 Expression in Solid Tumors and Role as a Target for Cancer Therapy. Front Oncol 10, 1000, (2020).
44 Lin, S., Cicala, C., Scharenberg, A. M. & Kinet, J. P. The Fc(epsilon)RIbeta subunit functions as an amplifier of Fc(epsilon)RIgamma-mediated cell activation signals. Cell 85, 985-995, doi:10.1016/s0092-8674(00)81300-8 (1996).
45 Kohno, M., Yamasaki, S., Tybulewicz, V. L. & Saito, T. Rapid and large amount of autocrine IL-3 production is responsible for mast cell survival by IgE in the absence of antigen. Blood 105, 2059-2065, doi:10.1182/blood-2004-07-2639 (2005).
46 Davenport, A. J. et al. Chimeric antigen receptor T cells form nonclassical and potent immune synapses driving rapid cytotoxicity. Proc Natl Acad Sci USA 115, (2018).
47 Liu, D. et al. The Role of Immunological Synapse in Predicting the Efficacy of Chimeric Antigen Receptor (CAR) Immunotherapy. Cell Commun Signal 18, 134, doi:10.1186/s12964-020-00617-7 (2020).
48 Xiong, W. et al. Immunological Synapse Predicts Effectiveness of Chimeric Antigen Receptor Cells. Mol Ther 29, 1349-1351, (2021).
49 Naghizadeh, A. et al. In vitro machine learning-based CAR T immunological synapse quality measurements correlate with patient clinical outcomes. PLoS Comput Biol 18, e1009883, doi:10.1371/journal.pcbi.1009883 (2022).
50 Libby, K. A. & Su, X. Imaging Chimeric Antigen Receptor (CAR) Activation. Methods Mol Biol 2111, 153-160, doi:10.1007/978-1-0716-0266-9_13 (2020).
51 Liu, S. et al. The Role of CD276 in Cancers. Front Oncol 11, 654684, doi:10.3389/fonc.2021.654684 (2021).
52 Joulia, R. et al. Mast cells form antibody-dependent degranulatory synapse for dedicated secretion and defense. Nat Commun 6, 6174, (2015).
53 Eyquem, J. et al. Targeting a CAR to the TRAC locus with CRISPR/Cas9 enhances tumor rejection. Nature 543, 113-117, doi:10.1038/nature21405 (2017).
54 Schmetzer, O. et al. A novel method to generate and culture human mast cells: Peripheral CD34+ stem cell-derived mast cells (PSCMCs). J Immunol Methods 413, 62-68, (2014).
55 Lehmann, M. H. et al. CCL2 expression is mediated by type I IFN receptor and recruits NK and T cells to the lung during MVA infection. J Leukoc Biol 99, 1057-1064, (2016).
56 Allen, F. et al. CCL3 augments tumor rejection and enhances CD8(+) T cell infiltration through NK and CD103(+) dendritic cell recruitment via IFNgamma. Oncoimmunology 7, e1393598, (2018).
57 Bougherara, H. et al. Real-Time Imaging of Resident T Cells in Human Lung and Ovarian Carcinomas Reveals How Different Tumor Microenvironments Control T Lymphocyte Migration. Front Immunol 6, 500, (2015).
58 Becker, J. C. et al. Mouse models for melanoma: a personal perspective. Exp Dermatol 19, 157-164, (2010).
59 Wang, J. et al. UV-induced somatic mutations elicit a functional T cell response in the YUMMER1.7 mouse melanoma model. Pigment Cell Melanoma Res 30, 428-435, (2017).

It is understood that the disclosed method and compositions are not limited to the particular methodology, protocols, and reagents described as these can vary. It is also to be understood that the terminology used herein is for the purpose of describing particular embodiments only, and is not intended to limit the scope of the present invention which will be limited only by the appended claims.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A fusion polypeptide comprising
(a) a mast cell intracellular signaling domain; and
(b) an amino acid sequence that is heterologous to the mast cell intracellular signaling domain.

2. The polypeptide of claim 1, wherein the amino acid sequence of (a) comprises the intracellular domain of IgE receptor (FcRI), a cytokine receptor (c-KIT), a G-protein-coupled receptor, or a toll-like receptor expressed in mast cells, or functional fragment or variant thereof.

3. The polypeptide of claim 22 comprising an ITAM sequence.

4. The polypeptide of claim 3, wherein the ITAM sequence comprises DGVYTGLSTRNQETYETLKHE (SEQ ID NO:11) or a functional variant thereof or a variant thereof having an amino acid sequence at least 70%, 75%, 80%, 85%, 90%, 95%, or 99% identical to SEQ ID NO:11.

5. The polypeptide of claim 1, wherein the amino acid sequence of (a) comprises the amino acid sequence of any one of SEQ ID NOS:11-21, or a functional fragment thereof, or a variant thereof having an amino acid sequence at least 70%, 75%, 80%, 85%, 90%, 95%, or 99% identical to any one of SEQ ID NOS:11-21.

6. The polypeptide of claim 1, wherein (b) comprises one or more of an extracellular domain, transmembrane domain, and/or a further intracellular domain.

7. The polypeptide of claim 6, wherein the polypeptide is a chimeric antigen receptor (CAR).

8. The polypeptide of claim 7, wherein the CAR comprises an intracellular domain comprising (a), a transmembrane domain, and an extracellular domain.

9. The polypeptide of claim 8, wherein (b) comprises a transmembrane domain and optionally stalk of CD8α, optionally of SEQ ID NO:4 or fragment or variant thereof with at least 70% sequence identity thereto, or CD28, optionally SEQ ID NO:10 or fragment or variant thereof with at least 70% sequence identity thereto.

10. The polypeptide of claim 9, wherein the CAR is specific for an antigen selected from the group consisting of a cancer antigen, an inflammatory disease antigen, a neuronal disorder antigen, HIV/AIDS, a diabetes antigen, a cardiovascular disease antigen, an infectious disease antigen (including a viral antigen, a protozoan antigen, a bacterial antigen, and an allergen), an autoimmune disease antigen and an autoimmune disease antigen, or combinations thereof.

11. The polypeptide of claim 10, wherein the CAR targets one or more antigens selected from the group consisting of B7H3, HER2, CD19, GD2, AFP, AKAP 4, ALK, Androgen receptor, B7H3, BCMA, Bcr Abl, BORIS, Carbonic, CD123, CD138, CD174, CD20, CD22, CD30, CD33, CD38, CD80, CD86, CEA, CEACAM5, CEACAM6, Cyclin, CYP1B1, EBV, EGFR, EGFR806, EGFRvIII, EpCAM, EphA2, ERG, ETV6 AML, FAP, Fos related antigen1, Fucosyl, fusion, GD3, GloboH, GM3, gp100, GPC3, HER 2/neu, HMWMAA, HPV E6/E7, hTERT, Idiotype, IL12, IL13RA2, IM19, IX, LCK, Legumain, IgK, LMP2, MAD CT 1, MAD CT 2, MAGE, MelanA/MART1, Mesothelin, MET, ML IAP, MUC1, Mutant p53, MYCN, NA17, NKG2D L, NY BR 1, NY ESO 1, NY ESO 1, OY TES1, p53, Page4, PAP, PAX3, PAX5, PD L1, PDGFR β, PLAC1, Polysialic acid, Proteinase3 (PR1), PSA, PSCA, PSMA, Ras mutant, RGS5, RhoC, ROR1, SART3, sLe(a), Sperm protein 17, SSX2, STn, Survivin, Tie2, Tn, TRP 2, Tyrosinase, VEGFR2, WT1, XAGE, Claudin-6, Claudin-18.2 and CD70.

12. The polypeptide of claim 11, wherein the antigen is a cancer antigen selected from the group consisting of B7H3, HER2, CD19, GD2, 41BB, 5T4, adenocarcinoma antigen, alpha fetoprotein, BAFF, B lymphoma cell, C242 antigen, CA 125, carbonic anhydrase 9 (CA IX), C MET, CCR4, CD152, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNT0888, CTLA 4, DR5, EGFR, EpCAM, CD3, FAP, fibronectin extra domain B, folate receptor 1, GD3 ganglioside, glycoprotein 75, GPNMB, HER2/neu, HGF, human scatter factor receptor kinase, IGF 1 receptor, IGF I, IgG1, L1 CAM, IL 13, IL 6, insulin-like growth factor I receptor, integrin α5β1, integrin αvβ3, MORAb 009, MS4A1, MUC1, mucin CanAg, N glycolylneuraminic acid, NPC 1C, PDGF R a, PDL192, phosphatidylserine, prostatic carcinoma cells, RANKL, RON, ROR1, SCH 900105, SDC1, SLAMF7, TAG 72, tenascin C, TGF beta 2, TGF 3, TRAIL R1, TRAIL R2, tumor antigen CTAA16.88, VEGF A, VEGFR 1, VEGFR2, and vimentin.

13. The polypeptide of claim 12, wherein the CAR comprising an antigen binding domain, optionally wherein the antigen-binding domain is anti-B7H3, anti-HER2, anti-CD19, or anti-GD2, optionally of SEQ ID NOS:9, 34, 2, or 8, respectfully, or a fragment or variant thereof comprising the complementary determining regions (CDRs) thereof.

14. A nucleic acid comprising a nucleic acid encoding the polypeptide of claim 1.

15.-22. (canceled)

23. An isolated cell comprising the nucleic acid of claim 14.

24.-26. (canceled)

27. The isolated cell of claim 23, wherein the polypeptide is a CAR.

28. The isolated cell of claim 27, wherein the intracellular domain of the CAR comprises the amino acid sequence of any one of SEQ ID NOS:11-21 or a fragment or variant thereof with at least 70% sequence identity thereto.

29. A population of cells derived by expanding the cell of claim 23.

30. A pharmaceutical composition comprising the population of cells of claim 29 and a pharmaceutically acceptable buffer, carrier, diluent, or excipient.

31. A method of treating a subject having a disease, disorder, or condition comprising administering to the subject an effective amount of the pharmaceutical composition of claim 30.

32.-40. (canceled)


SEQUENCE LISTING (36 sequences)

SEQ ID NO: 1 - protein, 21 aa, synthetic construct
MALPVTALLLPLALLLHAARP

SEQ ID NO: 2 - protein, 242 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 3 - protein, 45 aa, synthetic construct
TTTPAPRPPTPAPTIASQPLSLRPEACRPAAGGAVHTRGLDFACD

SEQ ID NO: 4 - protein, 25 aa, synthetic construct
IYIWAPLAGTCGVLLLSLVITLYCR

SEQ ID NO: 5 - protein, 46 aa, synthetic construct
RLKIQVRKAAIASREKADAVYTGLNTRSQETYETLKHEKPPQGSGS

SEQ ID NO: 6 - protein, 239 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSKLSKDPNEKRDHMVLLEFVTAAGITLGMDELYK

SEQ ID NO: 7 - protein, 20 aa, synthetic construct
YPYDVPDYAYPYDVPDYAWP

SEQ ID NO: 8 - protein, 251 aa, synthetic construct
HPAFLLIPQVQLVESGPGVVQPGRSLRISCAVSGFSVTNYGVHWVRQPPGKGLEWLGVIWAGGITNYNSAFMSRLTISKDNSKNTVYLQMNSLRAEDTAMYYCASRGGHYGYALDYWGQGTLVTVSSGSTSGSGKPGSSEGSTKGEIVMTQTPATLSVSAGERVTITCKASQSVSNDVTWYQQKPGQAPRLLIYSASNRYSGVPARFSGSGYGTEFTFTISSVQSEDFAVYFCQQDYSSFGQGTKLEIKTS

SEQ ID NO: 9 - protein, 243 aa, synthetic construct
QVKLQQSGAELVKPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTTVTVSSDGGGSGGGGSGGGGSDIELTQSPTTLSVTPGDRVSLSCRASQSISDYLHWYQQKSHESPRLLIKYASQSISGIPSRFSGSGSGSDFTLSINSVEPEDVGVYYCQNGHSFPLTFGAGTKLELKQTS

SEQ ID NO: 10 - protein, 66 aa, synthetic construct
IEVMYPPPYLDNEKSNGTIIHVKGKHLCPSPLFPGPSKPFWVLVVVGGVLACYSLLVTVAFIIFWV

SEQ ID NO: 11 - protein, 21 aa, synthetic construct
DGVYTGLSTRNQETYETLKHE

SEQ ID NO: 12 - protein, 42 aa, synthetic construct
RLKIQVRKAAIASREKADAVYTGLNTRSQETYETLKHEKPPQ

SEQ ID NO: 13 - protein, 42 aa, synthetic construct
RLKIQVRKAAITSYEKSDGVYTGLSTRNQETYETLKHEKPPQ

SEQ ID NO: 14 - protein, 431 aa, Mus musculus
KYLQKPMYEVQWKVVEEINGNNYVYIDPTQLPYDHKWEFPRNRLSFGKTLGAGAFGKVVEATAYGLIKSDAAMTVAVKMLKPSAHLTEREALMSELKVLSYLGNHMNIVNLLGACTVGGPTLVITEYCCYGDLLNFLRRKRDSFIFSKQEEQAEAALYKNLLHSTEPSCDSSNEYMDMKPGVSYVVPTKTDKRRSARIDSYIERDVTPAIMEDDELALDLDDLLSFSYQVAKGMAFLASKNCIHRDLAARNILLTHGRITKICDFGLARDIRNDSNYVVKGNARLPVKWMAPESIFSCVYTFESDVWSYGIFLWELFSLGSSPYPGMPVDSKFYKMIKEGFRMVSPEHAPAEMYDVMKTCWDADPLKRPTFKQVVQLIEKQISDSTKHIYSNLANCNPNPENPVVVDHSVRVNSVGSSASSTQPLLVHEDA

SEQ ID NO: 15 - protein, 431 aa, Homo sapiens
KYLQKPMYEVQWKVVEEINGNNYVYIDPTQLPYDHKWEFPRNRLSFGKTLGAGAFGKVVEATAYGLIKSDAAMTVAVKMLKPSAHLTEREALMSELKVLSYLGNHMNIVNLLGACTIGGPTLVITEYCCYGDLLNFLRRKRDSFICSKQEDHAEAALYKNLLHSKESSCSDSTNEYMDMKPGVSYVVPTKADKRRSVRIGSYIERDVTPAIMEDDELALDLEDLLSFSYQVAKGMAFLASKNCIHRDLAARNILLTHGRITKICDFGLARDIKNDSNYVVKGNARLPVKWMAPESIFNCVYTFESDVWSYGIFLWELFSLGSSPYPGMPVDSKFYKMIKEGFRMLSPEHAPAEMYDIMKTCWDADPLKRPTFKQIVQLIEKQISESTNHIYSNLANCSPNRQKPVVDHSVRINSVGSTASSSQPLLVHDDV

SEQ ID NO: 16 - protein, 176 aa, Mus musculus
CHHFHGLWYLRMMWAWLQAKRKPKKAPCRDVCYDAFVSYSEQDSHWVENLMVQQLENSDPPFKLCLHKRDFVPGKWIIDNIIDSIEKSHKTVFVLSENFVRSEWCKYELDFSHFRLFDENNDAAILVLLEPIERKAIPQRFCKLRKIMNTKTYLEWPLDEGQQEVFWVNLRTAIKS

SEQ ID NO: 17 - protein, 175 aa, Homo sapiens
HRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

SEQ ID NO: 18 - protein, 176 aa, Mus musculus
AGCKKYSRGESIYDAFVIYSSQNEDWVRNELVKNLEEGVPRFHLCLHYRDFIPGVAIAANIIQEGFHKSRKVIVVVSRHFIQSRWCIFEYEIAQTWQFLSSRSGIIFIVLEKVEKSLLRQQVELYRLLSRNTYLEWEDNPLGRHIFWRRLKNALLDGKASNPEQTAEEEQETATWT

SEQ ID NO: 19 - protein, 187 aa, Homo sapiens
KFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 20 - protein, 190 aa, Mus musculus
CLYFDLPWYVRMLCQWTQTRHRARHIPLEELQRNLQFHAFVSYSEHDSAWVKNELLPNLEKDDIRVCLHERNFVPGKSIVENIINFIEKSYKAIFVLSPHFIQSEWCHYELYFAHHNLFHEGSDNLILILLEPILQNNIPSRYHKLRALMAQRTYLEWPTEKGKRGLFWANLRASFIMKLALVNEDDVKT

SEQ ID NO: 21 - protein, 189 aa, Homo sapiens
YLDLPWYLRMVCQWTQTRRRARNIPLEELQRNLQFHAFISYSEHDSAWVKSELVPYLEKEDIQICLHERNFVPGKSIVENIINCIEKSYKSIFVLSPNFVQSEWCHYELYFAHHNLFHEGSNNLILILLEPIPQNSIPNKYHKLKALMTQRTYLQWPKEKSKRGLFWANIRAAFNMKLTLVTENNDVKS

SEQ ID NO: 22 - protein, 618 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRLKIQVRKAAIASREKADAVYTGLNTRSQETYETLKHEKPPQGSGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSKLSKDPNEKRDHMVLLEFVTAAGITLGMDELYK

SEQ ID NO: 23 - protein, 647 aa, synthetic construct
MALPVTALLLPLALLLHAARPYPYDVPDYAYPYDVPDYAWPHPAFLLIPQVQLVESGPGVVQPGRSLRISCAVSGFSVTNYGVHWVRQPPGKGLEWLGVIWAGGITNYNSAFMSRLTISKDNSKNTVYLQMNSLRAEDTAMYYCASRGGHYGYALDYWGQGTLVTVSSGSTSGSGKPGSSEGSTKGEIVMTQTPATLSVSAGERVTITCKASQSVSNDVTWYQQKPGQAPRLLIYSASNRYSGVPARFSGSGYGTEFTFTISSVQSEDFAVYFCQQDYSSFGQGTKLEIKTSTTTPAPRPPTPAPTIASQPLSLRPEACRPAAGGAVHTRGLDFACDIYIWAPLAGTCGVLLLSLVITLYCRRLKIQVRKAAIASREKADAVYTGLNTRSQETYETLKHEKPPQGSGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSKLSKDPNEKRDHMVLLEFVTAAGITLGMDELYK

SEQ ID NO: 24 - protein, 633 aa, synthetic construct
MALPVTALLLPLALLLHAARPYPYDVPDYAYPYDVPDYAQVKLQQSGAELVKPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTTVTVSSDGGGSGGGGSGGGGSDIELTQSPTTLSVTPGDRVSLSCRASQSISDYLHWYQQKSHESPRLLIKYASQSISGIPSRFSGSGSGSDFTLSINSVEPEDVGVYYCQNGHSFPLTFGAGTKLELKQTSIEVMYPPPYLDNEKSNGTIIHVKGKHLCPSPLFPGPSKPFWVLVVVGGVLACYSLLVTVAFIIFWVRLKIQVRKAAIASREKADAVYTGLNTRSQETYETLKHEKPPQGSGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSKLSKDPNEKRDHMVLLEFVTAAGITLGMDELYK

SEQ ID NO: 25 - protein, 7 aa, synthetic construct
GQSSRSS

SEQ ID NO: 26 - protein, 5 aa, synthetic construct
GGGGS

SEQ ID NO: 27 - protein, 4 aa, synthetic construct
GSGS

SEQ ID NO: 28 - protein, 4 aa, synthetic construct
GGGS

SEQ ID NO: 29 - protein, 10 aa, synthetic construct
GGGGSGGGGS

SEQ ID NO: 30 - protein, 20 aa, synthetic construct
GGGGSGGGGSGGGGSGGGGS

SEQ ID NO: 31 - protein, 8 aa, synthetic construct
GGGSGGGS

SEQ ID NO: 32 - protein, 12 aa, synthetic construct
GGGSGGGSGGGS

SEQ ID NO: 33 - protein, 21 aa, synthetic construct
METDTLLLWVLLLWVPGSTGD

SEQ ID NO: 34 - protein, 280 aa, synthetic construct
KYLLPTAAAGLLLLAAQPAMAQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGSGS

SEQ ID NO: 35 - protein, 668 aa, synthetic construct
METDTLLLWVLLLWVPGSTGDKYLLPTAAAGLLLLAAQPAMAQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGSGSEQKLISEEDLTSTTTPAPRPPTPAPTIASQPLSLRPEACRPAAGGAVHTRGLDFACDIYIWAPLAGTCGVLLLSLVITLYCRRLKIQVRKAAIASREKADAVYTGLNTRSQETYETLKHEKPPQGSGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSKLSKDPNEKRDHMVLLEFVTAAGITLGMDELYK

SEQ ID NO: 36 - protein, 12 aa, synthetic construct
EQKLISEEDLTS